(19) United States
(12) Patent Application Publication
(10) Pub. No.: US 2022/0391735 A1
(43) Pub. Date: Dec. 8, 2022

(54) USING RESILIENT SYSTEMS INFERENCE FOR ESTIMATING HOSPITAL ACQUIRED INFECTION PREVENTION INFRASTRUCTURE PERFORMANCE

(71) Applicant: The Research Foundation for the State University of New York, Binghamton, NY (US)

(72) Inventor: Lisa Sundahl Platt, Gainesville, FL (US)

(21) Appl. No.: 17/884,768

(22) Filed: Aug. 10, 2022

Related U.S. Application Data
(63) Continuation of application No. PCT/US2021/017507, filed on Feb. 10, 2021.
(60) Provisional application No. 62/972,480, filed on Feb. 10, 2020.

(51) Int. Cl. G06N 5/04; G16H 40/20

(52) U.S. Cl. CPC G06N 5/048; G16H 40/20; A61B 5/7275

(57) ABSTRACT

The present disclosure presents systems and methods for assessing hospital acquired infection reduction strategies. One such method comprises analyzing, by a computing device, a risk of hospital acquired infections, using supervised learning to generate fuzzy set membership rules; assessing resilience based on observed hospital acquired infection risk moderation performance level across a continuum of fuzzy membership sets; and inferring, by the computing device, a performance of a hospital in hospital acquired infection risk factor prevention employing the fuzzy membership set rules. Other systems and methods are also provided.

### CROSS REFERENCE TO RELATED APPLICATIONS

The present application claims priority to International Application No. PCT/US2021/017507, filed Feb. 10, 2021, which claims benefit of U.S. Provisional Patent Application No. 62/972,480, filed Feb. 10, 2020, each of which is expressly incorporated herein by reference in its entirety.

### BACKGROUND OF THE INVENTION

The disclosure of each publication and patent listed or referenced herein are hereby incorporated by reference in their entirety in this application. Such references are provided for their disclosure of technologies as may be required to enable practice of the present invention, to provide written description for claim language, to make clear applicant's possession of the invention with respect to the various aggregates, combinations, permutations, and subcombinations of the respective disclosures or portions thereof (within a particular reference or across multiple references) in conjunction with the combinations, permutations, and subcombinations of various disclosure provided herein.

The operational safety inference process described herein is meant as a framework for forecasting and considering the performance of certain types of hospital acquired infection (HAI) prevention strategies upstream of implementation. Its intended use is for estimating and evaluating the infectivity resilience potential of specific healthcare safety infrastructure. This approach is valuable for health systems serving populations and regional communities vulnerable to the effects of healthcare and community-onset infections caused by virulent pathogens such as Clostridioides Difficile and Methicillin-resistant Staphylococcus aureus.

Many HAI and their associated additional treatment expenses are imminently preventable with reliable and thoughtfully applied safety precautions (AHRQ, 2019). However, proactive HAI control risk analysis and effective infection prevention is an undertaking that is compounded in complexity by multivariate, evolving, and contextually specific circumstances. Factors that contribute to the cause of antimicrobial-resistant infection can also, at times, be unique to the type of environmental settings in which they are being considered. MRSA and C. diff. are both pathogens that frequently cause nosocomial conditions in acute care environments (Sydnor & Perl, 2011). These specific pathogens have also demonstrated the ability to reside in healthcare environments for extended lengths of time (Claro et al., 2014; Kramer et al., 2006) and are transmitted easily from an environmental surface to caregivers and thus vulnerable patients (Weber et al. 2010). Prolonged and direct exposure to these pathogens contributes to an increase in infection contraction by immunocompromised or susceptible patients (Suleyman, et al., 2018; Cassone et al., 2017; Yakob et al., 2013). For example, research suggests that length of stay, specifically in an ICU environment, has found to be a statistically significant risk factor for increasing the potential of a patient contracting a nosocomial infection (Dasgupta et at 2015).

Currently, multiple challenges exist in successfully being able to predict the probability of the occurrence of HAI as wet as nosocomial infection prevention strategy effectiveness in healthcare settings (Yanke et al., 2015). Developing retable infection risk prevention strategies that comprehensively address the operational environment of care (FOC) aspects of spreading or curtailing infection-causing pathogens in healthcare settings is a complex effort. Human behavior-based infection control interventions such as clinical staff hand hygiene campaigns indicate that compliance rates in acute care settings have plateaued at about 50% despite extensive education and adoption campaigns (McGuckin & Govednik, 2015). Environmental cleaning practices that support infection control through surface-dwelling pathogen removal in healthcare settings are also often sub-optimal (Rutala & Weber, 2013). Even with the amplified observance to the enhanced environment of care cleaning procedures, current research suggests that more than 20% of the high contact surfaces directly adjacent to patient care are not being cleaned at baseline intervals even with enhanced hospital hygiene protocols in place (Carling et al., 2008). Furthermore, in acute care settings; there can be confusion among Environmental Services staff and nursing personnel; regarding who is responsible for cleaning various surfaces and equipment in patient treatment areas (Boyce, 2016).

Deficiencies in frontline staff essential to FOG safety also appear to exacerbate the growth of HAI in healthcare settings. For example, shortages due to turn-over or worker attrition in Environmental Services (EVS) personnel of up to 50% have been reported in some healthcare facilities, and more than 50% of U.S.-based hospitals indicate significant shortages in EVS staff (Boyce, 2016). Hospital-based staffing deficiencies for nursing have also been identified as a substantial factor in hospitals' inability to deal with threats of infection spread (Stone, 2004). Additionally, increases in nursing workload have indicated a positive correlation with increased HAI, especially in critically ill patients (Hugonnet et al., 2007).

Infection Prevention within healthcare settings is a multi-faceted issue that is impacted by human factors, patient acuity, and engineered systems (See et al. 2017; Carayon & Wood, 2010). However, some evidence also suggests that the prevalence of HAI in U.S. based health facilities may also be related to factors external to the health system such as unique regional characteristics, community infrastructure, and demographic factors (See et al. 2017; McDonald et al., 2006). Additionally, surveillance of HAI causing pathogen infection incident rates has established that there appear to be relationships between the occurrence of certain types of HAI and localized factors such as exposure to agricultural waste streams in rural areas (Brown & Wilson, 2018; Freeman et al., 2010). The propensity for environmental crowding in urban areas and a deficit of healthcare access in Medically Underserved Areas (MUA) also has been cited as contributing to infection virulence (See et al. 2017).

The multivariate risk factors for HAI trajectories and impact indicate that there is a need for more systems-based analysis to determine how pathogens spread within complex care delivery settings and the local environments to which they attend. The frequency and evolving effects of both hospital and community-onset infections due to strains of bacteria with microbial antibiotic resistance require urgency in researching effective methods for improving the reliability of human health safety. Therefore, it is necessary to look more closely at the current state of infection prevention safety frameworks that characterize both the HAI risk and dangerous microbial reduction characteristics of healthcare systems and the multiple regions and populations they serve. In other words, the effectiveness of health systems' infrastructure resilience is important to moderating regional hospital-onset infectivity hazards.

Antimicrobial resistance has been attributed to operational practices such as poor antibiotic stewardship in medical prescription (CDC, 2013) and commercial farming practices (Brown & Wilson, 2018). Its persistence and growth patterns have also been linked to warmer and warming climates (MacFadden et al., 2018) and the decreased economic viability of pharmaceutical companies introducing novel antibiotic therapies (Ventola, 2015). Growing antibiotic resistance could also exacerbate the impact of diseases that have the potential to cause a pandemic. Indeed, death and illness from pandemics have already recently resulted from secondary bacterial infections from antimicrobial-resistant pathogens like MRSA, which can cause pneumonia and sepsis (MacIntyre & Bui, 2017). Therefore, bacterial antibiotic resistance likely exacerbates overall population mortality and morbidity effects from a pandemic occurrence.

The increasing prevalence of specific HAI-causing bacteria such as MRSA and C. diff. is cause for legitimate concern as both pathogens have demonstrated sustained and escalating antimicrobial resistance (Martens & Demain, 2017). Furthermore, the danger antimicrobial resistance poses to population health is compounded by the rise of community-based infections (Immergluck et al., 2019; Castro & Munoz-Price, 2019), and an aging U.S. population becoming more susceptible to HAI due to increased access of inpatient services and old age associated physiological vulnerability (Sousa et al., 2017; Solis-Hernandez et al., 2015; Avci et al., 2011). Presently C. diff. has become a severe infection control challenge for healthcare facilities due to its virulence, staying power (Clara, et al., 2014; Kramer, et al., 2006), transference efficiency in the healthcare environment (Weber, et al. 2010), and real potential for fatality in symptomatic carriers (DiDiodato & Fruchter, 2019).

Rural communities struggle with shortages of housekeeping staff due to regional population decline and aging workforces (Jaffe, 2015). The potential for patients' contracting CDI has been directly linked with the level of EGC cleanliness (Weber et al., 2013; Carling et al., 2008) as well as both static and cross-contamination (Weber et al., 2010). In one in situ Intensive Care Unit (ICU) based case study, patients admitted to an inpatient room that was previously occupied by a patient with manifesting C. diff. had a measurably higher risk for contracting CDI (Shaughnessy et al., 2011). Additionally, surfaces within the patient treatment room, portable medical equipment, blood pressure cuffs, and other fomites often become contaminated after contact with infective patients or through contact with contaminated surfaces and can be difficult to sanitize as frequently as required (Donskey, 2013).

Some other risk factors for contracting CD in an inpatient or long-term care setting include a person's physiologic vulnerability due to advanced age (Depestel & Aronoff, 2013). Gastrointestinal surgery, a long duration of stay in health care settings, and a severe underlying disease or comorbidity also contribute to higher rates and more severe effects of CDI (Schafer et al, 2004). Even the frequency of emergency department visits by patient populations has been associated with an increased rate of community-based CDI (Weng et al., 2019). These factors make CDI prevention, especially in environments which contribute to pathogen exposure, healthcare access challenged regions, and high-risk patient populations an imperative.

Patient infections caused by MRSA are the second most frequent HAI in U.S. acute care facilities (CDC, 2014). MRSA bacteria can also survive for up to four (4) months on environmental surfaces (Dancer et al., 2014; Petti et al., 2012). For example, research of MRSA counts on contact surfaces in constant proximity to vulnerable patients revealed that the bioburden of items like overbed tables and bed rails can be quite high (e.g., 30.6(0-255) colony-forming units (CFU)/100 cm2, for the overbed tables and 159.5 (0-1620) CFU/100 cm², for the bedside rails) (Kurashige et al., 2016).

In the opinion of some system resilience experts, rather than mapping discrete outcomes of emergent quality outcomes, resilience assessment requires a process of linking system adaptive capacities (Norris et al., 2007). This approach to the inference of system resilience allows for the operationalization of different adaptive responses at small or large scale and at infra or intergroup level depending upon the level of analysis. Perhaps what is most important about this approach is that it does not seek to equate resilience with outcomes, but rather with the process linking capabilities (adaptive capacities) to outcomes (successful response) (Norris et al., 2007). This method of predictive mapping to assess complex system resilience performance capabilities seems relevant to approximating the general performance of infection prevention in health systems.

A framework that maps resilience capabilities suggests that system design and behavioral qualities focused on safety preservation, and a hierarchy of technical performance measures categorize a system's performance risk moderation efficacy. These include the previously cited system performance criteria of robustness, recovery, extensibility, and sustained adaptability (Seager et al., 2017; Woods, 2015). Because these criteria indicate a continuum of system response trait qualities, it is logical to expand the notion of “Resilience Repertoire” in the Resilience Markers Model with this taxonomy. FIG. 3 illustrates the components of the hybridized Resilience Inference Model using the combined values of the Resilience Markers (Furniss et al., 2011), Military Installation Resilience Assessment (Sikula et al., 2015), and Four Concepts of Resilience Response Behaviors (Seager et al., 2017; Woods, 2015) frameworks, FIG. 3 shows Integration of “Resilience Markers”; Furniss et al., 2011), “Resilience Assessment” (Sikula et al., 2015) Four Concepts of Resilience Response Behaviors (Seager et al., 2017; Woods, 2015) Frameworks into Resilience Inference Model.

Approaches to risk management strategies typically focus on identifying and reducing the likelihood of a hazard from occurring. Because of this orientation, applied risk analysis is on event avoidance rather than event response (Husseini et al., 2016). The probabilistic approach used in risk analysis is, for the most part, reliant on what can be gleaned from data on past system threats observed and have a reasonable likelihood of occurring again in the future (Boring, 2009). Risk-based approaches such as these concentrate on achieving continuity of operations or reducing potentials for identifiable and predictable hazard outcomes (Sikula et al., 2015). Given this emphasis, the primary questions asked during a risk analysis or mitigation study are typically (Husseini et al., 2016): What systems operations could go wrong?; What is the likelihood of a hazard occurrence?; and What are the potential consequences of system threats or operational accidents?

In other words, the primary objectives of Risk Event Analysis and Risk Mitigation are on problem identification and reduction. It is a recommended practice that system threat analysis is performed at the inception of a systems operational lifecycle (Dulac et al., 2005). Indeed, according to the National Institute for Occupational Safety and Health (NIOSH) “One of the best ways to prevent and control injuries, illnesses, and fatalities is to design out and minimize hazards and risks early in the design process” (ANSVASSE, 2016). This action may not be feasible for addressing the removal of certain types of environmental or regional risks that may contribute to the prevalence of certain types of HAI. However, it is appropriate in considering what types of health system infrastructure design might be most effective in moderating the impact of infectivity risk factors. Using risk analysis in “design for safety” efforts is meant to be an iterative process where known threat assessment influences the design decisions for system as wet as system design amendments as more information becomes available and as the system evolves (Dulac et al., 2005). There are subtle nuances that exist between different Risk Analysis approaches that appear to be dependent upon whether the context of use is for systemic or discrete safety deficits assessment. The general categories that describe these approaches are identified as (Alvarenga et al., 2014): accident sequential models; accident epidemiological models; and accident systemic models.

Depending on the area of focus, it is reasonable to assume that any of these three approaches could apply to the area of hospital-onset infection avoidance. At three types of Risk Analysis models is applicable: What sequence of events or circumstances increases the potential for HAI?; How do epidemiological events contribute to the virulence of HAI?; What current conditions or assembly of system components indicate an increased potential for HAI?

Risk analysis studies the multivariate interactions of the entire system for threat aversion. Hazard potential should be viewed as resulting from, accidents that occur from the stochastic action and interaction of system component behaviors. Therefore, a hazard can potentially be avoided by monitoring and reducing behavioral variability as well as anticipating and responding in advance to future threat events (Alvarenga et al., 2014). However, it is also apparent that there is some obvious difficulty in applying a Risk Analysis and Mitigation approach to necessarily mutable systems operations like those found in healthcare delivery. Health systems should continuously consider continuity of services despite the impact of both known and unknown stressors that may impact its functional capacity and capabilities. Additionally, risk identification and management tools often rely upon predictable responses from steady-state systems when exposed to specified threats with known and identifiable hazard rates and severity (Sikula et al., 2015). The difficulty of applying this approach as the sole means of addressing antimicrobial infection prevention in healthcare delivery is dire due to the evolving and escalating nature of this issue (MacIntyre & Bui, 2017). The shortcomings of using Risk Analysis and Mitigation as a sole means of hazard response is that this combined approach may overlook low likelihood events that are still possible, some of which could have a high consequence despite their rarity (Boring, 2009).

Resilience systems engineering offers the potential to compensate for the fact that variability in complex systems is an inevitability. The emphasis of resilience assessment of system design focuses on seeking actions that can adapt to nonoptimal systems behaviors and hazardous circumstances outside system purview of control. This concern is especially pertinent in situations where a system's “defense-in-depth” is challenged. This phenomenon is characterized by the necessity of several technical, social, procedural, and behavioral layers jointly operating to maintain safe performance (Furniss et al., 2011): Such is the case in analyzing the efficacy of health systems infrastructure to prevent Hospital Acquired Infection.

A Risk or “Safety-I Hazard” Analysis is the practice of identification of root causes to determine risk likelihood (Hollnagel et al. 2013). The philosophical driver of a Safety-I approach to risk avoidance is on focusing on “what went wrong.” Its resolution is in designing procedures and safeguards to prevent or mitigate the effects of future adverse impacts from the same or similar type of occurrence. Resilience Assessment or a “Safety-II” approach is defined by the potential for human or engineered systems to succeed under varying conditions. The purpose of Safety-II is to optimize the potential for acceptable outcomes in everyday operational activities to be as high as possible (Hollnagel et al. 2013). The philosophy behind Safety-II is to leverage “what goes right” in a system and to build responsive and adaptable system infrastructure to maintain and support those abilities. Resilience assessment of system capabilities offers the possibility of augmenting traditional risk-based approaches through the incorporation of system capability evaluation to increase the potential for improving its ability to analyze and manage both known and unknown risks (Sikula et al., 2015).

System Resilience Assessment prioritizes long-term “chronic” condition management rather than “acute” issue resolution (Hollnagel et al., 2006). Hazards are viewed as context-specific outcomes resulting from interactions among human and engineered components, frequently in a system's parameter boundaries or within a system's overlapping control areas, that violate operational constraints (Dulac et al. 2005).

In describing the resilience of complex ecologies, with multiple elements impacting system states, Dr. C. S. Holling defined resilience as an ability of a given environment to return to a balanced state after a temporary disturbance (Holling, 1973). The more efficient the return to equilibrium and the less oscillation in response to a disturbance, the more resilient the system should be considered (Rolling, 1973). However, Holling also extended the definition of resilience concerning open multivariate systems to refer to the ability of a system to absorb change and disturbance and still maintain the same relationships between populations or state variables (Holling, 1973). This updated explanation of resilience alludes to a panarchy of systems behaviors that are influenced by cross-scale linkages whereby processes at one scale affect others along the continuum of interrelated elements to change the overall dynamics of the system (Allen et al. 2014). Understanding this phenomenon offers an enhanced capacity for a system to learn and evolve tom unexpected and potentially adverse events rather than merely responding to them by springing back to their original state after the disruption has passed. This progression of the meaning of resilience also illustrates that this aspect of a system's behavior is emergent. The evolution of the meaning of resilience is important to consider when seeking to apply this construct to system performance measurement in mutable circumstances. HAI incidence rates are caused by both known and unknown factors inside and outside the scope of control of health systems. Pathogen antimicrobial resistance capabilities are continually evolving at exponential rates, and the specific implications and occurrence of increased infectivity in healthcare settings cannot be precisely predicted. These qualities illustrate why infection prevention is itself an emergent quality which supports why response resilience inference as a methodology for analyzing and measuring the performance capability of health system infrastructure is appropriate. Resilience is traditionally understood as an ability or process rather than a specific outcome. Resilience is better conceptualized as a system's responsiveness and adaptability to different conditions rather than maintaining behavioral stability regardless of circumstance (Norris et al., 2007). Conceptually the approach for incorporating resilience is to understand better how system support mechanisms can effectively and reliably respond, adapt, and operate under multiple ranges and sources of variation (Hollnagel et al. 2006).

Response resilience defines the way overlapping elements that comprise social, technical, and structural/mechanical systems that instantaneously react under expected and unexpected events effectively and ideally evolve to an improved state of function (Hollnagel, 2013). Resilience Engineering describes the application of proactive, adaptive strategies and resources in the development of a socio-technical system's infrastructure to reliably and successfully respond to anticipated and unanticipated system disturbances (Sikula et al. 2015). Resilience manifests in sociotechnical systems due to the successful interaction between interrelated elements that are kept in dynamic equilibrium through various component feedback loops and information control (Dulac et al. 2005). However, most systems' abilities for resilient, adaptive capacity have boundary conditions that can exceed their designed scope of control. Exogenous and at times, endogenous variables that are either outside or just within the perimeter of a system's basis of design can create system disturbances that impact operations (Hollnagel et al., 2006). The phenomenon of variables internal to the system causing risk is especially pertinent when discussing the hazard of HAI.

Although strictly community-based infections are the catalyst for a small percentage of infection-related hospital-acquired conditions, many of these incidents are caused by circumstances internal to the hospital (CDC, 2018). In other words, HAI is often caused by endogenous within design basis factors integral to the care delivery system itself. This scenario could imply that improving the resilience of health systems infrastructure may meaningfully contribute to HAI reduction because currently, many of these infections are happening within the confines of the healthcare facility itself.

Some supervised learning analysis methods, when applied to readily available open-source data, may be instrumental in elucidating relationships that exist between factors of geographic area, rural or urban population center designation, medically underserved health service areas or populations, and specific nosocomial infection prevalence rates (See, et al., 2017; David & Daum, 2010). Supervised Machine Learning techniques could potentially assist in a better understanding of what external regional demographic factors may co-occur with HAI incidence (Ehrentraut et al., 2012). Specific data analysis approaches may serve as vehicles for revealing ways to augment health systems decision making in determining what operational and structural factors may be most effective in moderating HAI spread in acute care environments (Forrester et al., 2006). Associations exist between regional demographic factors within a hospital's patient catchment area that could aid in informing strategies related to HAI prevention.

Some of the benefits of using Supervised Learning for risk analysis include (Obenshain, 2004): the ability to examine multiple areas simultaneously; a decreased potential for human error in result interpretation; and using relatively accessible data repositories.

current research using data surveillance techniques have demonstrated an apparent relationship between the incidence of HAI and demographic factors such as the propensity for environmental crowding in urban areas and the deficit of healthcare access in Medically Underserved Areas (MUA) (See et al. 2017). Studies have also suggested that using this approach for risk event, and risk mitigation analysis for improving EOC safety can assist in reliably detecting patient colonization rates as well as assessing the efficacy of strategies meant to stem pathogen transmission within acute healthcare settings (Ehrentraut et al. 2012; Forrester et al. 2007). Applying data analysis methods to regional and specific HAI prevalence data (e.g., open-source data) offers a viable pathway for more U.S.-based health systems to gain increased clarity around external sources that could incite incidences of HAI within healthcare settings.

There are opportunities to mine and parse readily available and publicly reported demographic data related to geographic characteristics and medically underserved patient populations and compare these to regional de-identified patient HAI incidence rates to reveal the existence of apparent linkages that may exist between these factors. Applying these methods to regional and specific HAI prevalence data may offer a pathway for more U.S.-based health systems and population health improvement stakeholders and organizations to gain increased clarity around external sources that could incite infectivity risks within their specific service area communities. Additionally, these tools could also serve as a decision support mechanism for healthcare facilities to develop internal strategies and environment of care infrastructure effective in reducing nosocomial infection transmission within acute healthcare settings.

Data analysis methods have also been used as an aid in testing health system infection control and resilience capabilities. Specific research on this topic suggests that employing supervised learning data analysis techniques for enhancing infection control capabilities offers systems far more sensitivity in pinpointing infection cause and occurrence than using traditional infection control surveillance methods (Obenshain, 2000. Additionally, the mining and analysis of longitudinal data on system hazard response and disaster readiness can also offer insight into the codification of resilience traits and thus, performance baseline metrics (Norris et al., 2007).

Ordinary Least Squares (OLS) regression, which is a type of supervised learning method in data analysis, has also proven a useful tool in guiding the development of system performance inference rules. The statistical method of orthogonal transformation was initially designed for linear optimization in data analysis. However, it can also serve as a vehicle for component feature selection and building rules from data and selecting a limited subset of rules for inference models (Destercke et al. 2007) There is a precedence of combining supervised learning data analysis techniques such as OLS with Fuzzy Inference models (Khaderni et al., 2017; Ubale & Sananse, 2016; Destercke et al. 2007). Using a technique such OLS regression in data analysis allows a for fuzzy rule selection to be based on independent variable contributions to dependent variable inertia or variance and thus offers a good summary of the system to be modeled (Destercke et al, 2007).

Lofti Zadeh developed fuzzy logic as a response to dealing with the inherent indistinctness of certain variable boundaries (Klir & Yuan, 1995). Fuzzy logic is based on the concept of traditional set theory (Mendel, 1995). However, unlike classical set theory which is based on bivalent logic where an element is either a member of a set (e.g., 1) or not (e.g., 0) fuzzy logic allows a number or object to have partial membership (e.g., 0-1) in more than one set (Konstandinidou et al., 2006). Fuzzy Logic's approach specializes in dealing with uncertainty and imprecision of reasoning processes and situations as it allows the modeling of potentially valuable heuristic knowledge which cannot be described as effectively by classical mathematical equations (Grecco et al., 2013). One of the most beneficial aspects of human thought incorporation in the analysis is the ability to summarize information into categories of fuzzy sets which bear an approximate relation to the source of the data (Dubois, 1980). These categorical sets are typically comprised of summative descriptions of complex circumstances which can vary depending on contextual comparisons, and themselves have imprecise boundaries. Fuzzy Logic's methodology, metric delineation, and application mimic that of natural human language, which allows for the computation of more nuanced linguistic information (Zadeh, 1996). See also, en.wikipedia.org/wiki/Fuzzy_logic.

Reasoning processes and situations that fuzzy inference is applied to in healthcare settings allows for the accrual of potentially valuable heuristic knowledge from various process Subject Matter Experts (SME) (Leite et al., 2011). Fuzzy Inference offers a unique ability to provide an appropriate logical-mathematical framework to handle problems with vague and fluctuating circumstances, such as infection prevention resilience. Implementation of Fuzzy Logic benefits Resilience Inference and control since it is guided by naturalistic human thought and decision making. In practical yet complex scenarios where the choice of actions may be simultaneously dependent on utility values, expected consequences of operations, and states of nature, both information and response outcomes may be fuzzy (Dubois and Prade, 1980). Fuzzy sets formation could also include human response limitations driven by informational, cognitive, and temporal constraints (Elsawah et al., 2015; Smith and Eloff, 2000). This consideration is especially relevant in safety-critical and high-stress operational settings like healthcare, where a human actor's decisions and actions figure prominently in the causality of system interactions and outcomes (Rothblum, 2000).

Fuzzy Inference has had nascent success in facilitating the accrual of specialist knowledge in high-risk critical care settings to develop procedures for improving patient safety outcomes (Leite et al., 2011). The ability to facilitate interdisciplinary collaboration especially among clinical care providers is cited as an essential consideration in developing mechanisms and methods to support evolving direct patient care needs (Fronczek & Rouhana, 2017; King 2007). This human-centered approach to pursuing systems design based on practical and communicated stakeholder need, along with its iterative data-informed framework ensures that low-level design responses achieve high-level design requirements. The benefit of using a fuzzy approach to decision, support is that it provides the opportunity to accommodate uncertainty in the evaluation of essential system attributes and brings together different measurement scales to offer a combined outcome (Muter, 2012). This approach could facilitate an increased potential for system resilience, reliability, and ideally stakeholder satisfaction with system response outcomes.

The application of fuzzy inference to improve system safety within environments of care delivery has had prior use in preventative healthcare safety improvement efforts. (Leite et al., 2011). Fuzzy logic addresses meaningful qualitative data about system performance effectively since it resembles the way humans make inferences and decisions (Zadeh, 1996). In planning health systems infrastructure that may help to reduce the incidence of HAI multi-stakeholder model development process comprehension is critical. This position is relevant because; any system analysis process meant for application to actual work process or structural design improvement must be able to be easily comprehended by the various group of participants essential to that project goal's successful realization (Latham & Locke, 2007). Human trust is directly related to changes in the performance of a system and trust can be mathematically and accurately predicted by understanding system errors (Khasawneh et al.; 2003). Because fuzzy inference and logic models reflect models of human thought and process descriptors their classifications of system risk potentials and error mitigation capabilities could offer system stakeholders an enhanced understanding of general and complex system performance.

Infection prevention is a pressing, mutable, and escalating problem in healthcare that presents an intuitive opportunity for proactive health systems safety improvement. One of the most challenging things about designing processes and environments meant to drive performance outcomes in complex and dynamic systems is understanding how the different components of the system interact with one another to achieve specific goals. This occasion exemplifies the opportunity applied fuzzy logic offers for preemptively identifying risk prevention opportunities and developing health infrastructure and resources capabilities that are capable of resilient performance outcomes.

### SUMMARY OF THE INVENTION

The Resilience Inference System for Performance Safety (RISPS) Process Model is an algorithmic framework which enables healthcare organizations to forecast potential outcomes of operationally based infection control interventions in acute care settings. The RISPS Process Model projects operational performance safety outcomes based on the intersection of data driven possibilistic metrics of infection prevention risk and autonomous system adaptive response (i.e., resilience). Its purpose is to enable healthcare quality and safety teams to gain meaningful and accurate insight into the possible performance safety level of environment of care infection prevention strategies through the application of a series of System Science derived mathematical models.

The ability to predict and prevent the occurrence of HAI continues to be an evolving challenge both in the U.S. and around the world. Care delivery environments meant to support curative efforts can often be a significant source of infectivity risk. This issue is often due to the inherent complexity and combined influences of healthcare settings themselves, the community of patients that they serve, and the geographical and socioeconomic region in which they must operate. The present technology validates the concept of exploring geographic and demographic data for determining Clostridioides difficile and Methicillin-resistant Staphylococcus Aureus HAI risk factors by geographic region.

Targeted HAI resilience strategies can be mined from evidence-based case study literature and incorporated into nested fuzzy technical performance membership system attributes for evaluating system performance safety. Supervised Learning techniques such as OLS offer greater specificity to the weighting of different system risk and resilience fuzzy membership levels. An approach is provided for using Resilience Inference Fuzzy Membership Categories based on Fuzzy Risk Capacity, Resilience Capability, and Performance Safety outcomes as a basis for Fuzzy Inference System decision rules. The methodology establishes a process for inputting fuzzy HAI Risk and HAI Resilience membership function parameters into a Fuzzy Inference System that could estimate specific HAI Performance Safety outcomes.

Due to the increasing complexity of systems in safety-critical organizations like acute care health systems, there is an urgent and growing need for anticipatory rather than reactive operational performance response. The RISPS model provides a process that healthcare practitioners can use in enhancing HAI reduction strategies in acute care settings. The RISPS framework serves as a decision support instrument by forecasting possible outcomes of combined HAI risk mitigation capacity and Infection Prevention resilience capability in inpatient care delivery settings. The RISPS Process Model uses a combined approach of system Risk Analysis and Resilience Assessment, to generate Performance Safety inference outcomes of domain agnostic, but context specific, environment of care settings. The mathematical models it uses for system analysis and prediction are derived from systems science and include supervised learning techniques and fuzzy logic.

The RISPS framework allows healthcare Quality, Health, Safety and Environment (QHSE) Management teams the ability to consider the performance outcomes of certain types of HAI prevention strategies through resulting predictive outcomes upstream of implementation. The RISPS framework is for estimating and evaluating the infectivity resilience potential of specific healthcare safety infrastructure. This approach is valuable for health systems serving populations and regional communities vulnerable, to the effects of healthcare and community-onset infections caused by virulent pathogens such as those with the potential to remain within environments for extended periods of time and associated adaptive systems response outcomes. More resilient healthcare infrastructure tailored to community safety and risk mitigation priorities may arise from optimal state health safety requirements, regionally specific healthcare design and building codes, and demographically specific population health improvement efforts.

RISPS enables assimilation of factors that contribute to the cause of antimicrobial-resistant infection unique to the type of environmental settings and regional and demographic context in which they are being considered. The framework is built from the integration of supervised learning techniques applied to geographic and sociological data as wet as fuzzy logic for developing a resilience inference process model that estimates infection prevention performance safety of health system infrastructure to serve as an environmental scanning method for predicting HAI risk prevention capacity.

This model facilitates the incorporation of targeted HAI resilience strategies mined from evidence-based data and incorporated into nested fuzzy technical performance membership system attributes; for evaluating system performance safety. It also integrates an approach for weighting of different system risk and resilience fuzzy membership levels. The analysis reveals an approach for using Resilience Inference Fuzzy Membership Categories based on Fuzzy Risk Capacity, Resilience Capability, and Performance Safety outcomes as a basis for Fuzzy Inference System decision rules. Finally, the RISPS Process model establishes a process for inputting fuzzy HAI Risk and HAI Resilience membership function parameters into a Fuzzy Inference System that could estimate specific HAI Performance Safety outcomes. There are three primary methods of system evaluation and predictive analysis that are guiding the operational structure of RISPS Process Model. These include; Risk Analysis: using supervised learning techniques on regional data, and Fuzzy Logic to operationalize the combined processes of: Risk Event Analysis: processes of Risk Event Identification and System Likelihood of Hazard Exposure Determination; Risk Mitigation Evaluation: Risk Parameter Assessment, Likelihood of Risk Exposure Stability, and Risk Event Reversibility; and Risk Prevention Capacity: attribution of the level of system HAI hazard prevention capacity based on Risk Event exposure and Risk Mitigation opportunity; Resilience Assessment: how to measure the Resilience Repertoire of a system which includes a system's Risk Management Inventory, Risk Avoidance Resources, and Resilience Strategy Assessment based on their observed HAI risk moderation performance level across a continuum of Fuzzy Membership sets; and Performance Inference: the process for evaluating the impact of resilient procedural inventory and system resource performance capability based on regional health system HAI risk factor prevention capacity using applied Fuzzy Inference Systems. An illustration of the Resilience inference methodology process explanation segments is delineated in FIG. 4.

An overlooked, but potentially impactful system component to consider in healthcare infection control planning potential use of existing relevant data repositories related to environmental contextual factors to guide the design for reliable infection prevention infrastructure. This ensures elements of preventive support are not only included in the ongoing planning for structures supporting care delivery but that informational feedforward and feedback mechanisms are embedded into an operational process to serve as vehicles for continuous improvement within care system lifecycles (ANSI/ASSE, 2017).

The present technology may be integrated into complexity modeling and simulation platforms such as Agent Based Modeling and Systems Dynamics. It is developed for use of quantitative regional and demographic data as well as heuristic clinical and operational subject matter expertise for forecasting operational safety performance outcomes. Systems Science derived methods are used to improve reliability of predictive outcomes. It is designed for use in environment of care master planning and discrete inpatient unit design.

Products and/or services that might benefit from RISES technology and potential end user(s) include an infection control implementation strategy forecasting platform, with the end user being Infection Control Teams; safe environment of care planning support simulation platform, with the end user being a facility operations team, AEC and healthcare design community; healthcare facility operations risk assessment and mitigation planning dashboard, with the end user being quality, health, safety and environment management teams.

Supervised Learning techniques applied to geographic and sociological data as well as Fuzzy Logic for developing a Resilience Inference process model that could estimate the infection prevention performance safety of health system infrastructure serves as a viable environmental scanning method for predicting HAI risk potential. Supervised Learning data analysis techniques have demonstrated effectiveness in the discovery of regional and population health factors associated with HAI incidence. Applied Fuzzy Logic has a unique ability to provide rational, mathematical frameworks for complex dynamic problems with vague and contextually mutable circumstances. Integrating Fuzzy Inference Systems to inform Infection Control strategies offers a unique vehicle for health systems to plan more reliable safety-critical healthcare infrastructure based on specific and system priority risk factors. Open-source publicly reported data may be used in training the system, e.g., regarding Clostridioides Difficile (C. diff.), and Methicillin-resistant Staphylococcus aureus (MRSA) observed hospital-onset infections in U.S. based acute care hospitals as two dependent variables for analyzing regional risk factors. Additionally, it uses both dependent variables as focal points for exploring the interaction effects of infection prevention resilience strategies. The analysis of regional and demographic-based Risk and system intervention Resilience factors are combined to form a fuzzy rule basis. The purpose of this rule basis is for use within a Fuzzy Inference Systems model to evaluate the safety achievement level of infection control measures based on the combined effect of acute care infectivity risk and associated resilient systems inputs.

The definition of resilience is the ability of an organization to anticipate, forecast, manage, and avoid hazards and threats to its primary performance goals (Hollnagel et al., 2006). The term health system infrastructure in the context of this document is meant to represent both human and engineered systems and resources intended to deliver or support safe patient care (IOM, 2002). Health infrastructure in the context of infection control would then be those human and engineered systems which sought to support the delivery of safe and patient-centric care as wet as anticipate, predict, manage and avoid hazards and threats related to hospital-onset infection.

Predictive data analysis frameworks have demonstrated reliability in detecting pathogen colonization rates in specific patient groups as wet as assessing the efficacy of strategies meant to stem microbial transmission within acute healthcare settings (Ehrentraut et al. 2012; Forrester et al. 2006). Traditional discrete system analysis methods that have been employed by many health systems to track the probability of HAI incidence internally are labor-intensive, expensive and therefore, often infeasible for sustained use by resource-poor health systems. Furthermore, many of these analytic approaches offer only a retrospective view of constrained historical data and cannot project potential future combined system behaviors or test alternative prevention scenarios. Two central challenges in stemming the pervasiveness of antimicrobial-resistant infections are (Shekelle et al., 2013): predicting the probability of risk in infection occurrence in prospective patient populations and care community settings; and improving the resilience of care delivery-systems infrastructure to infection prevention adaptive response.

The need to examine the multi-causal problem of HAI and community-onset infections from a variety of perspectives concurrently suggests that methods effective for complex system analysis are an important component in elucidating relationships that may exist between factors such as features endemic to certain geographic areas, unique patient population characteristics, care accessibility, and specific hospital-acquired and community-based infection prevalence rates. These system assessment methodologies could serve as vehicles for health system stakeholders gaining a clearer picture of current regional population infectivity risk factors and for evaluating what types of safe care delivery infrastructure may be needed for active infection hazard adaptive response.

Resilience Inference is used to gain a clearer understanding of what population, regional, and community characteristics may coincide with increased MRSA and C. diff. HAI rates in acute care settings. A methodology is provided for health systems and population health stakeholders to use to assess better the performance safety potential of their care delivery infrastructure to be resilient to antimicrobial infection risk.

Relationships that may exist between the regional geographic characteristics, population demographics, and health access characteristics are analyzed by U.S. state (e.g., Rural/Urban Designation Proportion, Population Density, Medically-Underserved Area and Population, etc.) and the trajectory of observations of HAI caused by specific antibiotic-resistant pathogens (e.g., MRSA and C. diff.) in specific inpatient care settings (e.g. acute care hospitals). Open-source data on healthcare catchment area designation is available for access from the Health Resources and Services Administration's (HRSA) query repository, as well as data from the United States Census Bureau on regional population demographical characteristics, and state-based statistics from The United States Interagency Council on Homelessness which may be compared to the CDC's National Healthcare Safety Network (NHSN) data on State-specific Healthcare-Associated Infections observed incidence rates in acute care hospital settings.

The present technology uses Risk Analysis techniques to assess if there are statistically meaningful co-occurring relationships of risk that exist between U.S. regional geographic and demographic factors and MRSA and CDI hospital-onset incident rates that manifest in the analysis of environmental, population demography, and health data. The present technology uses Resilience Assessment to evaluate how these risk analysis outcomes may relate to a representative health system's infection prevention infrastructure adaptive capacity for stemming the adverse effects of hospital-onset infectivity. The present technology further uses fuzzy logic methods for generating Performance Inference safety estimates for health system infrastructure based on related HAI risk and resilience inputs. This method proposes a process for ascertaining what levels of health infrastructure resilience may be most effective in moderating certain types of HAI spread in acute care environments based on relevant regional HAI type risk factors.

FIG. 1 shows Resilience Inference Map of Objectives. The analysis methodology is a nonexperimental design that investigates the effects of different contextual circumstances data on antimicrobial-resistant (AMR) HAI incident rates by U.S. region. It is considered nonexperimental because of the lack of random variable assignment, or manipulation, as wet as lack of a control group. Nonexperimental methods are often considered to be weaker in their validity for determining causation (Trochim, 2006). However, there has been prior use of this type of approach to determine human mortality and morbidity risk factors based on situational and environmental contexts (Merlo et al., 2013; Fernandez et al., 2011). Moreover, this research uses available database information relatively unexplored for AMR hospital-acquired infection prevention.

The concept of system resilience is inexorably linked to the potential and impact of risk. Risk Analysis hypotheses may be extended to include a specifically relevant resilience strategy meant to anticipate, forecast, manage, and avoid the risk of either CDI or MRSA HAI. New hypotheses in the form of a fuzzy rule basis for resilience inference and HAI performance safety may be formed and tested on an experimental basis using a Fuzzy Inference System.

There are systems science derived methods like fuzzy logic that are well suited to evaluating system performance that is vague but contextually specific. Additionally, there is data embedded in epidemiological, material science, health safety, and public health research whose outcomes can be used as quantitative benchmarks for infection reduction capabilities.

Using the above described evidence-based metrics and Fuzzy Inference approach allows the extension of Risk Analysis fuzzy rule basis hypotheses by integrating resilience strategies and using both Risk and Resilience data as inputs to make inferences on HAI specific speculative performance safety outcomes. Resilience Assessment and Performance Safety inference are directly linked to the formation of a fuzzy rule inference basis for estimating hospital-acquired infection prevention infrastructure.

A fuzzy logic assessment of the Resilience Assessment Hypothesis is:

IF HAI infection exposure Risk is High and Prevention Low, THEN infection prevention Resilience must be Strong to moderate the effects of HAI risk.

A fuzzy logic assessment of the Performance Inference Hypothesis is:

IF Risk Prevention is Low AND Resilience is Strong THEN Hospital Onset Infection Control Performance is Safe.

“Designing for Safety,” as defined by the American National Standards Institute (ANSI) and American Society of Safety Professionals (ASSP; formally ASSE), advocates that approaches using “state of the art [systems] engineering and management” be used when planning safety-critical systems (ANSI/ASSE, 2016). An overlooked, but potentially impactful system component to consider in healthcare infection control planning is how we may use existing relevant data repositories related to environmental contextual factors to guide the design for reliable infection prevention infrastructure. That is, the technology disclosed herein is directed toward determining what infection control measures may be appropriate, to what extent, and when, based on datasets which are contextually applied and may be normalized. The purpose being, to ensure elements of preventive support are not only included in the ongoing planning for structures supporting care delivery but that informational feedforward and feedback mechanisms are embedded into an operational process to serve as vehicles for continuous improvement within care system lifecycles (ANSI/ASSE, 2017). In Resilience Engineering, systems operational components are viewed as interrelated elements that are kept in equilibrium through various feedback loops and information control (Dulac et al. 2005).

Systems resilience is reliant on the strategic and practical application of both objective data and heuristic knowledge in response to circumstances that are inexact and complex. The use of metadata to inform risk and Resilience Inference to guide infection prevention planning in acute care is an underutilized but promising approach. This process may assist in improving the understating of how to predict resilient system design outcomes based on external but influential factors.

The infection prevention through resilient systems performance inference framework is meant to serve as a guide for developing support for safe care delivery and health system infrastructure planning. The components of this model include an associated rule basis that is integrated into a context-specific, but domain agnostic, systems analysis models. Given the prevalence and growth trajectories of HAI causing multidrug-resistant organisms (MDRO) (Ventola, 2015; CDC, 2013) there is an imperative for assessing how methods of analysis regarding factors outside health systems design basis may influence human health outcomes (Merlo et al., 2013; Fernandez et al., 2011; Epstein, 2002).

A nonexperimental research design is used to study the relationships between HAI and remedial efforts. The methodology examines how a series of U.S. based environmental and demographic independent variables affects the dependent variables of prior occurring CDI and MRSA HAI outcomes in U.S. acute care hospitals.

In the parlance of Systems Science and Engineering, the construct of “Resilience” describes the application of proactive, adaptive strategies and resources in the development of a socio-technical system's infrastructure to reliably and successfully respond to anticipated and unanticipated system disturbances (Sikula et al., 2015). The objective is that the property of resilience not only allows elements of a system to sustain function under expected and unexpected events efficiently but allows the system to learn from disruptive events bringing system sentience to a new state (Hollnagel, 2013). In complex circumstances meant to address acute or chronic safety issues, as well as prioritize performance goals, finite resources are apportioned to respond with proactive resilient processes rather than through reactive barriers and defenses (Hollnagel et al., 2006).

Resilience assessment in healthcare delivery is often referenced in terms of care delivery safety and performance management. High-reliability system performance applied to improve the safety and performance of infection control measures requires an acknowledgment of inherent system complexity and ambiguity. Effective infection prevention requires both highly reliable and contextually adaptive behavior (Chassin & Loeb, 2011) from both the humans delivering care and the environmental resources designed to support care delivery.

For systems to sustain performance resilience, risk and opportunity is managed preemptively, and operational adaptive responses evolved (Hollnagel, 2013). In considering the sociotechnical and environment aspects of system performance, resilience is seen not just as an assembly of individual components focused towards the goal of achieving a successful adaptive response to system disruption. Resilience in complex systems such as those functioning in healthcare and population health improvement accepts and confronts the ongoing trade-offs to maintain optimal system performance (Seager et al. 2017). The ability of successful and streamlined interaction between system elements guided by a superordinate socio-technical context is what truly makes a system resilient (Clauss-Ehlers, 2003). A superordinate framework of shared perspective resilience can also facilitate the understanding of systemic thinking across organizational stakeholder groups. This issue is especially important in uncertain performance impact categories (Matzenberger, 2013), such as infection prevention in healthcare settings.

System resilience contextual markers are an aspect of identifying the contributory characteristics of a system's capacity for resilience. “Resilience Repertoire” in an organization determines that a priority of operational needs is sustainably supported by appropriate resources, system characteristics, and functional structures (Furniss et al., 2011). This rubric facilitates a superordinate socio-technical Resilience Inference framework that links abstract theory to concrete observations and vice versa (ref. FIG. 4.) (Furniss et al., 2011).

FIG. 2 shows Resilience Markers Framework. (Adapted from source: Furniss et al., 2011). Systems resilience also suggests the execution of four actions by a system regardless of its complexity level: Sensing, Anticipating, Adapting, and Learning (SAAL) (Seager et al, 2017). An approach to using Resilience Inference for improving systems performance argues that a system must be able to respond, monitor, learn, and anticipate. To be in a perpetual state of anticipation necessitates that a system can consider itself and reflect on its response impacts in terms of its internal and external performance and outcome influences (Hollnagel, 2013).

Resilience is not only about being vigilant or robust to potential disturbance but also about readiness to recognize opportunities that these unique disturbances offbr in terms of recombination of structures and processes which could facilitate operational transformation and outcome trajectory change. The aspect of considering internal system outcomes that might be contingent upon external variable characteristics provides a point of overlap to consider in establishing the operational criteria necessary for supporting a Resilience, Repertoire.

Assessing potential system vulnerabilities and risk factors are an inevitable and continuous part of managing the safe and resilient performance of complex and dynamic systems. Organizations such as healthcare are particularly susceptible to this due to the type of services they are providing and the multivariate and mutable nature of the environments in which they are operating. A potential method for establishing a ranking for overall system resilience is through it perceived response to risk. One methodology that is especially useful in parsing both safety-related causes and outcomes and offering viable solutions for error mitigation is Risk Analysis.

Supervised Learning methods used in the analysis of available population health and demographic data could provide a useful and accessible way for health systems to conduct a risk analysis. This approach uses the values of several variables (inputs) to make predictions about another variable (target) with identified values (Obenshain, 2004). This objective is particularly relevant to the effort of analyzing available data to determine what factors external to health systems operations may be related to the trajectories of internal documented CDI and MRSA HAI incidences. This process elucidates what types of resources may offer the most significant support for moderating AMR HAI occurrence.

Heuristics based on historical precedence alone are insufficient as a singular basis for developing strategies in complex systems meant to foster adaptive capacity and response resilience (Seager et al, 2017). The question of instilling system resilience should not solely be which individual improvements that will facilitate a specific end. A better strategy for improved [resilient] performance is to consider what general areas of system leverage can be optimized in an organization (Gilbert, 2007). Holistic evaluation of wide-ranging contextual factors of a system help achieve resilience. The adoption of applied resilience as a pathway for achieving sustainable quality outcomes in engineered systems has been relatively recent in healthcare improvement initiatives (Hollnagel et al., 2006). The primary application of improving resilience in healthcare delivery has been in augmenting policies to care delivery process safety (Hollnagel et al., 2013). Typically, resilient outcomes require the strategic investment of both human and operational resources and actions (Seager et al., 2017). To stem the trajectory of AMR CDI and MRSA successfully in acute care, both structural environment designs and engineered resource planning should also be addressed.

Measurement of system resilience parameters can be somewhat more challenging than identifying risk. Adaptive capacity within a system does have limits or boundary conditions, and internal and external disturbances that impact the system operations provide information about where those boundaries lie and how the system behaves when events push it near or over those boundaries (Hollnagel et al., 2006). The core construct of resilience is concerned with understanding how effectively, efficiently, and reliably a system adapts to multiple ranges and sources of variation. (Hollnagel et al., 2006). Adopting an investigative process that evaluates risk probabilities as they relate to socio-ecological system resilience principles offers the opportunity of expanding traditional risk analysis as well as the potential avenue for improving agency abilities to assess and manage both known and unknown risks (Sikula et al., 2015).

This approach considers both the role of independent and overlapping subsystems as wet as how singular or multiple panarchies may influence system performance outcomes. A “panarchy” is a classification of interrelated and symbiotic elements that characterizes complex human, ecological, and environmental systems that are dynamically organized and structured within and across scales of space and time (Allen et al., 2014). According to the Resilience Markers Framework, both Risk Analysis and Resilience Assessment contribute to the potential of inferring the characteristics of a system's current state “Resilience Repertoire.” A resilience repertoire includes the skits, strategies, and competencies that direct a system's responses to threats and vulnerabilities which are outside design-basis (Furniss et al., 2011). If the danger supersedes system capabilities and its resilience repertoire is inadequate or brittle in its response, the system performance will deteriorate or fail. To evaluate the capacity of a system's resilience inventory, the capability traits of its combined resources must be assessed.

System resilience ability can, in part, be characterized by the technical performance capability traits of “Robustness, Recovery, Graceful Extensibility, and Sustained Adaptability” (Seager et al., 2017; Woods, 2015). “Robustness” and “Recovery” are relatively straightforward in their meaning. Robust system qualities refer to the capacity of a given system to firmly resist shocks or stressors without significant system degradation or failure. Recovery for any critical system function might be considered as a form of an adaptive response to hazards (Seager et al., 2017; Woods, 2015). The explanation of the characteristic of “Graceful Extensibility,” however, is a bit more nuanced. This attribute is essentially the degree to which a system is prescient to unknown, unanticipated circumstance. Therefore, systems containing this characteristic should seek to manage in advance the consequence of both known and unknown hazard to avoid brittle response and catastrophic failures (Seager et al., 2017; Woods, 2015). “Sustained Adaptability” is the acknowledgment and acceptance that none of the other individual criteria characterizing system resilience on its own will be successful over the long term, regardless of the frequency of past successes (Seager et al., 2017; Woods, 2015). The hierarchy of resilience repertoire performance measures of “Robustness, Recovery, Graceful Extensibility, and Sustained Adaptability” (Seager et al., 2017; Woods, 2015), could represent linguistic variables that define the fuzzy performance qualities in that their definitions are both vague and context-specific (Dubois & Prade, 1980). In other words, this continuum for measuring resilience repertoire abilities could also be extrapolated to infer infection prevention performance fitness within health systems.

Concepts such as operational “Risk” and “Resilience” are difficult to describe adequately by classical mathematical equations alone. Although strictly quantitative methods, such as surveys, mathematical modeling, and computer simulations are useful in systems' behavior analysis the complexity of real settings is difficult to be evaluated through those methods alone (Righi et al., 2015). There has been precedent research by experts in systems resilience measurement that estimates speculative performance via mathematical functions as a basis for parametric comparison of a proposed resilience metric with an integration-based parameter (Tran et al., 2017). However, fuzzy inference systems offer the ability to integrate both quantitative data and qualitative perceived levels of system capabilities through a series of rule-based functions to find the centroid of the combined performance output vector. Using Fuzzy Logic in risk analysis and resilience assessment offers the potential evaluate the levels of risk exposure within a system and rank them across a continuum of generalized fuzzy membership categories (Chen & Chen 2009). There is also a precedence of fuzzy inference systems being applied in healthcare systems to harness expert knowledge to improve patient safety (Leite et al., 2011). If used within the context of HAI prevention in healthcare settings this approach allows for the accrual of potentially valuable heuristic knowledge from various subject matter experts which is a consideration in evaluating the potential implementation outcomes of infection control resilience resources.

Lofti Zadeh introduced the idea of using Fuzzy Logic for systems control, to improve the applicability of systems control methodology and design to practical “real world” problems (Lewis, 2013). Fuzzy Inference Systems (FIS) combine variable Fuzzy Set membership functions with Fuzzy Control rules to derive crisp outputs indicative of system performance (Bai and slang, 2006). Fuzzy Inference involves the applied use of fuzzy operators, including membership functions, fuzzy logic operators, and if-then rules. This proposed method is predicated on the building of fuzzy modalities, which allows for the creation of fuzzy values from a predefined set of data. The decision rules which are derived from systems-based fuzzy membership function relationships are formulated using an IF and THEN structure, in which the IF part specifies the quantitative variable and THEN part determines the technical performance level (Anooj, 2012). For example, in considering FIS applied to the premise of infection prevention through a resilient performance framework, an example of a primary decision rule could be: IF HAI infection exposure risk is high AND prevention low, THEN infection prevention resilience must be strong.

An approach which can be used in fuzzy inference to construct Resilience Assessment decision rules is through the extrapolation of linguistic terms to categorize resilience repertoire performance indicators by systems experts. These linguistic terms can be transformed into fuzzy membership categories to represent the degree to which each resilience performance criteria is met (Grecco et al., 2013). These membership functions could then be generalized into rule structure development to drive strategies for systems adaptive capacity based on how systems experts interpret the characteristics of optimal system functioning (Nivolianitou & Konstantinidou, 2018). The benefit of this approach to Resilience Inference is the ability to leverage the intuition, knowledge, and experience of practiced operators to establish a degree of resilience performance safety achievable by system design. Such an approach could provide more nuanced information about where disturbances boundaries lie and how system parts behave when circumstances drive them beyond their design basis of control (Hollnagel et al., 2006.)

It is therefore an object to provide a method for assessing strategies, comprising: analyzing an outcome-related risk of a set of strategies, each strategy comprising strategic risk factors, using supervised learning on strategy context-appropriate data, to generate fuzzy set membership rules; assessing resilience, based on observed performance of a strategy across a continuum of fuzzy sets; and inferring a performance of each of the set of strategies using a fuzzy inference system employing the fuzzy membership set rules.

The method may further comprise adaptively modifying at least one fuzzy set membership rule in dependence on the inferred performance. The analysis of the outcome-related risk may comprise selecting risk features. The strategic risk factors may be selected according to a principal component analysis. The fuzzy set membership rules may define a risk prevention continuum. The analysis of the outcome-related risk may be location-dependent. The assessing resilience may comprise determining a risk management inventory and/or determining a risk avoidance resource. The fuzzy set membership rules may define a risk prevention continuum. The analysis of the outcome-related risk may comprise use of fuzzy cognitive mapping to increase reliability and validity of risk mitigation strategies.

It is also an object to provide a method for assessing a risk, comprising: determining a set of fuzzy inference system rules associated with resilience inference fuzzy membership categories, based on at least fuzzy risk capacity, resilience capacity, and performance; receiving information about the risk and resilience inference fuzzy membership category function parameters; and employing a fuzzy inference system dependent on the fuzzy inference system rules and the received information to predict performance outcomes.

The risk may be derived though machine learning and heuristics. The risk may be derived though machine learning and fuzzy cognitive mapping. The risk may be derived through at least machine learning to increase a reliability of determination of risk event occurrence. The risk may be derived through at least one of heuristics or fuzzy cognitive mapping increase a validity of at least one risk mitigation strategy. The method may further comprise using fuzzy cognitive mapping to assess a stability of an event associates with the risk. The method may further comprise using fuzzy cognitive mapping to assess a reversibility of an event associates with the risk. The determining the set of fuzzy inference system rules associated with the resilience inference fuzzy membership categories may comprise supervised learning on labelled data.

It is therefore an object to provide a method for assessing hospital acquired infection reduction strategies, comprising: analyzing risk of hospital acquired infections, using supervised learning on context-appropriate data, to generate fuzzy set membership rules; assessing resilience, based on observed hospital acquired infection risk moderation performance level across a continuum of fuzzy membership sets; and inferring a performance of a hospital in hospital acquired infection risk factor prevention using a fuzzy inference system employing the fuzzy membership set rules.

The risk features may be selected according to a principal component analysis. The analyzing of risk may comprise selecting risk features, determining a likelihood of exposure, and/or determining a likelihood of event reversibility. The fuzzy set membership rules may define a risk prevention continuum. The analyzing of risk may be location dependent, age dependent, prior infection dependent (e.g., MRSA, CDI), patient antibiotic exposure dependent, prior medical history dependent, etc. The assessing of resilience may comprise determining a risk management inventory and/or determining a risk avoidance resource. The fuzzy set membership rules may define a risk prevention continuum. The hospital acquired infection risk factor prevention may comprise providing antibacterial surfaces, patient education and discharge planning, and/or a multi-modal strategy.

The hospital acquired infection risk factor prevention may be dependent on at least a cost-effectiveness analysis and/or on a patient safety analysis. The risk of hospital acquired infections may be analyzed with respect to at least risk event identification, risk mitigation, and risk prevention. The performance of the hospital may be inferred based on the fuzzy membership set rules and contextual infection data. The resilience may be assessed with respect to an ability of a hospital to anticipate, avoid, and manage hospital acquired infections. The method may further comprise altering a hospital strategy for managing risk of hospital acquired infections based on the inferred performance. Analysis of the risk of hospital acquired infections may comprises use of fuzzy cognitive mapping to increase reliability and validity of risk mitigation strategies. Analyzing of the risk of hospital acquired infections may comprise use of fuzzy cognitive mapping to assess a stability of a risk event. Analysis of the risk of hospital acquired infections may comprise use of fuzzy cognitive mapping to assess a reversibility of a risk event.

It is also an objet to provide a method for assessing hospital acquired infection risk, comprising: determining resilience inference fuzzy membership categories based on at least fuzzy risk capacity, resilience capacity, and performance safety, as a basis for fuzzy inference system rules; receiving information about a hospital acquired infection risk and hospital acquired infection resilience membership function parameters; and employing a fuzzy inference system dependent on the fuzzy inference system rules and the receiving information to predict specific hospital acquired performance safety outcomes.

The hospital acquired infection risk may be derived though machine learning and heuristics, through machine learning and fuzzy, cognitive mapping, or through at least machine learning to increase a reliability of determination of risk event occurrence. The hospital acquired infection risk may be derived through at least one of heuristics or fuzzy cognitive mapping increase a validity of at least one risk mitigation strategy. The method may further comprise using fuzzy cognitive mapping to assess a stability of a hospital acquired infection risk event. The method may further comprise using fuzzy cognitive mapping to assess a reversibility of a hospital acquired infection risk event. The method may further comprise altering at least one hospital acquired infection risk membership function parameter dependent on at least the predicted specific hospital acquired performance safety outcomes, and/or altering at least one hospital acquired infection resilience membership function parameter dependent on at least the predicted specific hospital acquired performance safety outcomes.

The determining resilience inference fuzzy membership categories may be based on at least fuzzy risk capacity, resilience capacity, and performance safety, as a basis for fuzzy inference system rules comprises supervised learning on labelled data. The labelled data may be labelled for at least one of geography, patient age, patient prior antibiotic use, patient history of MRSA, and patient history of C. diff. The method may further comprise altering at least one hospital facility dependent and/or patient-specific care based on at least the predicted specific hospital acquired performance safety outcomes, a cost-effectiveness analysis, and/or a patient preadmission environmental risk.

### BRIEF DESCRIPTION OF THE FIGURES

FIG. 1 shows resilience inference map of objectives.

FIG. 2 shows resilience markers framework. (adapted from source: Furniss et al., 2011).

FIG. 3 shows integration of resilience markers, resilience assessment and four concepts of resilience response behaviors frameworks into resilience inference model.

FIG. 4 shows resilient systems inference evaluation process framework.

FIG. 5 shown process steps for risk analysis phase.

FIG. 6 shows health systems HAI risk prevention capacity μ_{F}(X).

FIG. 7 shows process steps for resilience assessment phase.

FIG. 8 shows health systems HAI risk prevention capabilities μ_{F}(X).

FIG. 9 shows process steps for performance inference phase.

FIG. 10 shows health systems performance safety potential μ_{F}(X).

FIG. 11 shows HAI risk event evaluation process.

FIGS. 12-19 show correlation plots between CDI and population; MRSA and population; MRSA and density; MRSA and crowding; MRSA and homelessness; and MRSA and MUP; CDI and MUP; and CDI and Rural, respectively.

FIG. 20 shows violin plot of the regional population amounts.

FIG. 21 shows distribution of trimmed U.S. regions.

FIG. 22 shows pairwise regional comparison graph of population amount over 65 years and CDI.

FIG. 23 shows pairwise regional comparison graph of CDI and MRSA.

FIG. 24 shows HAI risk mitigation evaluation process.

FIG. 25 shows HAI resilience assessment process.

FIG. 26 shows risk μ_{F}(X): age above 65 and CDI; resilience μ_{F}(X): copper in healthcare EOC finishes.

FIG. 27 shows risk μ_{F}(X): geographical region and CDI; resilience μ_{F}(X): panarchy of operational prevention.

FIG. 28 shows Risk μ_{F}(X): geographical region and MRSA; resilience μ_{F}(X):
decolonization regimen post discharge.

FIG. 29 shows risk μ_{F}(X): MRSA and CDI; resilience μ_{F}(X): panarchy of operational prevention.

FIG. 30 shows risk μ_{F}(X): CDI and MRSA; resilience μ_{F}(X): clinical feedback standard operating procedure.

FIG. 31 shows HAI performance safety inference process.

FIG. 32 shows performance safety FIS outcome for risk μ_{F}(X): age above 65 and CDI;
resilience μ_{F}(X): copper in healthcare EOC finishes.

FIG. 33 shows performance safety FIS outcome for risk μ_{F}(X): geographical region and CDI; resilience μ_{F}(X): panarchy of operational prevention.

FIG. 34 shows performance safety FIS outcome for risk μ_{F}(X): geographical region and

MRSA; resilience μ_{F}(X): decolonization regimen post discharge.

FIG. 35 shows performance safety FIS outcome for risk μ_{F}(X): MRSA and CDI; resilience μ_{F}(X): panarchy of operational prevention.

FIG. 36 shows performance safety FIS outcome for risk μ_{F}(X): CDI and MRSA; resilience μ_{F}(X): clinical feedback standard operating procedure.

FIG. 37 shows surface map for risk prevention resilience potential and performance safety μ_{F}(X).

FIG. 38 shows an exemplary fuzzy inference system in accordance with embodiments of the present disclosure.

FIG. 39 shows a schematic block diagram of a computing device that can be used to implement various embodiments of the present disclosure.

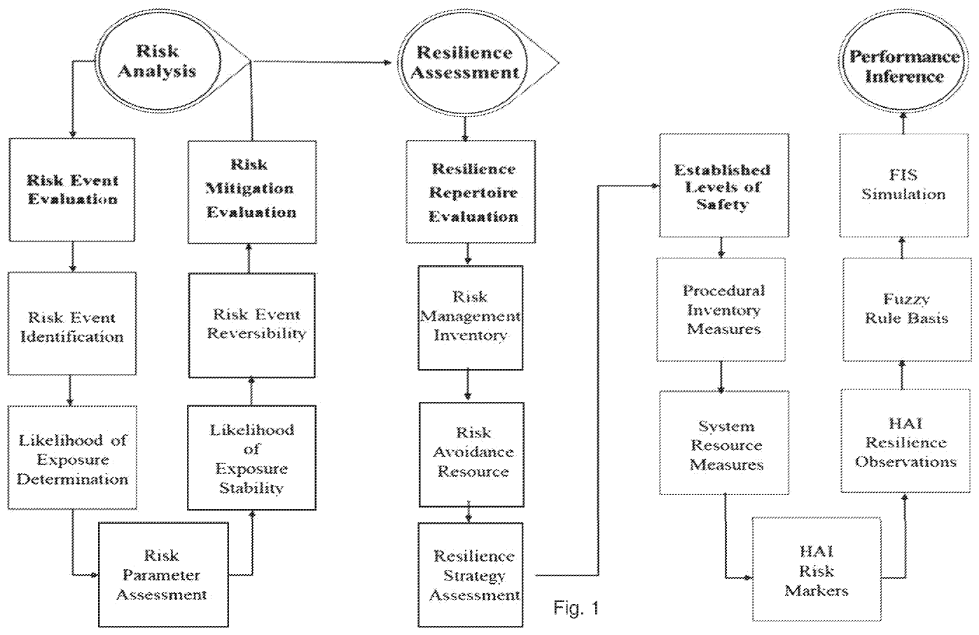

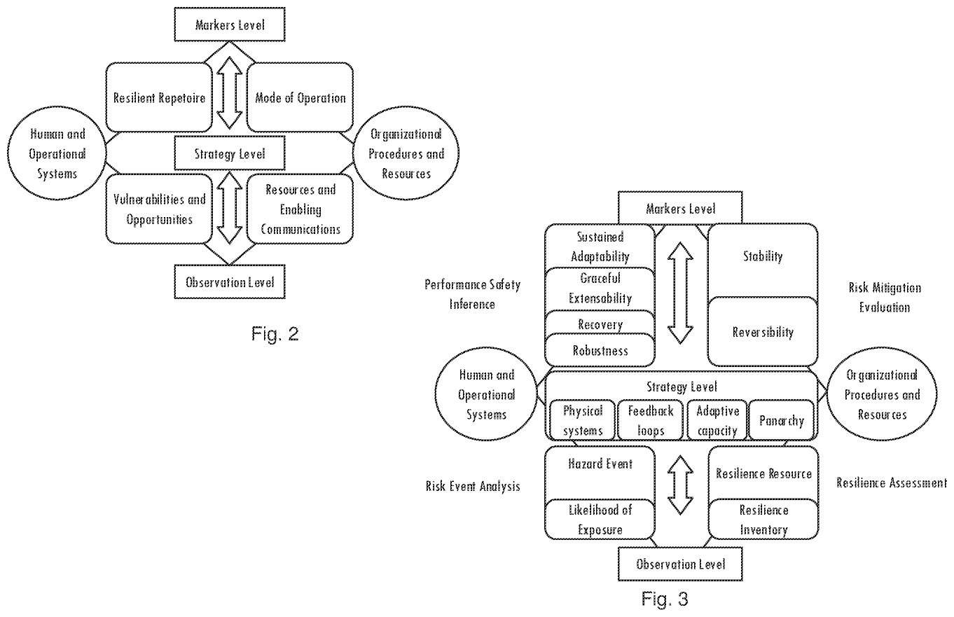

### DETAILED DESCRIPTION OF THE PREFERRED EMBODIMENTS

Due to the increasing complexity of systems in safety-critical organizations like acute care health systems, there is an urgent and growing need for anticipatory rather than reactive operational performance response. The resilience inference methodology demonstrate value in interpreting regional, environmental, and demographic risk and operational resilience factors that are relevant in HAI prevention in acute care environments. This approach to Resilience Inference, which uses both data analysis and fuzzy inference techniques, assists health systems and population health stakeholder groups in understanding factors in potential patient catchment areas that are related to infectivity risks. Like state health safety requirements, regionally specific healthcare design and building codes, and demographically specific population health improvement efforts this information is instrumental in creating more resilient healthcare infrastructure tailored to community safety and risk mitigation priorities.

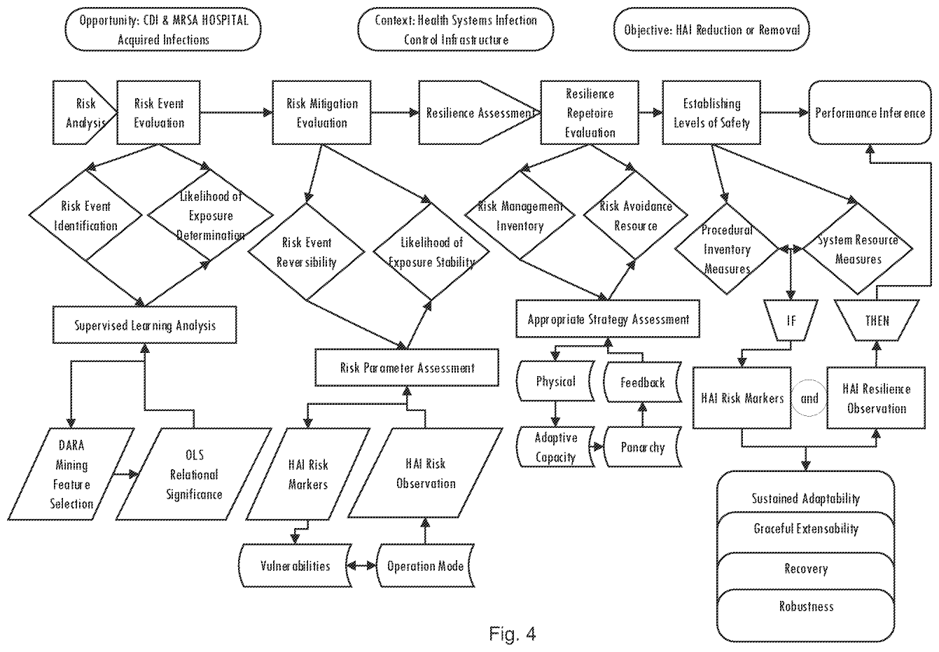

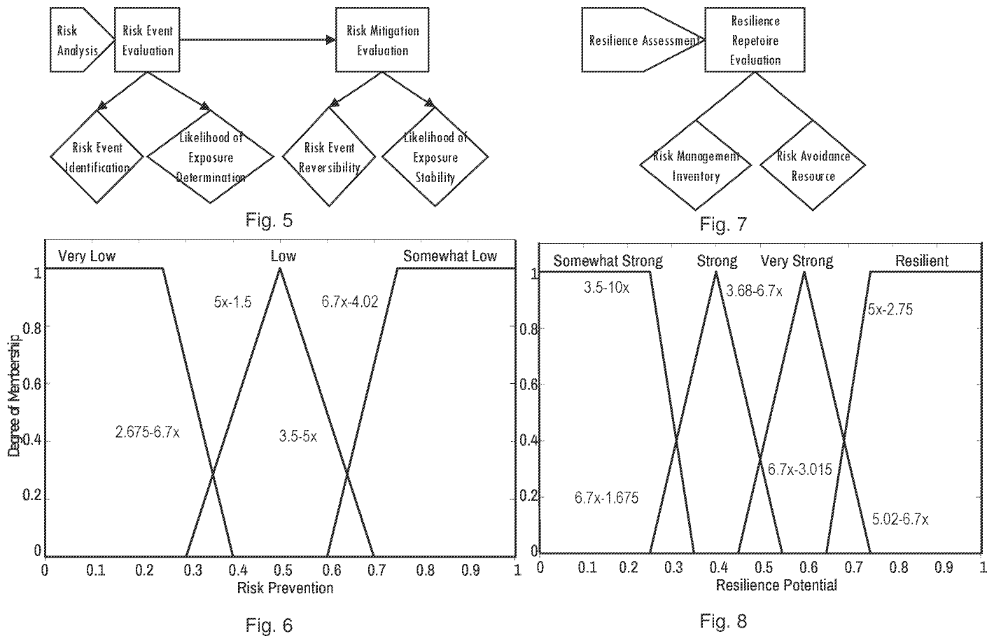

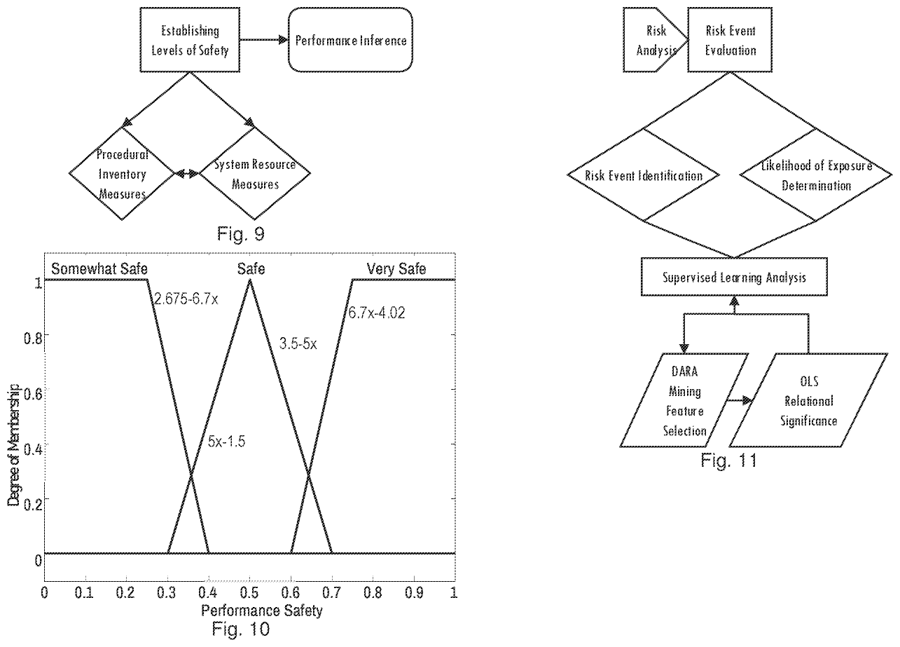

The present technology includes: Risk Analysis: using supervised learning techniques on regional data, and Fuzzy Logic to operationalize the combined processes of: Risk Event Analysis (processes of Risk Event Identification and System Likelihood of Hazard Exposure Determination); Risk Mitigation Evaluation (Risk Parameter Assessment, Likelihood of Risk Exposure Stability, and Risk Event Reversibility); and Risk Prevention Capacity (attribution of the level of system HAI hazard prevention capacity based on Risk Event exposure and Risk Mitigation opportunity); Resilience Assessment: how to measure the Resilience Repertoire of a system which includes a system's Risk Management Inventory, Risk Avoidance Resources, and Resilience Strategy Assessment based on their observed HAI risk moderation performance level across a continuum of Fuzzy Membership sets; and Performance Inference: the process for evaluating the impact of resilient procedural inventory and system resource performance capability based on regional health system HAI risk factor prevention capacity using applied Fuzzy Inference Systems. An illustration of the Resilience Inference methodology process explanation segments is delineated in FIG. 4, 30 which shows a Resilient Systems Inference Evaluation Process Framework. Various endogenous and exogenous factors impact a system's performance with respect to development of a resilient design for almost any hazardous context (Hollnagel et al. 2006). Resilience assessment serves as a complementary tool to extend traditional risk management. Resilience assessment interprets how remediation and adaptation methods can be integrated into system operations to ensure essential systems and critical services are maintained in the face of disruption (Sikula, 2015). Resilient response in the specific setting of engineered systems performance demonstrates a system's successful adaptive capabilities to unplanned high-risk circumstances (Hollnagel et al., 2006). Therefore, resilience assessment estimates how mitigative or adaptive methods can either avert or manage risk response. FIG. 5 shows a Process Steps for the Risk Analysis Phase.

Mathematically derived predictive frameworks may be defined to identify the boundaries of system risk and its impact on system resilience behavior, and test for potential origins and types of hazardous events or risk factors that can then facilitate experimental comparisons of system resilience interventions (Iran et al., 2017). In the scenario of understanding HAI risk, a macro-ergonomic, perspective situates systems rules and procedures in a broader operational context (Carayon et al. 2013). This approach analyzes which variable groupings external to health systems scope of control may have the most significant impact on overall safety outcomes in health system infection control. The resultant information is then be used to guide health system operational process or policy decisions that relate to safer care delivery.

The process of analyzing system risk is usually expressed as an index that involves the quantification of the components of hazard exposure effect. (Linkov et al. 2014). Its use is to understand what external community infectivity hazards factors impact internal hospital infection exposure and how these relate to CDI and MRSA HAI effects. This formula can be expressed within the Risk Analysis framework to ascertain specific health system resources HAI Risk Mitigation capacity. The results of system capacity calculations are a first step in evaluating the potential impacts infectivity risk reduction interventions may have on health systems' resiliency to HAI. For this analysis representative numerical approximations based on High Stability, Moderate Stability, and Low Stability as well as Easily Reversible, Somewhat Reversible, and Difficult to Reverse can be used to define condition stability and condition reversibility. An illustration of this approach as it applies to CDI and MRSA HAI is delineated Table 1.

TABLE 1
Risk Analysis operationalization framework

| Risk Event Evaluation | | Risk Mitigation Evaluation | | |
|---|---|---|---|---|
| Hazard Event | Likelihood of exposure | Condition Stability | Condition Reversibility | Risk Prevention Capacity |
| CDI HAI | Somewhat High | High Stability | Easily Reversible | Somewhat Low |
| MRSA HAI | High | Moderate Stability | Somewhat Reversible | Low |
| | Very High | Low Stability | Difficult to Reverse | Very Low |

Often a comprehensive and systematic evaluation of risk factors reveals that safety-critical systems must demonstrate the ability to operate effectively and with performance continuity outside its formal design-basis (Furniss et al. 2010).

Supervised Learning data analysis techniques manifest relationships between variables that are sometimes perceived as disparate within complex systems. Such an approach offers better ability to calculate both emergent areas of risk as well as the efficacy of responses to potential infection hazard and risk. The process of Supervised Learning analysis can be applied retrospectively on large repositories of readily available data in an automated manner. The application of Fuzzy Logic to systems control specializes in dealing with uncertainty and imprecision. In the context of engineered systems, differences in system performance abilities can be represented through differing fuzzy membership functions relevant to risk mitigation and resilience, attributes critical to each structural element (Muter, 2012).

Fuzzy logic is used in concert with resilience assessment techniques by using linguistic terms to rank both a risk prevention and avoidance capacity: A risk mitigation framework may be constructed that draws on systems subject matter expertise to understand the means and setting of system operation. Modes of system operation in sociotechnical contexts, like healthcare delivery, can be described in part by condition stability and reversibility (Sikula et al., 2015). Using the qualitative information derived from the system expert's subject knowledge to understand system contextual performance is often vague and nuanced in its interpretation. Work that has proposed an applied fuzzy theory to apportion risk categorization has proven effective in introducing a way to more easily quantify risk levels defined through linguistic variables and thus measure subjectively defined performance attributes (Chang & Cheng, 2010). One strength of fuzzy logic is that it is able to draw in semantic expressions of experts to derive operable rules. An example of HAI Risk Prevention capacity levels can be described along a continuum of representative Triangular Fuzzy Number (TFN) and Trapezoidal Fuzzy Numbers (TrFN) that define membership categories as illustrated by FIG. 6, which shows Health Systems HAI Risk Prevention Capacity μF (X).

There are validated advantages of tapping into specific areas of SME to define the boundaries of fuzzy probabilistic risk and adaptive response to hazard models for the analysis of the interaction between human activity and socio-technical systems (Konstandinidou et al., 2006; Hollnagel, 1998). Furthermore, fuzzy categories of risk defined by natural language along a multidimensional scale that considers degrees of likelihood and conditions of occurrence offer a way to introduce greater quantitative rigor in hazard occurrence prediction. Integrating least square and statistically derived feature selection ranking methods offer the potential for generating more accurate fuzzy classifications and greater robustness against analysis uncertainty (Zhang & Chu, 2011).

Assumptions that risk categorizations are equally weighted can lead to an oversimplification of system abilities and incorrect inferences regarding system risk and performance safety (Chang & Cheng, 2010). Employing statistical methods such as Ordinary Least Squares (OLS) improves the accuracy of analysis model variable matching and the likelihood of relational outcomes by comparison of overall system fit. OLS has demonstrated suitability in offering higher specificity to the weighting of different system risk categories (Cheung, 2007). It also has well-established precedence of being used in comparing overall historical patterns, in health systems risk analysis (Fuller et al. 2016). Used iteratively and continuously validated augmented risk management techniques can offer a viable pathway for system resilience building (Sikula et al., 2015). This extension of Risk Analysis by using Supervised Learning, OLS likelihood metrics and Fuzzy Logic for hazard identification, exposure, effect partitioning, and weighting, and risk mitigation capacity potential lays the foundation for infection prevention strategy development and resilience efficacy testing.

Data Sources: Separate data on regional population demographical characteristics such as population by states that were over the age of sixty-five (65), state population density; and urban and rural designated proportion by U.S. state were accessed and downloaded. Additionally, living conditions that necessitate more than one-person occupying a room on a full-time basis are designated as “over-crowded” by the U.S. Department of Housing and Urban Development (Blake et al. 2007). This specific aspect of population density was considered necessary since community survey data on overcrowding in housing has been specifically associated with increased incidences in community-onset MRSA (Immergluck et al., 2019; See et at 2017). Datasets available from USICH on the number of homeless adults in 2017 were accessed and downloaded. Additionally, HRSA's portal on medically underserved health service areas (MUA) and medically underserved population (MUP) were also downloaded.

According to the HRSA, the difference between these two designations is that MUAs are identified as regions that have a shortage of primary care health services for residents within a geographic area, such as (Description of “Medically Underserved Areas and Populations” (MUA/Ps): HRSA, 2016): a whole county; a group of neighboring counties; a group of urban census tracts; and a group of counties or civil divisions. MUPs are related to MUA but are representative of persons rather than geographic areas. MUP represents a count of specific sub-groups of people living in a defined geographic area with a shortage of primary care health services. These groups may also be identified as particular populations that struggle with economic, cultural, or linguistic barriers to health care. Examples include, but are not limited to, those who are (Description of “Medically Underserved Areas and Populations” (MUA/Ps): HRSA, 2016): homeless; low-income; Medicaid-eligible; Native American; and migrant farmworkers.

This data was then organized into subsets as delineated by U.S. Census regions. These Census groupings subdivide the continental U.S. and the states of Alaska, Hawaii and the District of Columbia into four (4) geographic areas for presentation of population census data. These regions are demarcate) as follows (Mackun, et al. 2011): North East (NE); South (SE) (Includes Washington D.C.); Midwest (MW); and West (WE) (includes Alaska and Hawaii). This effort was made to balance the population and to ensure regional data, as opposed to U.S. state-based data, was the geographic factor driving the analysis. For example, individual states such as New York and California have more numerous residents in general and cities with higher population densities than other states within their geographic regions such as Maine and Idaho. Additionally, states with geographies like Alaska are designated as almost 100% “Rural,” and Washington D.C. is designated as 100% urban.

CDC's National Healthcare Safety Network (NHSN) data on State-specific observed HAI incidence rates in hospital settings was also downloaded. It was determined that data to be used for this analysis are constrained to only the most current inpatient observed amounts of CDI and MRSA HAI that had been documented within a single year (e.g., 2017). Furthermore, since Acute Care Hospitals had the highest percentage and most consistent health system reporting the decision was also made to constrain data to this environment of care setting. The United States Virgin Islands, and territories of Puerto Rico and Guam were not included due to the lack of available reported data on observations of hospital-onset CDI and MRSA in acute care settings.

Data Analysis: Supervised learning methods can be used to make predictions about variables with known outcomes when the specific values of input variables are also identifiable (Obenshain, 2004), in this study, the outcome or dependent variables are known and indicated in the CDC data. Namely: Hospital-onset CDI specific incidence rates in acute care settings; Hospital-onset MRSA specific incidence rates in acute care settings. Furthermore, the values of the input or independent variables were also indicated in the data, which included the data subset categories of: Regional Population; Population over the age of 65; Number of Homeless Adults; People per Square Mile (i.e., Population Density); Number of People living in crowded homes; Medically Underserved Areas (MUA) by region; Medically Underserved Populations (MUP) by region; Amount of region designated as “Rural”; and Amount of region designated as “Urban”.

In Supervised Learning analysis procedures and observed outputs are part of the training data that augments reliability in testing outputs (James et al, 2013). Of the Supervised Learning techniques used in conjunction with Fuzzy Logic, OLS is both robust and mathematically well established (Guillaume, 2001). This method has also proven useful in selecting the most influential factors and firing strengths for input membership functions which have also helped guide Fuzzy Inference System Rule formation (Cheung, 2007; Destercke et al. 2007).

A critical aspect of ensuring efficiency and accuracy of Supervised Learning techniques is feature selection (James et al., 2013). Both data preprocessing, feature selection and parameter tuning or finding the best combination of parameters have a significant impact on data analysis performance that can surpass the importance of the actual choice of the analysis outcome classification model (Ehrentraut et al., 2012). Feature selection in the data for membership weights and to inform fuzzy rule formation was done by using “Pearson's r” for determining significant correlations between CDI and MRSA HAI and independent regional geographic and demographic data subsets. This method has proven suitable for feature selection in the research of supervised learning outcomes reliability. Pearson's r as a filter method for data preprocessing, estimate generalization, and to remove irrelevant attributes before induction occurs, has also demonstrated the improvement of function validity (Weston et al., 2001).

For certain types of characteristics, even vast quantities of data representative of large populations are frequently not perfectly normally distributed, which was the case with much of the open-source data obtained as described above. The benefit of using OLS for data analysis is that standard proof of the unbiasedness of its estimates does not require the assumption of data distribution normality or constant variance (Cheung, 2007). According to research on OLS regression techniques, one reason these methods are ubiquitous in their use is because of them being more robust than other methods of statistical analysis against violations of normality and providing unbiased, efficient and consistent estimators in most situations (Habeck & Brickman, 2018). When data is not normally distributed, the mean of the analysis outcome may or may not be a goal measure of central tendency but maybe a suitable indicator of the proportion of risk and representative risk differences between variables (Cheung, 2007). This approach to analyzing RAI risk data could provide a viable way of interpreting CDI and MRSA Risk Prevention capacity. The weighting of Risk Event potential outcomes using the information provided as a result of applied. OLS regression therefore improves the accuracy of insight into the fuzzy levels of Risk Prevention capacity based on the estimated significance of relationships between dependent and independent variables.

Reframing Risk Analysis Hypotheses as Fuzzy Inference Rules: Orthogonal transformation methods can also provide a vehicle for budding Fuzzy Inference System rules from a limited subset of data relationships deemed to be statistically meaningful. OLS has been used to create rules from a set of training data by selecting those most important though linear regression techniques (Destercke et al., 2007). The generation of rule formation is a critical component in the development of Fuzzy Inference System informed strategies to mobilize resilient response goals. This rule appears to be especially true in circumstances where human sentience plays a critical role in systems resilience potential such as healthcare (Anooj, 2012; Leite et al., 2011). In using a Fuzzy Inference System for the development of performance safety estimation for infection control, it is helpful to establish MAX and MIN vectors that serve as the basis for decision rules that can specify the risk and resilience level for health system infrastructure based on a set of numerical variables. These decision support rules can aid system analysts in more accurately diagnosing and mitigating risk factors and integrating reliable resilience resources for moderating the effects of risk (Anooj, 2012).

Reframing the Risk Analysis hypotheses as Fuzzy IF-THEN rules, for example, based on the information obtained in the comprehensive literature review regarding CDI and MRSA risk factors, hypotheses derived membership categories are as follows (dependent and independent variables have been italicized): IF region Population numbers are large, THEN both CDI and MRSA HAI Likelihood of Exposure is High; IF region Population numbers above 65 years old are great, THEN CDI HAI Likelihood of Exposure is High; IF region Homeless Populations are large, THEN MRSA HAI Likelihood of Exposure is High; IF regional area Population Density Proportion is high, THEN MRSA HAI Likelihood of Exposure is High; IF regional area Household Crowding Proportion is high, THEN MRSA HAI Likelihood of Exposure is High; IF region MUA Amount is large, THEN CDI and MRSA HAI Likelihood of Exposure is High; IF region MUP Amount is large, THEN CDI and MRSA HAI Likelihood of Exposure is High; IF region Rural Designated Area is large, THEN CDI HAI Likelihood of Exposure is High; IF region Urban Designated Area is large, THEN MRSA HAI Likelihood of Exposure is High; IF regional Geographical Location is in a certain area in the continental U.S., THEN CDI or MRSA HAI Likelihood of Exposure is High; IF regional MRSA HAI incidence rates are high, THEN CDI HAI Likelihood of Exposure is High; and IF regional CDI HAI incidence rates are high, THEN MRSA HAI Likelihood of Exposure is High.

Resilience Assessment: Incidences of using statistical analysis and fuzzy logic for analyzing risk is extended to evaluate systems' adaptive capacity performance. System resilience assessment can also employ the use of fuzzy linguistic variables to express the relative importance of identifiable operational weighted resilience factors in a broader systemic context to identify the relational synergies between system elements (Tadić, et al., 2014). FIG. 7 shows a Process Steps for Resilience Assessment Phase.

The linguistic variables that define the performance qualities of resilient systems are both vague and context-specific (Dubois, 1980); in other words, resilience capability measures could in and of themselves be considered Fuzzy Sets. Furthermore, system resilience technical performance variables could also be reasonably linked to the fuzzy constructs of plausibility in performance reliability and belief in systems fitness for compensatory procedures. These conditions create a need for considering the possibility for system adaptive capacity and necessity for active resilience (Klir & Yuan, 1995). This occasion exemplifies both the opportunity and challenge for preemptively identifying and developing ways to measure the effects of resilience response concurrently to defined risk prevention capacity.

The same approach to defining Risk Prevention capacity categories of Somewhat Low, Low, and Very Low can be generalized into Resilience Potential capability. Using the nested groups that define the performance of a system's resilience repertoire lend themselves almost naturally to this effort. Each of these characteristics can be translated into fuzzy membership functions for system risk response capabilities to external variable impact. The memberships for these resilience nested attributes can also be represented by a combination of TFN membership functions (i.e., T-norms), and TrFN membership function (T-conorms).

The same approach can also be used to construct overlapping the membership categories of performance output variables that can be aggregated to define a specific resilience output capability based on fuzzy input variables. These categories are grouped by their perceived percentage level of systems resilience performance, and then apportioned into associated resilience performance membership functions [μF(x)]. FIG. 8 shows the Health Systems HAI Risk Prevention Capabilities μF(X). Resilience potential capability levels can be applied within the investigative context of estimating hospital-acquired infection prevention infrastructure adaptive response and categorized according to the Resilience Assessment Markers Model “Strategy Level” categories of Physical Systems; Feedback Loops; Adaptive Capacity; and Panarchy. This taxonomy offers a framework for evaluating the measurable resilience capability potential of different types of system strategy interventions. Furthermore, it provides a way to assign specific resilience augmenting markers to a particular area of defined risk.

An obstacle to reliably evaluating system response resilience is the current deficit of data related to this area. This barrier is especially true for HAI incidence “lessons learned.” This issue is because processes that enable organizationally specific HAI incidence rate improvement or information regarding known internal failure point moderation within systems are rarely if ever made public. However, there is a burgeoning number of discrete case study research articles that can be mined for this purpose. This data on resilience performance perhaps lacks the external validity and accuracy of the supervised learning approaches that can be applied to risk-related information contained in vast publicly available data repositories. However, it does present a way to capture and use HAI event-specific improvement metrics and consider them within a broader framework of infection prevention operational resilience within health systems. Aggregated rules reliant on risk mitigation and resilience capability level allow for the construction of a set of conjunctive system of rules. Safety potential to infer system performance safety is inferred where both conditions are jointly satisfied (Ross, 2009).

Evaluating HAI resilience in healthcare settings often presents the need to estimate systems performance in the context of vague and stochastic circumstances such as infection control “Safety” and healthcare environment “Infectivity” level. Using case study derived performance data, resilience capability potential is assigned based on a comparison of the effectiveness of HAI impact reduction strategies. Speculative performance data could then be predicted using Fuzzy Logic membership functions as a basis for parametric evaluation of a consequent resilience metric from a given case study with an associated fuzzy membership set metric. Classification of specific resilience interventions may be organized according to the strategy level categories outlined in the Resilience Inference Model. This develops insight into what types of health systems infrastructure enhancements may offer the best performance safety outcomes related to specific HAI (e.g., CDI, MRSA, or both). It also helps to better understand the level of effort and investment a particular type of HAI resilience intervention required for achieving a certain level of infection prevention performance safety.

More generally, the present invention provides a paradigm for use of uncontrolled or non-experimental data, social science literature, and other expert or scholarly literature, in controlling investments and policies derived from implication rather than proven cause and effect. While the particular data employed herein relates to HAI, and the results applied in that arena, the methodology is not so limited, and rather exploits the resilience inference model.

An example of how fuzzy resilience membership level assignment is assigned based on HAI reduction performance derived from case studies is outlined in Table 2.

TABLE 2
Quantified HAI Resilience Potential Capability

| | | | HAI | |
|---|---|---|---|---|
| Resilience Strategy | HAI | | Reduction | Resilience |
| Level Theme | Relevance | Strategy Employed | Outcome | Potential |
| Physical | CDI | Self-disinfecting copper | 78% | Resilient |
| Systems | | | reduction in | |
| | | | CDI | |
| Predictive or | MRSA | Feedback about related | 31% | Somewhat |
| Preventative | | preventable factors | reduction in | Strong/Strong |
| Feedback | | | MRSA | |
| Adaptive | MRSA | Post-discharge hygiene plus | 30% | Somewhat |
| Response or | | decolonization regimen training | reduction in | Strong/Strong |
| Capacity | | | MRSA | |
| Risk Prevention | CDI | Multidisciplinary HAI reduction | 42.7% | Strong |
| Panarchy | | oversight of clinical & | reduction in | |
| | | environmental operations & | CDI | |
| | | hand | | |

In the application of resilience engineering analysis and strategy formation, the management of membership function values is facilitated through the integration of fuzzy set theory. The construction and comparison of conjoined membership functions lead to the generation of a set of fuzzy rules (Anooj, 2012). This proposed method is predicated on the building of fuzzy modalities, which allows for the creation of fuzzy values from a predefined set of quantitative controls. The parameters of each of the membership function category are defined to determine HAI Performance safety. The Risk Prevention classification uses the Resilience Inference model as a guide and is based on hazard event parametric quantification as wet as being intrinsically linked to a mitigation mode of operations. The resilience potential levels are derived from the evidence-based performance capabilities previously explained as they relate to specific HAI reduction. Using the Risk and Resilience fuzzy membership levels allow inputting these two disjoint functions into an analysis software platform capable of computing and running multiple tests on variable input and output combinations. The numerical parameters for each of the Resilience Inference component membership categories are diagramed in Table 3.

TABLE 3
Resilience Inference Fuzzy Membership Categories

| | Risk Prevention | | Resilience Potential | | Performance Safety | |
|---|---|---|---|---|---|---|
| OLS Level | μF(x) | | μ_{F}(x) | | μ_{F}(x) | |
| p < .05 | Somewhat Low | 0.60-1.0 | Resilient | .65-1.00 | Very Safe | 0.60-1.0 |
| p < .01 | Low | 0.30-.70 | Very Strong | .45-.75 | Safe | 0.30-.70 |
| p < .001 | Very Low | 0-.40 | Strong | .25-.55 | Somewhat Safe | 0.00-.40 |
| | | | Somewhat Strong | 0-.35 | | |

TABLE 4
Fuzzy Rule Basis for Resilience Inference
and HAI Performance Safety

| | |
|---|---|
| 1 | IF Risk Prevention is Very Low AND Resilience is Somewhat Strong |
| | THEN Performance Safety is Somewhat Safe. |
| 2. | IF Risk Prevention is Very Low AND Resilience is Strong THEN |
| | Performance Safety is Safe. |
| 3. | IF Risk Prevention is Very Low AND Resilience is Very Strong |
| | THEN Performance Safety is Safe. |
| 4. | IF Risk Prevention is Very Low AND Resilience is Resilient THEN |
| | Performance Safety is Safe. |
| 5. | IF Risk Prevention is Low AND Resilience is Somewhat Strong |
| | THEN Performance Safety is Safe |
| 6. | IF Risk Prevention is Low AND Resilience is Strong THEN |
| | Performance Safety is Safe. |
| 7. | IF Risk Prevention is Low AND Resilience is Very Strong THEN |
| | Performance Safety is Very Safe. |
| 8. | IF Risk Prevention is Low AND Resilience is Resilient THEN |
| | Performance Safety is Very Safe. |
| 9. | IF Risk Prevention is Somewhat Low AND Resilience is Somewhat |
| | Strong THEN Performance Safety is Safe. |
| 10. | IF Risk Prevention is Somewhat Low AND Resilience is Strong |
| | THEN Performance Safety is Very Safe. |
| 11. | IF Risk Prevention is Somewhat Low AND Resilience is Very Strong |
| | THEN Performance Safety is Very Safe. |
| 12. | IF Risk Prevention is Somewhat Low AND Resilience is Resilient |
| | THEN Performance Safety is Very Safe. |

The potential efficacy of HAI resilience interventions on estimated HAI risk prevention levels are analyzed in order to estimate HAI performance safety outcomes. Fuzzy Inference Systems provide insight into the potential expected safety outcomes based on Risk and Resilience inputs: The risk analysis may be extended to assertions based on combined inference to create the foundational rule basis that the Fuzzy Inference System uses for defuzzifying inputs to create a crisp output of estimated safety. However, these rules are meant for HAI safety inferential purposes only. For actual validation of resilience, real-world testing with a control group is used to determine safety outcomes.

The analysis approach combines the use of supervised learning data analysis and Fuzzy Logic and Fuzzy Inference Systems as the primary methods used in operationalizing a HAI Resilience Inference methodology. The selection of these methods was based on their precedent combined utilization for risk analysis and increasing use in extending Resilience Assessment frameworks. Analysis included observations of CDI, and MRSA HAI in acute care settings in the U.S. CDI and MRSA observed HAI in acute care settings were selected because of their escalating risk prevalence and AMR concerns, documented impact on hospital reimbursement, as well as the availability of third-party validated (e.g., CDC) incidence data.

The sourcing of data considered date alignment for information whenever possible. Careful observation of this practice was engaged for fluctuating data with the real potential to change significantly on an annual basis. Additionally, even though OLS regression does not specifically require it, the removal of outliers from independent variable sets was performed to improve distribution normality and ultimately outcome inference generalizability. Although this reduced the final number of observations that is used for training and testing data in the OLS, it was a factor to address for Pearson's r feature selection and was therefore also carried through to regression to determine variable relationship strengths. The increasing of distribution normality was also the rationale for the grouping of independent variables by region as opposed to states.

The system tools used for data analysis feature selection and regression was performed on Python Anaconda Navigator in Jupyter Notebooks using the following import modules: Seaborn; dumpy; Matplotlib.pyplot; Pandas; Statistics; Scipy.stats: norm; Sklearn: LabelEncoder; LinearRegression; StandardScaler; and Statsmodels. MATLAB R2019a Fuzzy Logic Designer toolbox was used for the development of fuzzy membership parameters for all Resilience Inference input and output components. This system was also used for the integration of all fuzzy inference system rules and simulated outcomes.

The rising demand for increased accountability in safety-critical system performance in many high-risk industries predicates the necessity for improved reliability of systems' performance safety. This potential growth trajectory will be aided greatly by contextual resources such as machine learning which could help to facilitate this method of predictive analysis and make it more efficient to apply (Anooj, 2012).

An applied supervised learning and fuzzy inference approach is used for the evaluation of readily available and open-source data. Information on U.S. based-regional population, demographic, and healthcare access data is compared to national incident reporting on HAI caused by MRSA and C. difficile bacteria. The analysis suggests that meaningful relationships between U.S.-based geographic risk factors and dangerous pathogens causing HAI can be derived using these methods. Analyzing regional demographic and environmental data could aid U.S. health systems in more effectively predicting and thus proactively preventing the incidence of HAI in their patient populations in an acute environment of care setting.

Data Preprocessing for Analysis Preparation: Organization of data in comma-separated value (CSV) files, as well as a preliminary review and preprocessing of data distribution, was performed using Python data analysis for U.S. state and region. State population numbers were used as a baseline for HAI incidence rates analysis. U.S. geographic regions are defined by the states that comprise them, and more people per region are directly related to more observed incidences of CDI and MRSA HAI. An evidence-based intuitionistic “Risk Event” evaluation degree of likelihood matrix was created.

TABLE 5
Risk Event Evaluation Pre-Analysis Likelihood Assignments

| | Risk Likelihood | |
|---|---|---|
| IF/THEN | CDI | MRSA |
| Population | High Likelihood | High Likelihood |
| >65 Years Old | High Likelihood | |
| Homeless | | High Likelihood |
| Density | | High Likelihood |
| Crowding | | High Likelihood |
| MU_HSA | High Likelihood | High Likelihood |
| MUP | High Likelihood | High Likelihood |
| Rural | High Likelihood | |
| Urban | | High Likelihood |
| U.S. Geography | High Likelihood | High Likelihood |
| DII | | High Likelihood |
| MRSA | High Likelihood | |

Table 5 represents the research hypothesis questions rewritten as IF/THEN rules and then organized in a matrixed format. Reframing the research question hypotheses in this manner provides a basis for building up to and validating the items comprising the Fuzzy Rule Basis for Resilience Inference as represented by HAI Performance Safety estimates. This information is indicated in Table 4 through supervised learning analysis methods. However, Table 4 was set up only as an illustration of presumptions before actual quantitative analysis. Likelihood scale assignment was resultant from a review of precedent research because there was no basis for comparison of these types of regional risk event probabilities before the data analysis. Given this scenario, all variables were equally likely at a medium range of a scale of “high” likelihood. These relationships of independent variables to the dependent variables of CDI and MRSA were then updated after each stage of Risk Analysis (e.g., feature selection through Person's r and Ordinarily Least Squares Regression). Categorical numerical codes were assigned to each U.S. Census-defined region in the continental U.S. This was done primarily for consistency and to dictate dummy variable coding assignment for the Ordinarily Least Squares (OLS) regression. The original data for the entire U.S. regional population Mean, Median, and Standard Deviation are as follows (as represented by Total State Population/10,000): U.S. State Population Observations: N=51; μ=638.67; M=445.42; σ=724.47. A distribution plot of the population data of all 50 states and the District of Columbia (N=51) confirmed a positively skewed and high variance distribution of data. Additionally, when viewed as a boxplot diagram by designated U.S. Census regional groupings, the distribution of these subsets of data showed several state-based outliers in populations in the West (4), Southeast (3), and Midwest (2) groups. The Northeastern region (1) data contained no such population outliers.

Plots of other independent variable data also indicated a high degree of skewness and leptokurtic distributions due to the presence of outliers. This information included state-based density variables of people per square mile (i.e., population density) and overcrowding conditions in housing. Additionally, Medically Underserved Areas (MUA) had a positive skew due to regional outliers. Finally, the independent variable of regional areas designated as “Rural,” and “Urban” were highly negatively and positively skewed, respectively. This outcome was unsurprising since according to the U.S. Census Bureau, only 3% of the entire land area of the U.S. is considered “Urban”. Rural designated areas comprise 97% of the geographic area of the U.S., but only 19.3% of the population lives in these areas (Ratcliffe et al., 2016). The individual distributions of independent variables with high levels of skewness and leptokurtic distributions due to the presence of outliers.

Understanding that improving the normality of the data distribution was necessary not only for primarily feature selection but also to augment the accuracy of predicting common regional HAI risk factors, state-based high-density population outliers were removed. Trimming states with very low populations was also done to improve data generalizability. States with much higher than average levels in housing overcrowding and levels of MUA were removed from the regional analysis sample taken from the U.S. population. Finally, trimming states that were almost 100% rural such as Alaska and Wyoming and areas that were 100% urban such as Washington, D.C. was also done to improve data normality. The trimmed data for the U.S. regional population Mean, Median, and Standard Deviation after outliers were removed from all independent variables with highly skewed distributions are as follows (as represented by Total State Population/10,000): Trimmed U.S. State Population Observations: n=30; x̅=507.46; M=456.93; sd=304.03. The trimming of data to improve the overall normality of each variable factor to be considered in the CDI and MRSA HAI risk and resilience relational analysis did considerably reduce the total number of states in the evaluation sample. However, this effort did improve the normality of all independent variable data distributions that were highly skewed when all 50 states and Washington D.C. were considered.

Although the sample of U.S. regions included only 30 states and districts rather than the original 51, improving data normality was imperative for using Pearson's r correlation for feature selection. The aim of selecting features was to include only those that were the most statistically viable. These remaining variables are then used in the OLS regression model that compares CDI and MRSA HAI incident rates with regional and demographic risk factors. Additionally, outcomes from a representational sample of U.S. regions were obtained that offer insight into significant geographic, environmental, and population factors that demonstrated statistically meaningful relationships with CDI and MRSA HAI incident rates in acute care settings. Even feature selection methods that were tenable with highly skewed samples of U.S. data would have eroded the intent of assumption generalizability. FIG. 11 shows the HAI Risk Event Evaluation Process.

It is important to understand apparent relationships that exist between geographic demographics and health access characteristics across the U.S. (e.g., Regional Populations, Rural/Urban Designation Proportion, Populations of comprised of many older adults, Medically-Underserved Areas and Population, et al.) and the number of observations of HAI caused by specific pathogens (e.g., C. diff. and MRSA) in acute care settings to establish an appropriate model for HAI regional risk analysis. A Pearson's r correlation coefficient was computed to assess the relationship between each regional predictor and HAI target variable to evaluate contextual correlations between these factors.

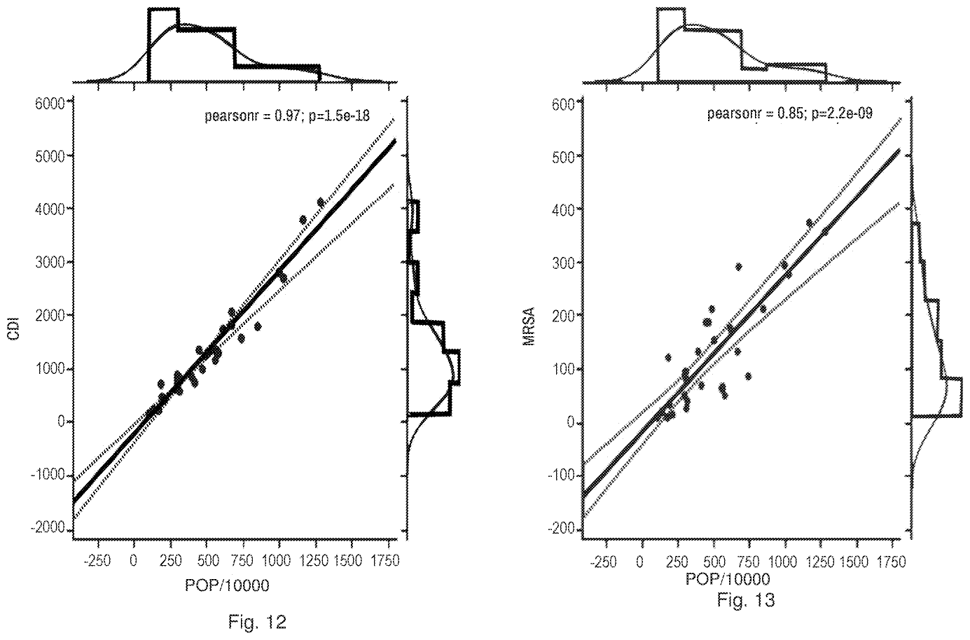

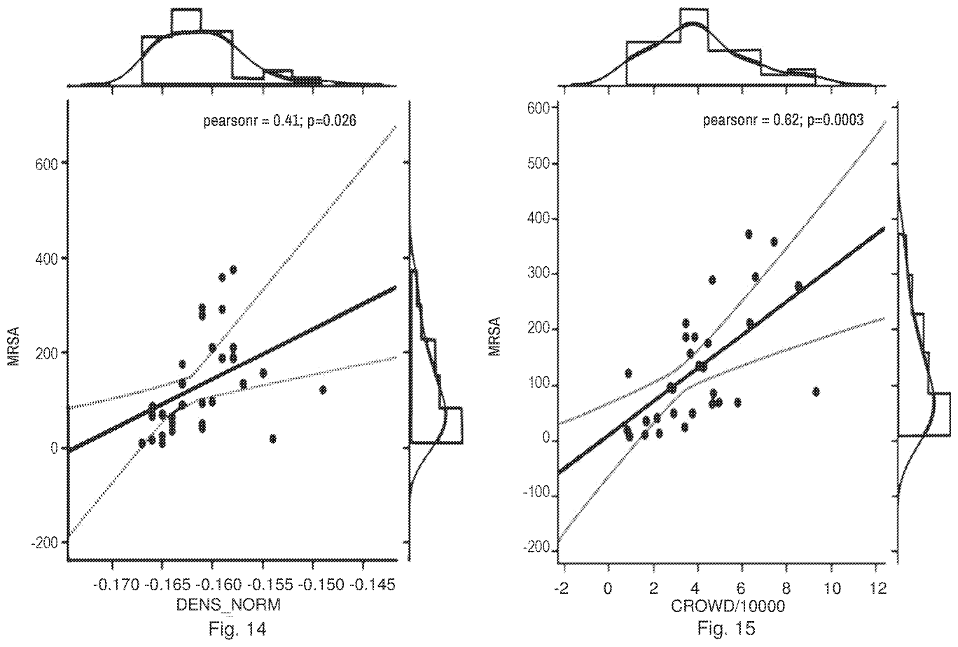

An initial baseline Pearson's Correlation of predictive variable of the regional population was performed to establish a relationship based on the assumption, that more people per collective state-based region is directly related to more people infected by CDI and MRSA HAI did indeed exist. Overall, there was a strong, positive correlation between regional population and both observed CDI and MRSA HAI as indicated in FIGS. 12 (correlation of CDI and Population) and 13 (correlation of MRSA and Population).

The same Pearson's correlation process was used for comparison of the independent variables with the type of HAI (e.g., CDI, MRSA or both) that had been cited in the literature review as potential risk factors for these two specific HAI. These independent variables included the following: Population over the age of 65; Number of Homeless Adults; People per Square Mile (i.e., Population Density); Number of People living in crowded homes; Medically Underserved Areas (MUA) by region; Medically Underserved Populations (MUP) by region; Amount of land area of region designated as “Rural”; and Amount of land area of region designated as “Urban”.

Additionally, CDI incident rates were compared to MRSA rates, and vice versa, to ascertain meaningful relationships between variables. Comparison of the four regions (i.e., Northeast, Midwest, Southeast, and West) was excluded from feature selection, as these are categorical variables and thus inappropriate for a Pearson's r correlation statistical inference method. The results of the Pearson's correlation between dependent and independent variables are indicated in Table 6. The organization of this information has been ordered in a manner that relates these outcomes directly back to the research question hypotheses.

TABLE 6
Pearson's r of relational CDI & MRSA HAI env. and demographic factors

| | | | |
|---|---|---|---|
| Hypothesis | Correlation | r | alpha |

Baseline

| | | | |
|---|---|---|---|
| Higher numbers for a regional population are associated with higher | Y | 0.97 | 1.50E−18 |
| numbers of CDI incidences | | | |
| Higher numbers for a regional population are associated with higher | Y | 0.85 | 2.20E−09 |
| numbers of MRSA incidences | | | |

Question 1: What is the relationship between regional population compositional factors and the risk for AMR
hospital-onset infectivity?

| | | | |
|---|---|---|---|
| Hypothesis 1a: U.S. regions with a larger proportion of their populations | Y | 0.98 | 8.7E−22 |
| over the age of 65 have an increased risk for incidents of hospital-onset | | | |
| CDI. | | | |
| Hypothesis 1b: U.S. regions with higher populations of homeless | Y | 0.39 | 0.032 |
| persons have an increased risk for hospital-onset MRSA. | | | |
| Hypothesis 1c. i.: U.S, regions with higher populations densities have | Y | 0.41 | 0.026 |
| an increased risk for hospital-onset MRSA. | | | |
| Hypothesis 1c. ii: U.S. regions with higher populations of housing over- | Y | 0.62 | 0.00003 |
| crowding have an increased risk for hospital-onset MRSA. | | | |

Question 2: How does healthcare accessibility appear to effect and the risk for AMR hospital-onset infectivity?

| | | | |
|---|---|---|---|
| Hypothesis 2a. i: U.S. regions with more Medically Undeserved Areas | Y | 0.59 | 0.00054 |
| (MUA) have an increased risk for incidents of hospital-onset CDI. | | | |
| Hypothesis 2a. ii: U.S. regions with more Medically Undeserved | Y | 0.36 | 0.048 |
| Populations (MUP) have an increased risk for incidents of hospital- | | | |
| onset CDI. | | | |
| Hypothesis 2b. i: U.S. regions with more Medically Underserved | Y | 0.71 | 2E−05 |
| Areas (MUA) have an increased risk for hospital-onset MRSA. | | | |
| Hypothesis 2b. ii: U.S. regions with more Medically Underserved | Y | 0.37 | 0.043 |
| Populations (MUP) have an increased risk for incidents of hospital- | | | |
| onset MRSA. | | | |

Question 3: How does the rural or urban status of health systems patient catchment area affect the risk for AMR
hospital-onset infectivity?

| | | | |
|---|---|---|---|
| Hypothesis 3a: U.S. regions with more “rural status” defined areas | N | −0.90 | 1.6E−11 |
| have an increased risk for incidents of hospital-onset CDI. | | | |
| Hypothesis 3b: U.S. regions with more “urban status” defined areas | Y | 0.90 | 1.9E−11 |
| have an increased risk for incidents of hospital-onset MRSA | | | |

Question 4: What is the relationship between U.S. geographic region and the risk for AMR hospital-onset
infectivity?

| | | | |
|---|---|---|---|
| Hypothesis 4a: There is a relationship between U.S. regional | NA | NA | NA |
| geographic location and incidents of hospital-onset CDI. | | | |
| Hypothesis 4b: There is a relationship between U.S. regional | NA | NA | NA |
| geographic location and incidents of hospital-onset MRSA | | | |

Question 5: What is the relationship between the co-occurrence of hospital-onset
C. diff. and hospital-onset MRSA in acute care hospitals in U.S. regions?

| | | | |
|---|---|---|---|
| Hypothesis 5a: The incident rates of hospital-onset MRSA are | Y | 0.90 | 1E−11 |
| related to the incident rates of hospital-onset CDI. | | | |
| Hypothesis 5b: The incident rates of hospital-onset CDI are related | Y | 0.90 | 1E−11 |
| to the incident rates of hospital-onset MRSA. | | | |

After preliminary evaluation, it was determined which specific regional factors were the most strongly associated with MRSA and C. diff. HAI occurrence. These results offer insight into factors which elicit the strongest potential as associated predictors for HAI risk from these two-specific antibiotic-resistant bacteria. See, (Roberts et al., 2009). Regional predictor independent variables with a strong correlation with the two targeted HAI dependent variables were selected for evaluation to develop an efficient Ordinary Least Squares (OLS) regression model. Population density was left out of the OLS regression due to the low variance and because overcrowding was a reasonable proxy to compare to MRSA incidence rates (Immergluck et al., 2019; See et al., 2017). It also had a higher correlation and level of significance to MRSA than density. This effort was also made to increase the U.S. state-based sample size from n=30 to n=32. FIG. 13 shows Correlation of MRSA and Density. FIG. 15 shows correlation of MRSA and Crowding.

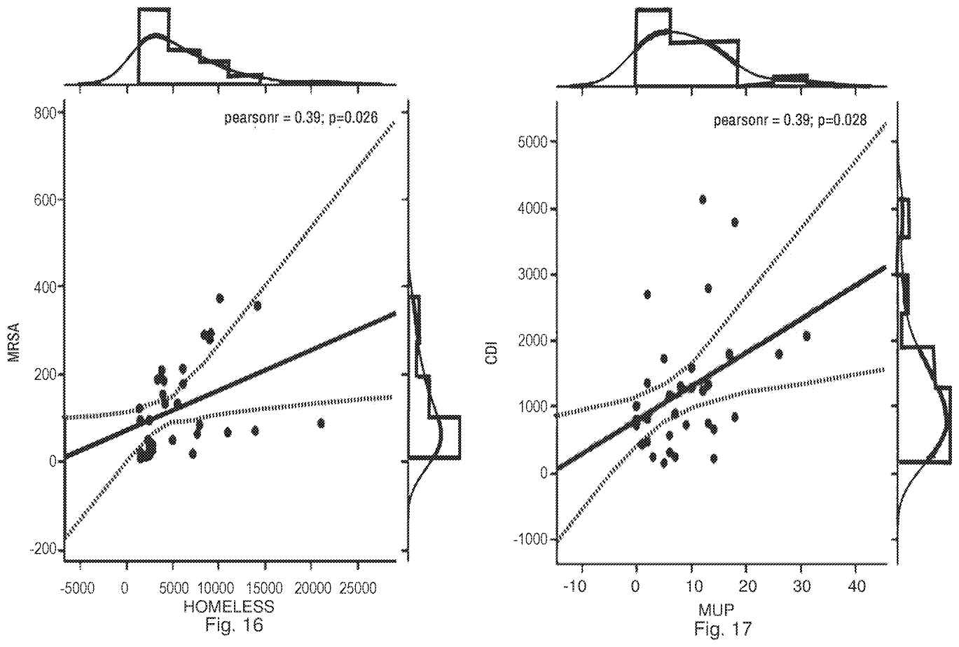

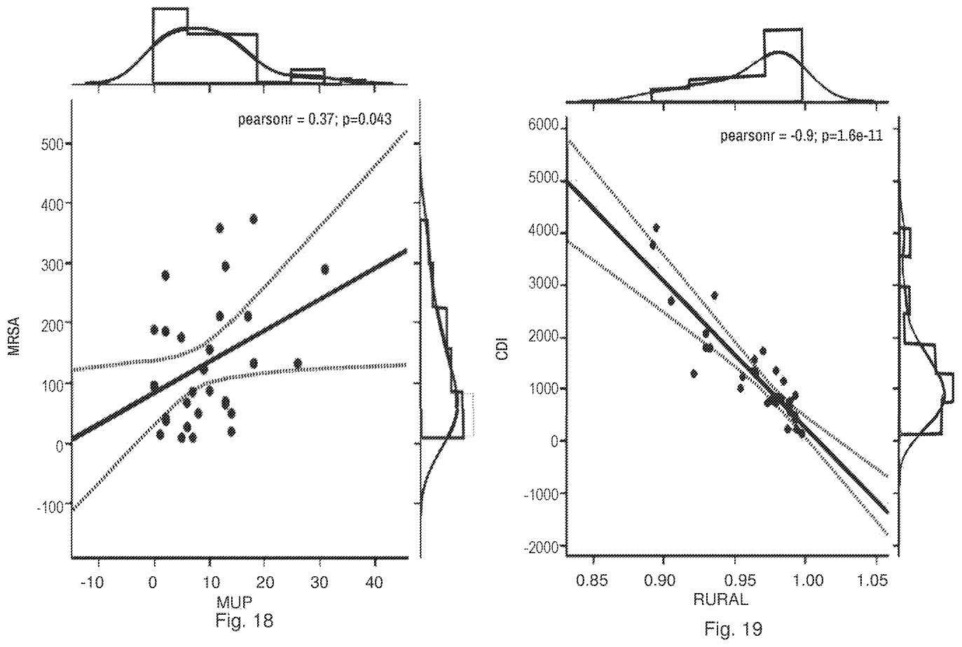

Homelessness, even though the correlation was somewhat weak, was left in as a variable to be regressed against the HAI targets. This choice was made because Homelessness did meet an appropriate alpha of p<0.05 and because there was no other independent variable representation that could serve as a reasonable proxy for this factor. Additionally, the same rationale drove the decision to leave in “Medically Underserved Populations” (MUP). Medically Underserved Areas (MUA) although somewhat related to MUP, as previously explained, is the accrual of area (geographies) rather than populations (people) and therefore not a direct substitute for this variable. The relationship between rural area designation and CDI HAI in the Pearson's r indicated a different relationship than Hypothesis 4a. indicated. The nut hypothesis was accepted. However, the variable was also included in the OLS due to the strong inverse relationship between Rural status and CDI incidence. FIG. 16 shows correlation of MRSA and Homelessness. FIG. 17 shows correlation of MRSA and MUP. FIG. 18 shows correlation of CDI and MUP. FIG. 19 shows correlation of CDI and Rural.

The updated variable relationships and Risk Event likelihood levels are outlined in the post-feature selection Table 7. The associated correlation strength and significance level between HAI target and geographic and demographic predictor variables are indicated in this next iteration of the Risk Analysis matrix.

TABLE 7
Risk Event Evaluation Post-Feature
Selection Likelihood Assignments

| | Risk Likelihood | |
|---|---|---|
| (IF/THEN) | CDI | MRSA |
| Population | Very High/p < .001 | Very High/p < .001 |
| >65 Years Old | Very High/p < .001 | |
| Homeless | | Very High/p < .001 |
| Density | | Somewhat High/p < .05 |
| Crowding | | Very High/p < .001 |
| MU_HSA | Very High/p < .001 | Very High/p < .001 |
| MUP | Somewhat High/p < .05 | Somewhat High/p < .05 |
| Rural | Very High/p < .001 | |
| Urban | | Very High/p < .001 |
| U.S. Geography | Categorical | Categorical |
| DII | | Very High/p < .001 |
| MRSA | Very High/p < .001 | |

* Density variable removed due to assumption “Crowding” is adequate proxy with higher significance and to increase “n.”

The following states that remained in the sample for analysis: Maine; New Hampshire; Pennsylvania; Indiana; Iowa; Kansas; Michigan; Minnesota; Missouri; Nebraska; Ohio; Wisconsin; Alabama; Arkansas; Kentucky; Louisiana; Mississippi; North Carolina; Oklahoma; South Carolina; Tennessee; Virginia; West Virginia; Colorado; Hawaii; Idaho; Montana; Nevada; New Mexico; Oregon; Utah; and Washington. Although smaller in number than the original population, except for the Northeastern region the remaining three regions are relatively equal in amount to one another.

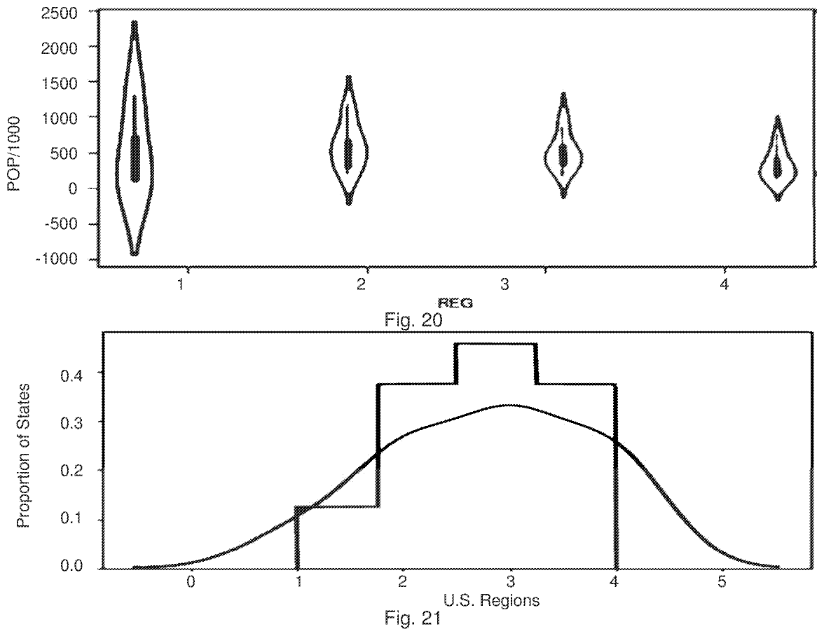

The Northeast Region has the fewest states, but has the biggest proportion of the population, as illustrated by the violin plot in FIG. 20. Trimming of states improved the normality distribution of all four regions as depicted in the distribution graph of FIG. 21.

The sample size and a description of the central tendency for the sample used in the OLS are as follows: U.S. State Population Observations for OLS: n=32; x̅=484.40; M=429.85; sd=307.63. The regional population groupings of Northeast (1), Midwest (2), South (3), and West (4) were also included as dummy variable predictors in the OLS regression. The purpose of adding these regional variables was to evaluate the possibility of determining whether a certain geographic area is associated with specific antibiotic-resistant bacterial HAI incidents. A Sequential Backward Selection (SBS) method using the predictor value of any p>0.05 as a baseline was used to reduce independent variables and improve model fit. The results of the OLS regression using CDI as a dependent variable is shown in Table 8.

TABLE 8
OLS regression results using CDI HAI Incidents as a dependent variable
OLS Regression Results for CDI

| | | | |
|---|---|---|---|
| Dep. Variable: | Y | R-squared: | 0.984 |
| Model: | OLS | Adj. R-squared: | 0.981 |
| Method: | Least Squares | F-statistic: | 313.9 |
| Date: | Mon, 10 Jun. 2019 | Prob (F-statistic): | 2.26E−22 |
| Time: | 17:30:36 | Log-Likelihood: | −199.33 |
| No. Observations: | 32 | AIC: | 410.7 |
| Df Residuals: | 26 | BIC: | 419.5 |
| Df Model: | 5 | | |
| Covariance Type: | Nonrobust | | |

| | Coef | std err | t | P > \|t\| | [0.025 | 0.975] |
|---|---|---|---|---|---|---|
| const | −289.876 | 64.298 | −4.508 | 0.000 | −422.043 | −157.709 |
| SE Region | 250.125 | 99.199 | 2.521 | 0.018 | 46.218 | 454.032 |
| WE Region | 271.663 | 77.945 | 3.485 | 0.002 | 111.445 | 431.880 |
| NE Region | 204.583 | 84.244 | 2.428 | 0.022 | 31.417 | 377.749 |
| Population >65 | 0.001 | 0.000 | 9.671 | 0.000 | 0.001 | 0.001 |
| MRSA | 3.415 | 0.686 | 4.980 | 0.000 | 2.005 | 4.824 |

| | | | |
|---|---|---|---|
| Omnibus: | 2.036 | Durbin-Watson: | 2.292 |
| Prob (Omnibus): | 0.361 | Jarque-Bera (JB): | 1.863 |
| Skew: | −0.535 | Prob (JB): | 0.394 |
| Kurtosis: | 2.497 | Cond. No. | 5.19E+06 |

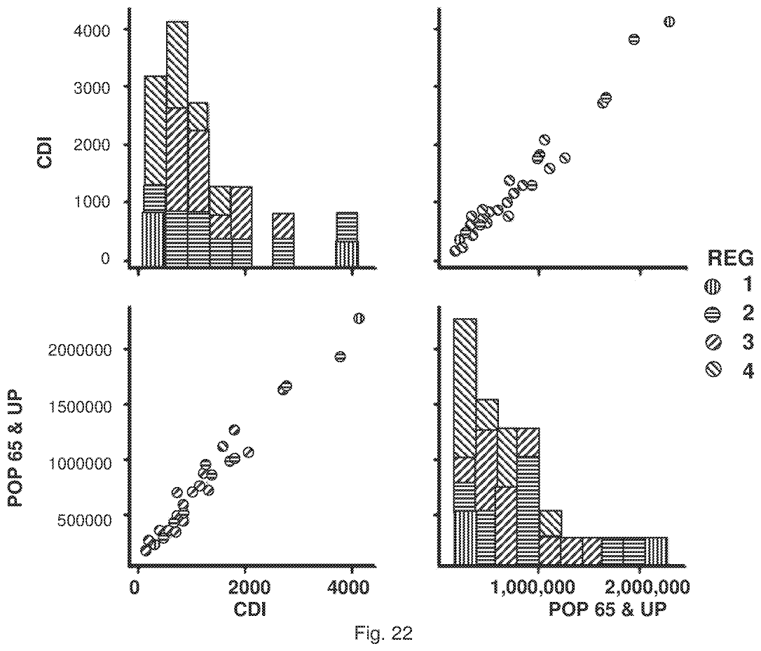

The independent variables selected for this OLS regression model explained ninety-eight percent (R²=0.984) of the variance in the dependent variable prediction. The independent variables that indicated the strongest prediction for CDI HAI were as follows: Southeastern Region: (β=250.125; p<0.01); Western Region: (β=271.663; p<0.001); Northeastern Region: (β=271.663; p<0.05); Population over 65 Years old: (β=0.001); MRSA: (β=3.415; p<0.001). These results suggest that CD HAI incident rates have a significant relationship with certain geographic regions of the U.S. as well as with areas comprised of older populations. Additionally, this analysis implies that the level MRSA HAI is considered as a viable predictor for CU HAI. FIG. 22 shows Pairwise regional comparison graph of Population amount over 65 years and CDI. The same SBS and p>0.05 baseline elimination method were used to reduce predictor variables using nationally reported MRSA HAI incident amounts as a target variable. The results of this OLS Regression model is delineated in Table 9.

TABLE 9
OLS regression results using MRSA HAI Incidents as a dependent variable
OLS Regression Results for MRSA

| | | | |
|---|---|---|---|
| Dep. Variable: | Y | R-squared: | 0.943 |
| Model: | OLS | Adj. R-squared: | 0.937 |
| Method: | Least Squares | F-statistic: | 155.3 |
| Date: | Mon, 10 Jun. 2019 | Prob (F-statistic): | 1.49E−17 |
| Time: | 18:07:10 | Log-Likelihood: | −148.02 |
| No. Observations: | 32 | AIC: | 304 |
| Df Residuals: | 28 | BIC: | 309.9 |
| Df Model: | 3 | | |
| Covariance Type: | Nonrobust | | |

| | Coef | std err | T | P > \|t\| | [0.025 | 0.975] |
|---|---|---|---|---|---|---|
| const | −8.101 | 10.185 | −0.795 | 0.433 | −28.965 | 12.763 |
| MW Region | 73.141 | 9.848 | 7.427 | 0.000 | 52.968 | 93.314 |
| Population | −0.130 | 0.063 | −2.048 | 0.050 | −0.259 | 0.000 |
| CDI | 0.136 | 0.020 | 6.731 | 0.000 | 0.095 | 0.178 |

| | | | |
|---|---|---|---|
| Omnibus: | 0.837 | Durbin-Watson: | 2.388 |
| Prob (Omnibus): | 0.658 | Jarque-Bera (JB): | 0.815 |
| Skew: | 0.168 | Prob (JB): | 0.665 |
| Kurtosis: | 2.294 | Cond. No. | 4.06E+03 |

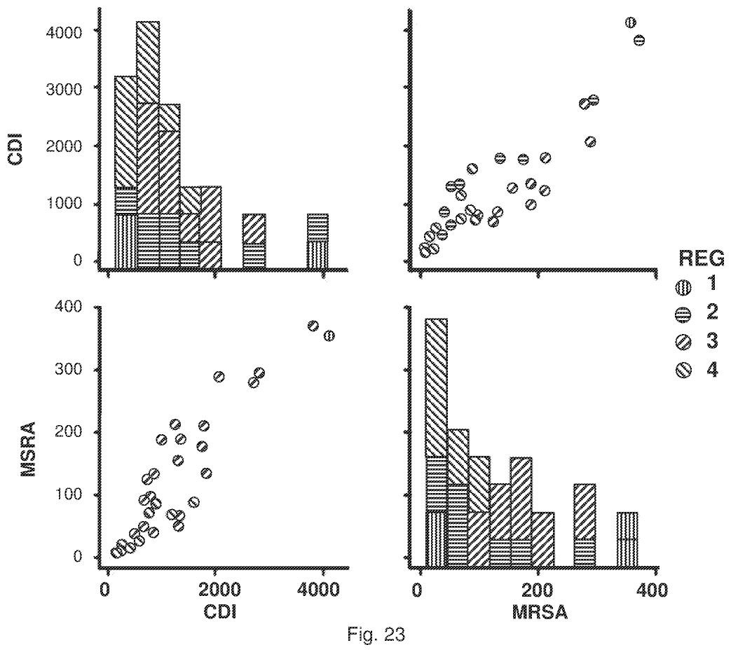

The independent variables selected for this OLS regression model explained approximately ninety-four percent (R²=0.943) of the variance in the dependent variable prediction. The only predictor variables that showed a positive significance level in prediction for MRSA in this model were: Midwestern Region: (β=73.141; p<0.001); CDI: (β=0.136; p<0.001). The results of this analysis suggest that U.S. health systems boated in the Midwestern part of the U.S. and those with higher rates of CDI might use these as indicators of higher levels of risk for MRSA HAI. Interestingly, Population appears to have a slightly inverse relationship with MRSA HAI (β=0.130; p<0.05). Notably, regional influence appears to be a significant predictor for both CDI and MRSA. The pairwise graph below provides a visualization of these regional outcomes. Based on this analysis; it is not clear what specific factors may be driving these results. However, U.S. regionality did appear to be related to both CDI and MRSA HAI incidence rates. FIG. 23 shows Pairwise regional comparison graph of CDI and MRSA

The results of the OLS regression were used to reject or accept the null hypotheses of each of the five Risk Analysis research questions. The results are tabulated in Table 10.

TABLE 10
OLS Relational CDI and MRSA HAI Environmental and Demographic Factors

| | | | |
|---|---|---|---|
| Risk Analysis Research Hypotheses | OLS | R² | Alpha |

Baseline

| | | | |
|---|---|---|---|
| Higher numbers for the regional population are associated with higher | N | 0.981 | NA |
| numbers of CDI incidences | | | |
| Higher numbers for the regional population are associated with higher | N | 0.937 | p < .05 * |
| numbers of MRSA incidences | | | |

Question 1: What is the relationship between regional population compositional factors and the risk for AMR
hospital-onset infectivity?

| | | | |
|---|---|---|---|
| Hypothesis 1a: U.S. regions with a larger proportion of their populations over | Y | 0.981 | p < .001 |
| the age of 65 have an increased risk for incidents of hospital-onset CDI. | | | |
| Hypothesis 1b: U.S. regions with higher populations of homeless persons | N | 0.937 | NA |
| have an increased risk for hospital-onset MRSA. | | | |
| Hypothesis 1c. i: U.S. regions with higher populations densities have an | N | 0.937 | NA |
| increased risk for hospital-onset MRSA. | | | |
| Hypothesis 1c. ii: U.S. regions with higher populations of housing over- | N | 0.937 | NA |
| crowding have an increased risk for hospital-onset MRSA. | | | |

Question 2: How does healthcare accessibility appear to effect and the risk
for AMR hospital-onset infectivity?

| | | | |
|---|---|---|---|
| Hypothesis 2a. i: U.S. regions with more Medically Underserved Areas | N | 0.981 | NA |
| (MUA) have an increased risk for incidents of hospital-onset CDI. | | | |
| Hypothesis 2a. ii: U.S. regions with more Medically Underserved Populations | N | 0.981 | NA |
| (MUP) have an increased risk for incidents of hospital-onset CDI. | | | |
| Hypothesis 2b. i: U.S. regions with more Medically Underserved Areas | N | 0.937 | NA |
| (MUA) have an increased risk for hospital-onset MRSA. | | | |
| Hypothesis 2b. ii: U.S. regions with more Medically Underserved Populations | N | 0.937 | NA |
| (MUP) have an increased risk for incidents of hospital-onset MRSA. | | | |

Question 3: How does the rural or urban status of health systems patient
catchment area affect the risk for AMR hospital-onset infectivity?

| | | | |
|---|---|---|---|
| Hypothesis 3a: U.S. regions with more “rural status” defined areas | N | 0.981 | NA |
| have an increased risk for incidents of hospital-onset CDI. | | | |
| Hypothesis 3b: U.S. regions with more “urban status” defined areas | N | 0.937 | NA |
| have an increased risk for incidents of hospital-onset MRSA | | | |

Question 4: What is the relationship between U.S. geographic region and the
risk for AMR hospital-onset infeCTivity?

| | | | |
|---|---|---|---|
| Hypothesis 4a: There is a relationship between U.S. regional geographic | Y | 0.981 | p < .01; |
| location and incidents of hospital-onset CDI. | | | p < .05** |
| Hypothesis 4b: There is a relationship between U.S. regional geographic | Y | 0.937 | p < 001*** |
| location and incidents of hospital-onset MRSA | | | |

Question 5: What is the relationship between the co-occurrence of hospital-
onset C. diff. and hospital-onset MRSA in acute care hospitals in U.S.
regions?

| | | | |
|---|---|---|---|
| Hypothesis 5a: The incident rates of hospital-onset MRSA are related to | Y | 0.981 | p < .001 |
| the incident rates of hospital-onset CDI. | | | |
| Hypothesis 5b: The incident rates of hospital-onset CDI are related to the | Y | 0.937 | p < .001 |
| incident rates of hospital-onset MRSA | | | |

The results of the OLS analysis avowed for a final reframing of the hypotheses statements as fuzzy IF-THEN statements.

TABLE 11
Hypotheses Fuzzy Membership Rules
after OLS Regression data scaling

| | |
|---|---|
| 1. | IF region Population numbers are large THEN CDI and MRSA Risk |
| | Prevention cannot be associated based on the data analysis. |
| 2. | IF region Population numbers above 65 years old are great, THEN |
| | CDI Likelihood of Exposure is Very High. |
| 3. | IF region Homeless populations are large THEN MRSA Risk |
| | Prevention cannot be associated based on the data analysis. |
| 4. | IF region area population Density proportion is Low, THEN MRSA |
| | Risk Prevention cannot be associated based on the data analysis. |
| 5. | IF region area household Crowding proportion is Low THEN MRSA |
| | Risk Prevention cannot be associated based on the data analysis. |
| 6. | IF region MUA amount is Low THEN, CDI and MRSA Risk |
| | Prevention cannot be associated based on the data analysis. |
| 7. | IF region MUP amount is Low, THEN, CDI and MRSA Risk |
| | Prevention cannot be associated based on the data analysis. |
| 8. | IF region Rural designated area is large THEN CDI Risk Prevention |
| | cannot be associated based on data analysis. |
| 9. | IF region Urban designated area is large, THEN MRSA Likelihood |
| | of Exposure is Very High. |
| 10. | IF regional Geography is in a certain location in the continental U.S. |
| | THEN CDI Likelihood of Exposure is High, and MRSA Likelihood |
| | of Exposure is Very High. |
| 11. | IF regional MRSA incidence rates are Low, THEN CDI Likelihood |
| | of Exposure is Very High. |
| 12. | IF regional CDI incidence rates are Low, THEN MRSA. Likelihood |
| | of Exposure is Very High. |

This effort allowed for the manifestation of the geographic and demographic factors most closely associated with CDI and MRSA HAI to become salient. The specificity of this information was notable for several reasons. One reason included obtaining validated metrics to govern the firing strengths of Risk Analysis variables within Fuzzy Risk Prevention membership categories and determining the specific areas of HAI risk so that they were aligned with certain areas of resilience interventions that were assumed tenable. Furthermore, the OLS method has proven to be useful in selecting the essential Fuzzy Rules based on their contributions of variance and significance to the analysis output. (Yen & Wang, 1999). The associated significance level between HAI target and geographic and demographic predictor variables are indicated in the final iteration of the Risk Analysis matrix.

TABLE 12
Risk Event Evaluation Post-OLS Regression
Selection Likelihood Assignments

| | Risk Likelihood | |
|---|---|---|
| (IF/THEN) | CDI | MRSA |
| Population | N/A/p < .05 | N/A/p < .05 |
| >65 Years Old | Very High/p < .001 | |
| Homeless | | N/A/p < .05 |
| Density | | N/A/p < .05 |
| Crowding | | N/A/p < .05 |
| MU_HSA | N/A/p < .05 | N/A/p < .05 |
| MUP | N/A/p < .05 | N/A/p < .05 |
| Rural | N/A/p < .05 | |
| Urban | | N/A/p < .05 |
| U.S. Geography | High/p < 01 | Very High/p < .001 |
| DII | | Very High/p < .001 |
| MRSA | Very High/p < .001 | |

The following sections discuss how this probabilistic environmental and population hazard data evaluation (i.e., Risk Analysis) guided the possibilistic adaptive response intervention appraisal (i.e., Resilience Assessment). Additionally, a review of how both processes can be integrated into a Fuzzy Inference System is discussed. The present technology provides a comprehensive Resilience Inference approach that offers improved insight into CDI, and MRSA HAI prevention performance safety when Risk, Resilience, and associated Performance Safety outcomes are considered.

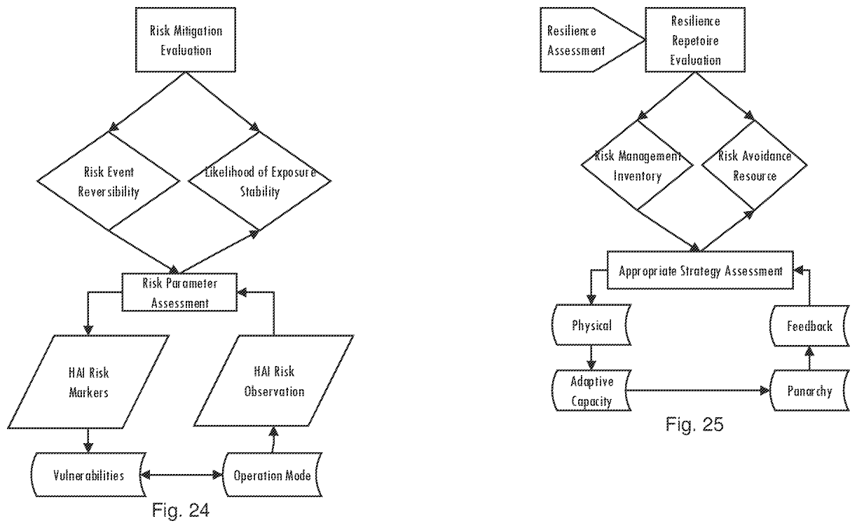

FIG. 24 shows HAI Risk Mitigation Evaluation Process. Risk Event Evaluation categories (e.g., alone) may be used to build a fuzzy rule basis and incorporated with Resilience fuzzy membership values into a Fuzzy Inference System (FIS) that elicits crisp quantitative safety outcome variables. However, returning to the Resilience Assessment Model process categories as an operationalization guide indicates that Risk Mitigation circumstances must be considered as part of this progression. Conditions for hazard prevention are different from adaptive unknown risk scenario planning, but are relevant to understanding resilient response (Hollnagel et al., 2006).

Table 13 describes the associated metrics assigned to the three phases of Risk Analysis.

TABLE 13
Risk Analysis: Event Likelihood, Mitigation
Potential, Prevention Level Metrics

| | | | Risk Mitigation | | | |
|---|---|---|---|---|---|---|
| Risk Event Evaluation | | | Evaluation | | | |
| Hazard | Likelihood of | | Stability | Reversibility | | |
| Event | exposure (L) | | (S) | (R) | Risk Prevention | |
| CDI | Somewhat | 0.05 | 100 | 100 | Somewhat | 0.60-1.0 |
| | High | | | | Low | |
| MRSA | High | 0.01 | 50 | 50 | Low | 0.30-.70 |
| | Very High | 0.001 | 25 | 25 | Very Low | 0-.40 |

Predictive likelihood metrics for Risk Event Evaluation factors are determined by their significance level resultant from the OLS regression analysis of HAI predictor and target variables. Likelihood metrics for Risk Event Evaluation serve as firing strengths weights in defining the degree of membership in Risk Prevention fuzzy category continuum. The Risk Analysis operationalization framework is also contingent upon defining system mode of operation Condition Stability and Reversibility. It is presumed the same membership scale metrics that are used for Risk Prevention fuzzy membership levels is used for defining capacity levels for these two areas to simplify the analysis.

TABLE 14
Risk Analysis Mode of Operation Condition
Stability and Reversibility Levels

| Condition Stability (S) | | Condition Reversibility (R) | |
|---|---|---|---|
| High Stability | 60-100 | Easily Reversible | 60-100 |
| Moderate Stability | 30-60 | Somewhat Reversible | 30-60 |
| Low Stability | 0-40 | Difficult to Reverse | 0-40 |

An actual model of operation capacity levels may be set by specific clinical SME within the health systems undertaking HAI Resilience Inference, with distinct area dependent factors (e.g., cities, locales, and neighborhoods). Predictor variables related to patient demography proportion and other community-specific geographically dependent variables diverge depending on where in the U.S. defined region, even when the Resilience inference was taking place. This may be accounted for using known techniques. Arbitrary levels for Risk Mitigation Condition Stability and Condition Reversibility are used for illustrating this phase of HAI Risk Analysis.

The Risk Prevention Fuzzy Membership levels are defined by the TEN and TrFN illustrated in FIG. 4. The level of HAI-related probability is compared with an associated possibility metric for prevention or Risk Mitigation Capacity, to determine the Risk Prevention Fuzzy Membership levels for certain types of HAI Risk Events, using a weighted average approach. The significance level of the likelihood of the risk event (L) serves as an associated multiplier for the averaging of risk mitigation stability (S) and reversibility (R) conditions ranking assigned by health system SME. The presumed stability and reversibility rankings of each of the significant CII and HAI risk event factors are outlined in Table 15.

TABLE 15
Presumed Risk Mitigation Mode of Operations Rankings

Risk Mitigation Evaluation

| | Assumed | Assumed |
|---|---|---|
| Risk Event Evaluation (REE) | Stability (S) | Reversibility (R) |
| Age and CDI | 50 | 25 |
| Geography and CDI | 100 | 25 |
| Geography and MRSA | 100 | 25 |
| CDI due to MRSA Incidence | 50 | 100 |
| MRSA due to CDI Incidence | 50 | 100 |

Given the Risk Event weighting and mitigation potential levels, a Risk Prevention level may be calculated based on a compounded level of Risk Event Evaluation, (REE) and Risk Mitigation Evaluation (RME) amounts. The formula for this approach is delineated in Table 16 in relation, to each of the five risk events resulting as significant by the OLS.

TABLE 16
Calculations for HAI Risk Prevention Capacity μF(X) degree of membership

| | | | | | |
|---|---|---|---|---|---|
| | | Risk | | | Percentile |
| | | Mitigation | | Risk | Rank |
| Risk Event | Weighted average | Evaluation | Average | Prevention | REE + |
| Evaluation (REE) | ((S(L) + R(L)/(L + L))/100 | (RME) | (S + R)/N)/100 | Ranking | (REE*RME) |

| | | | |
|---|---|---|---|
| Age and CDI | 0.1255 | 0.375 | 0.173 |
| Geography and CDI | 0.135 | 0.625 | 0.219 |
| Geography and MRSA | 0.126 | 0.625 | 0.205 |
| CDI due to MRSA | 0.5005 | 0.75 | 0.876 |
| Incidence | | | |
| MRSA due to CDI | 0.5005 | 0.75 | 0.876 |
| Incidence | | | |

This computation provides the information needed for Risk Fuzzy Set input level metrics to incorporate into a FIS that provides specificity on HAI prevention and adaptive response expectations.

FIG. 24 shows HAI Resilience Assessment Process. As previously mentioned, the classifications of risk characteristics and the significance of their relationship to specific HAI like CDI and MRSA were necessary for the alignment of viable and practical adaptive response strategies. Based on the Risk Analysis results presented the need for this process step should be more evident because of how this action both assists in substantiating Risk Event Evaluation fuzzy membership categories as wet as helping to define the boundaries of Resilience related fuzzy sets.

TABLE 17
Risk and Resilience Inputs into FIS

| | Risk Prevention | Resilience Potential | |
|---|---|---|---|
| HAI Risk | Membership Level | Membership Level | |
| Event Factor | (FIS-Input 1) | (FIS-Input 2) | |
| Age above | 0.173 | 78% reduction in CDI | .780 |
| 65 and CDI | | (copper-physical) | |
| Geography | 0.219 | 42.7% reduction in | .427 |
| and CDI | | CDI (panarchy) | |
| Geography | 0.205 | 30% reduction in | .300 |
| and MRSA | | MRSA (decolonization- | |
| | | adaptive capacity) | |
| CDI due | 0.876 | 42.7% reduction in | .427 |
| to MRSA | | CDI (panarchy) | |
| Incidence | | | |
| MRSA due to | 0.876 | 31% reduction in | .310 |
| CDI Incidence | | MRSA (feedback) | |

The interventions indicated in Table 17, although specific to HAI type, are arbitrary and for model demonstration. For example, a “Panarchy-based” strategy is substituted for the microbial resistant copper physical intervention for Age above 65 and CDI.

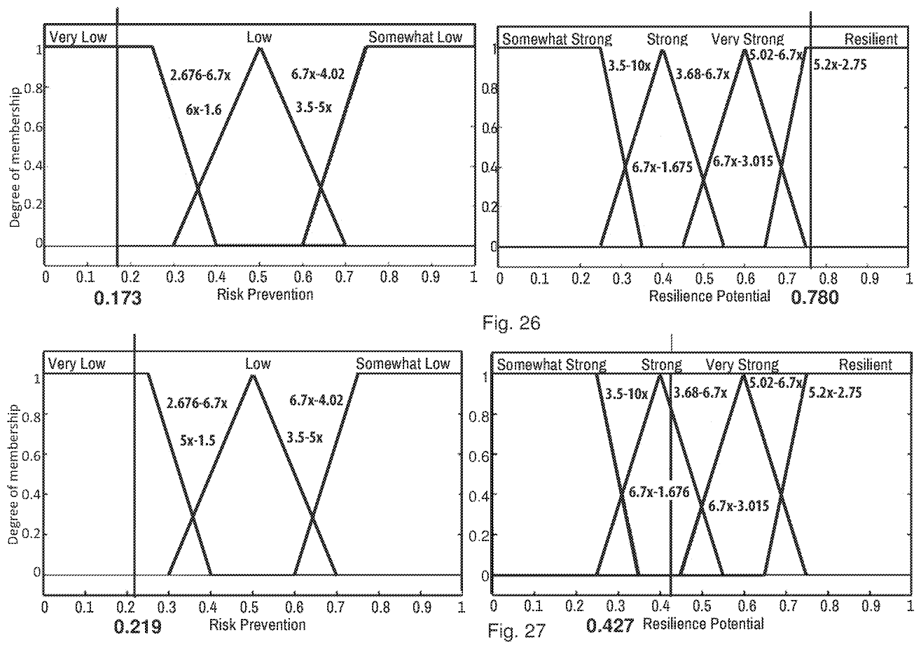

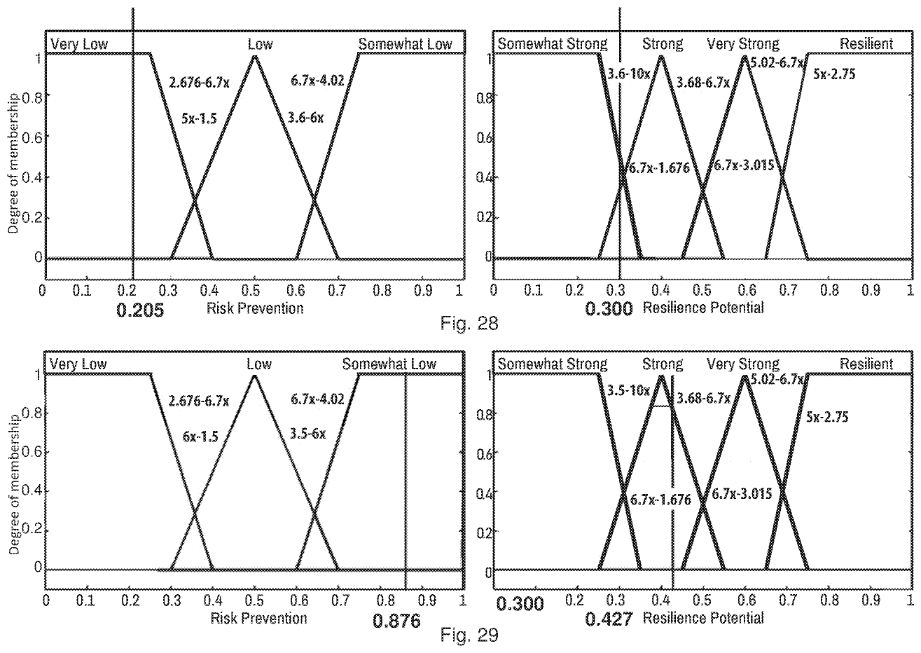

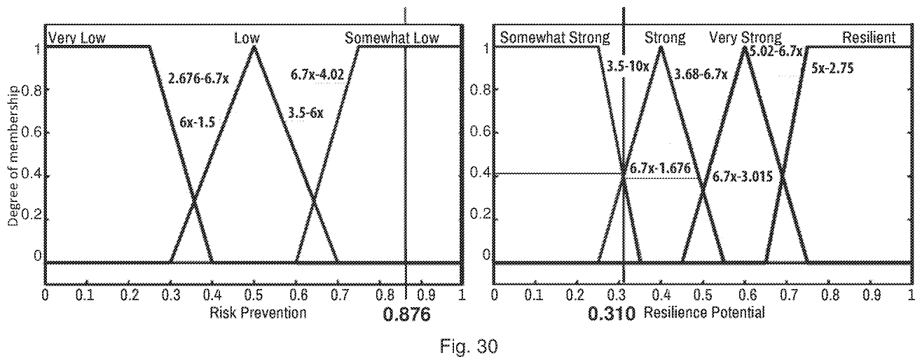

Based on the input risk and resilience fuzzy membership levels, the intersection of the two truncated disjoint membership functions fats within the fuzzy membership continuums of Risk Prevention and Resilience Potential. FIG. 26 shows Risk μF(X): Age Above 65 and CDI; Resilience μF (X): Copper in Healthcare EOC Finishes: FIG. 27 shows Risk μF(X): Geographical Region and CDI; Resilience μF (X): Panarchy of operational prevention. FIG. 28 shows Risk μF(X: Geographical Region and MRSA; Resilience μF (X): Decolonization regimen post-discharge. FIG. 29 shows Risk μ_{F} (X): MRSA and CDI; Resilience μF (X): Panarchy of operational prevention. FIG. 30 shows Risk μ_{F} (X): CDI and MRSA; Resilience μF (X): Clinical feedback standard operating procedure. These combined memberships present the opportunity to be evaluated together using a centroid analysis to establish a crisp output that allows for an inference of associated HAI prevention performance safety consequents.

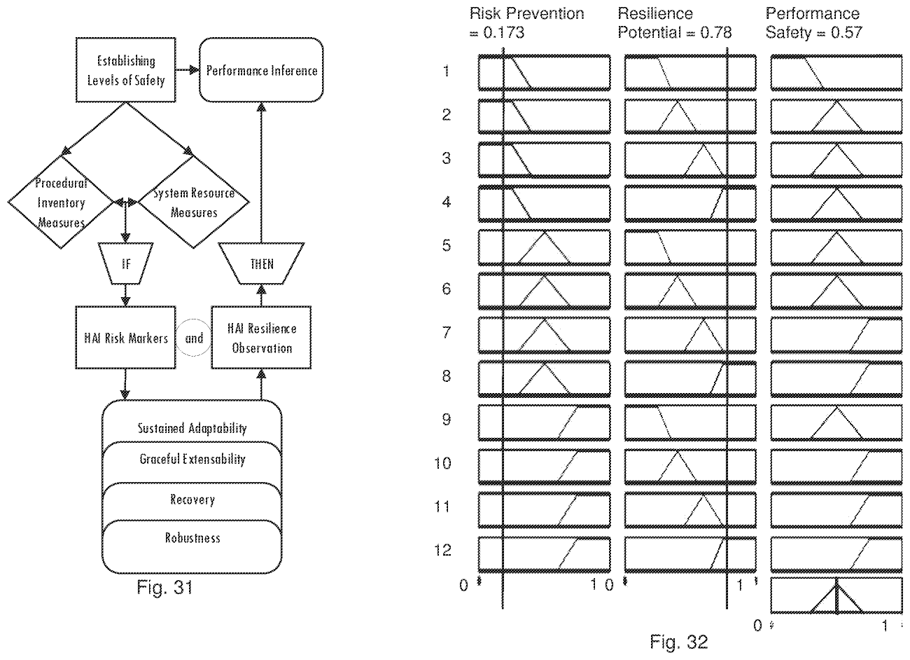

FIG. 31 shows the HAI Performance Safety Inference Process. The information generated in the Risk Analysis, and selection of specific HAI responsive case study derived Resilience Assessment data strategies and associated rule basis may be used in a FIS structure, to make inferences regarding the different combined attributes of a risk event and risk-adjusted resilience on infection control safety outcomes. For examining the performance safety outcomes from this FIS, the risk factors defined as significant by OLS continue to be used. Each of these now has two associated risk and resilience fuzzy membership assignments that can be used as inputs in the FIS.

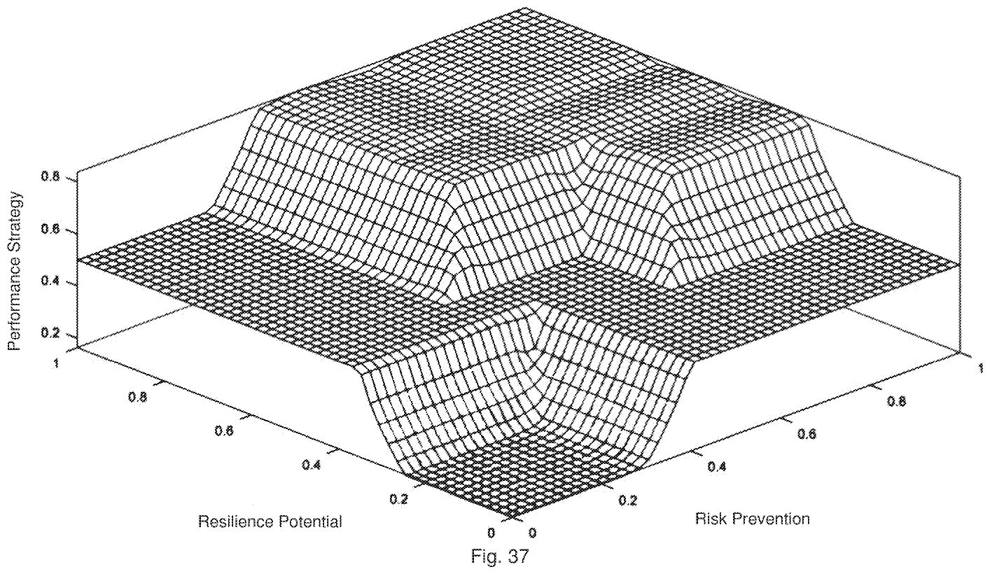

Based on the inputs of the Risk and Resilience examples, the results join these two memberships. Specifically, a strong or higher resilience achievement membership level improves HAI adaption performance safety even if the potential to prevent HAI risk were very low. Inputting the “Fuzzy Rule Basis for Resilience Inference and HAI Performance Safety”, permits visualization of the juncture of fuzzy TFN and TrFN of risk prevention and resilience potential as it relates to Performance Safety. FIG. 37 shows Surface Map for Risk Prevention Resilience Potential and Performance Safety μ_{F}(X).

CDI and MRSA HAI specific fuzzy membership risk prevention levels can now be input along with case-study derived intervention strategies deemed as most relevant to CDI and MRSA HAI, as resilience potential fuzzy membership levels. The fuzzy membership inputs, when evaluated together using a centroid analysis to establish a crisp output in the context of the fuzzy rule basis, offers the following crisp outputs as it relates to HAI control performance safety.

TABLE 18
HAI Resilience Inference FIS Performance Safety Results

| | Associated Risk | | | | | |
|---|---|---|---|---|---|---|
| Risk Factor and | Prevention Ranking | | HAI Specific Resilience | Performance Safety Inference | | |
| HAI Event | Input | | Potential Input | Output | | |
| Age and CDI | 0.173 | Very Low | 78% (0.780) | Resilient | 0.50 | Safe |
| Geographical | 0.219 | Very Low | 42.7% (0.427) reduction | Strong | 0.50 | Safe |
| Region and CDI | | | in CDI (panarchy) | | | |
| Geographical | 0.205 | Very Low | 30% (0.300) reduction | Somewhat | 0.297 | Somewhat |
| Region and MRSA | | | in MRSA (decolonization) | Strong/Strong | | safe |
| CDI due to | 0.876 | Somewhat Low | 42.7% (0.427) reduction | Strong | 0.831 | Very Safe |
| MRSA Incidence | | | in CDI (panarchy) | | | |
| MRSA due to | 0.876 | Somewhat Low | 31% (0.310) reduction | Somewhat | 0.672 | Safe/Very |
| CDI Incidence | | | in MRSA (feedback) | Strong/Strong | | Safe |

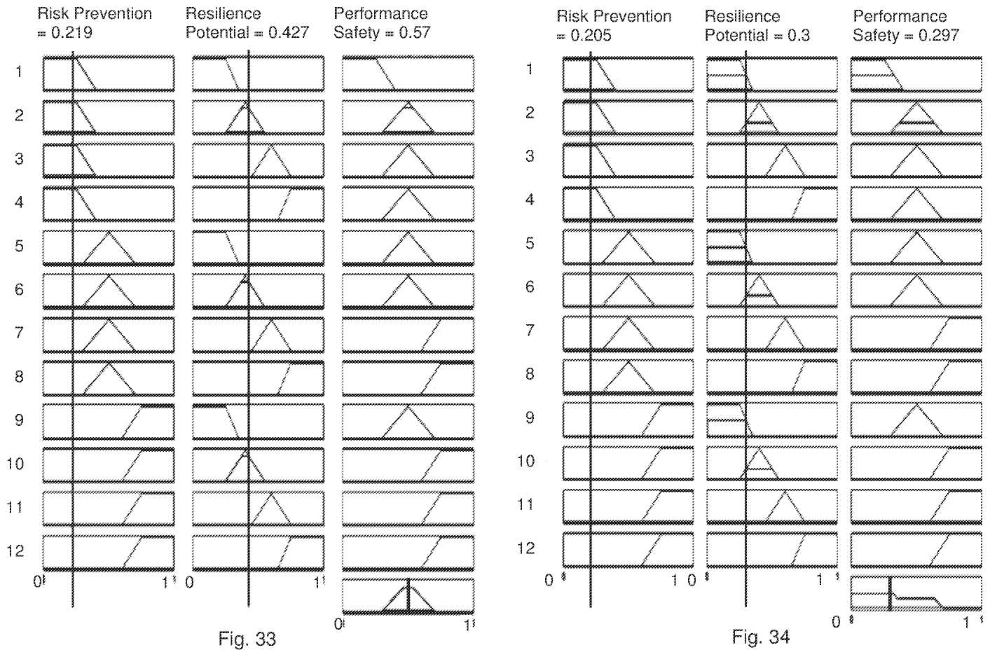

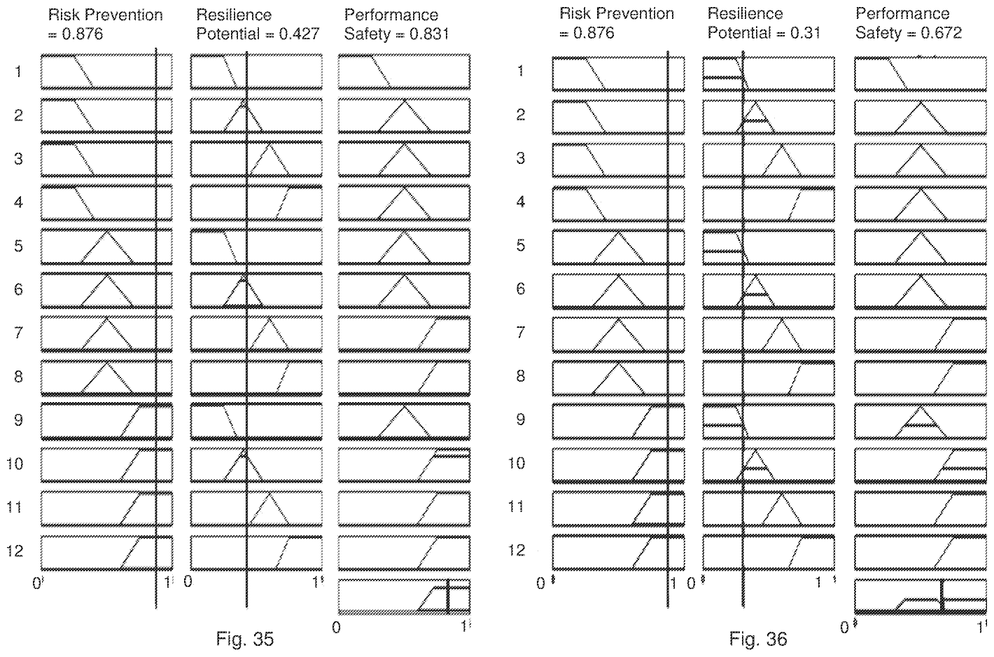

The visualizations for the above referenced FIS Performance Safety results outlined in Table 18 are illustrated in FIGS. 32-36. FIG. 32 shows Performance Safety FIS Outcome for Risk μF (X); Age Above 65 and CDI; Resilience μ_{F}(X): Copper in Healthcare EOC Finishes FIG. 33 shows Performance Safety FIS Outcome for Risk μF (X): Geographical Region and CDI; Resilience μ_{F}(X): Panarchy of operational prevention. FIG. 34 shows Performance Safety FIS Outcome for Risk μ_{F}(X): Geographical Region and MRSA; Resilience μF (X): Decolonization regimen post discharge. FIG. 35 shows Performance Safety FIS Outcome for Risk μF (X): MRSA and CDI: Resilience μ_{F}(X): Panarchy of operational prevention. FIG. 36 shows Performance Safety FIS Outcome for Risk μF (X): CDI and MRSA; Resilience μ_{F}(X): Clinical feedback standard operating procedure.

This analysis is adequate to test the assumption that a strong or higher resilience achievement membership level could improve HAI adaption performance safety regardless of its risk prevention level. The continuum of resilience prevention spans the range of the lower levels of “Strong” (e.g., post-discharge decolonization regimen at 30% MRSA HAI reduction) to “Resilient” (e.g., copper finishes installed in acute care inpatient settings at 78% CDI HAI reduction). The number of tested HAI Resilience Inference observations is small, which is to a degree contingent upon the data available for testing. However, the distribution of these outcomes is generally parametric. A t-Test that compares the associated likelihood levels of Risk Event Evaluation with the fuzzy inferred Performance Safety likelihood levels considering resilience strategy integration suggests the difference between these outcomes is significant,

TABLE 19
t-Test comparison of Risk Event and HAI Performance
Safety Likelihood t-Test Paired Two Sample for Means

| | Risk Likelihood | Safety Likelihood |
|---|---|---|
| Mean | 0.278 | 0.56 |
| Variance | 0.041 | 0.0405885 |
| Observations | 5.000 | 5 |
| Pearson Correlation | 0.871 | |
| Hypothesized Mean Difference | 0.000 | |
| Df | 4.000 | |
| t Stat | −6.141 | |
| P(T <= t) one-tail | 0.002 | |
| t Critical one-tail | 2.132 | |
| P(T <= t) two-tail | 0.004 | |
| t Critical two-tail | 2.776 | |

These results validate the assumption that integrating even a comparatively low cost and easy to implement resilience improvement strategy to augment healthcare delivery infrastructure, may offer a meaningful impact on improving HAI control Performance Safety outcomes.

TABLE 20
Extending Risk Analysis Hypotheses
Rule Basis through FIS output

| | |
|---|---|
| 1 | IF CDI associated Risk Prevention is Very Low because |
| | healthcare region's population numbers above 65 years old are |
| | large AND Health Systems Infrastructure Resilience is Resilient, |
| | THEN C. diff. HAI Prevention Performance is Safe. |
| 2. | IF CDI associated Risk Prevention is Very Low because the |
| | regional geography of the patient catchment area is in the |
| | southeastern, western, or northeastern part of the U.S. |
| | AND Health Systems Infrastructure Resilience is Strong |
| | THEN C. diff., HAI Prevention Performance is Safe. |
| 3 | IF MRSA associated Risk Prevention is Very Low because the |
| | regional geography of the patient catchment area is in the |
| | midwestern part of the U.S. AND Health Systems |
| | Infrastructure Resilience is Somewhat Strong to Strong |
| | THEN C. diff. HAI Prevention Performance is Somewhat Safe. |
| 4. | IF CDI associated Risk Prevention is Somewhat Low because |
| | MRSA HAI incident rates are high AND Health Systems |
| | Infrastructure Resilience is Strong, THEN C. diff. HAI |
| | Prevention Performance is Very Safe. |
| 5. | IF MRSA associated Risk Prevention is Somewhat Low |
| | because of C. diff. HAI incident rates are high, AND |
| | Health Systems Infrastructure Resilience is Somewhat |
| | Strong to Strong THEN MRSA HAI Prevention Performance |
| | is Safe to Very Safe. |

There are several limitations to the Risk Analysis portion of this evaluation that are noteworthy. The approach selected for identifying regional U.S. risk factors associated with HAI caused by antimicrobial-resistant bacteria required an assumption of normally distributed data. However, even with the removal of state-based outliers, it is evident that none of the distributions of variables selected was perfectly normally distributed. This analysis only viewed incidences of C. diff. and MRSA infectivity in acute care settings.

Individual states were trimmed from this analysis, such as California, Florida, New York, and Texas, due to the presence of both population and environmental based outliers. The removal of these states improved normality of the regional data distribution and thus potentially increased the potential of model outcomes assumption generalizability. However, all four of these states based on the most current 2017 CDC SIR data rank the highest in both CDI and MRSA observed HAI in acute care settings. Therefore, availability of state-specific data with recorded discrete community-based HAI observations across different acute care health systems, rather than the accumulated lump sum HAI data, is beneficial.

Currently, the only real potential for gathering HAI resilience, data is to draw it from discrete peer-reviewed published case studies. These investigations typically are done under specific conditions and with a comparatively much smaller N-value than those found in nationally reported HAI rates. This makes the generalizability between Risk Factor and Resilience Potential data sources particularly challenging. However, using evidence-based data, while not as robust as Supervised Learning data analysis efforts is at present, is one of the only feasible ways of comparing risk and resilience-based outcomes that relate to hospital-onset infections.

The Resilient Systems Inference Model provides a somewhat self-contained interactive system performance forecasting and evaluation framework that healthcare organizations could feasibly apply independently. Health systems are often understandably reticent to expose internal challenges they may be having regarding infection control and HAI incidence to external researchers or consultants that have advanced system analysis expertise. This issue can make it difficult for healthcare organizations to gain meaningful and accurate insight into the potential value, or lack thereof, their infection prevention strategies have on increasing patient safety.

Performing an environmental scan of a hospital's regional catchment area could demonstrate if a significantly high percentage of their patient population was over the age of 65. This could make a compelling case for a facility investing in well tested environmental contact surfaces resilient to Clostridioides difficile pathogen propagation such as antimicrobial copper. Additionally, hospitals based in the midwestern portion of the U.S. might be well advised to implement a low-cost system of clinical feedback related to patients presenting with MRSA in acute care settings in order to improve operational resilience to associated HAI incidences in their inpatient population. Neither of these options requires any substantive additional analysis on hospital administrators' part, but both offer an evidence-based approach to potentially improving resilience potential of acute care HAI reduction strategies.

Arbitrary levels for Risk Mitigation “Condition Stability” and “Condition Reversibility” were used because the actual operation capacity levels for these two factors need to be assigned by specific clinical and administrative SME within specific health systems. As indicated, exogenous system area dependent factors (e.g., cities, locales, and neighborhoods) and potentially endogenous health system unit-based factors (e.g., ICU, Med/Surg, and Labor and Delivery) wad both influence these metrics. If the Resilient Systems Inference Model presented were to be used by an individual health system, Fuzzy Cognitive Mapping is used as a way of improving the validity and accuracy of Risk Mitigation “Condition Stability” and “Condition Reversibility” measures.

Fuzzy Cognitive Mapping (FCM) is a technique that captures the relationships between both human and engineered system elements. FCM graphs structure that provides a human-driven and flexible method for intuitively representing complex relationships between endogenous and exogenous system elements. FCM is constructed of intersecting cognitive concept nodes representing task-related events; the links that connect the nodes are then assigned vague “fuzzy” strengths in the interval range [−1,1], indicating the degree to which one event influences another (Smith and Eloff, 2000). A critical component to realizing effective and safe workflow design, which also avoids situational or systemic error, is to ensure that human cognition is endemic to process development (Sutcliffe, 2006). Additionally, a primary focus of Human Factors safety is how to best design shared cognitive systems so that people working in groups can perform successfully in the diverse circumstances in which they have to function (Woods et al., 2017). Furthermore, the construct of Fuzzy Logic is not only a proper way of capturing the quantitative interpretation of linguistic measures but a useful method for human learning and development in natural and, technology-based environments and should, therefore, be treated by the system designers and engineers as a valuable component for establishing a process or place design requirements (Karwowski et al., 1999).

FCM offers a promising method for approaching an overall understanding of risk mitigation potential based on care delivery workflow within specific environments of care serving unique patient populations. In HAI Risk Mitigation workflow development, an understanding of the cognitive functioning supports and impediments that are placed on both clinical and operational support staff is important for successful care delivery to patients in complex and mutable settings. Cognitively complex situations increase the potential for risk and human error (Reason, 2016). Using FCM for analysis of specific HAI Risk Mitigation potential or barriers within clinical workflow could provide more context-relevant data that aids in better defining specific types of HAI “Condition Stability” and “Condition Reversibility” level metrics.

Although, overcrowding and Homelessness did not register in the OLS as being statistically significant risk indicators of incidences of MRSA. They did register as having a significant relationship with MRSA in the Pearson's r. There also appears to be a visual relationship between the factors of Homelessness and Crowding as it relates to the incidence of MRSA. Some research has linked these environmental factors with increased observed incidences of MRSA (Immergluck et al., 2019; See et al. 2017). Moreover, MRSA has the potential of being classified as an endemic infection in vulnerable populations, an epidemic if occurring with flu and or pneumonia, or a pandemic if occurring with a virulent pathogenic strain of Type A Influenza (MacIntyre & Bui, 2017). These characteristics make it a worthwhile area for improving healthcare resilience in general (Schoch-Spann et al., 2018).

There is growing evidence that there are many environmental conditions that are suspected as being linked to the pervasiveness of pathogens that cause human infectivity. Although “rural” designated land areas were evaluated as part of this analysis, this factor had a significantly inverse relation with both CDI and MRSA. However, most of the land is in the U.S. is designated as rural (Ratcliffe et al., 2016). The terms “rural” and “agricultural” do not necessarily have the same meaning and much of the rurally designated area in the U.S. is not used for the type of farming purposes that exposes humans to the type of waste streams directly associated with Clostridioides difficile infectivity (Brown & Wilson, 2018; Freeman et al, 2010). Geographic information system (GIS) data that indicated specific regions used for high infectivity-risk agricultural waste stream exposure along with the incidence of hospital and community-onset CDI offers a depiction of the relationship between this type of environmental risk factor and HAI. In addition to regional commercialization patterns such as agricultural exposure, there is growing research that links climatic conditions to infectivity prevalence. Research on areas with warmer temperatures have drawn links between these climatic conditions and the prevalence of both CD and MRSA (Sahoo et al. 2017; Naggie, 2010). Additionally, research on climate change has suggested that there is a relationship between warming temperatures and AMR in general (European Society of Clinical Microbiology and Infectious Diseases, 2019; MacFadden et al., 2018).

The effects of warming temperature and HAI prevalence also raises questions about measuring the effects of relevant HAI risk factors in urban environments. Research indicates that urban areas due to socioeconomic factors can struggle with MRSA (See et al. 2017) and there does appear to be a visible relationship in the data used for this study and incidence of MRSA in Urban areas. However, this incidence rate may be related to the prevalence of the “urban heat island effect” that can cause cities on a yearly average to be significantly warmer than non-urban areas, except for those urban areas in biomes with arid and semiarid climates (Imhoff et al., 2010). The effort to delve more deeply into urban conditions, which lead to increased HAI risk factors is another area that is aided through access to GIS specific data and area-specific HAI rate data. More generally, the resilience model may be location-based, and dependent on geographic information system information and/or weather or climactic conditions.

Principal Component Analysis (PCA), Spatial PCA, and other techniques for analysis of location and geographic information, may be employed as part of the initial data analysis and/or preparation of the actions to be applied. Shandoosti (2016), Wang (2017).

Environmentally based resilience interventions, such as using copper for high contact surfaces in patient treatment areas, are often more expensive but could also prove more stable than behavior change because they are fixed improvement interventions. A common way of determining the cost-benefit value of environmental investments is by conducting a Life-cycle cost analysis (LCCA). This process assesses all the costs associated with acquiring, maintaining, disposing, and replacing a building material, fixture, or system. LCCA is especially valuable when design or building project alternatives exceed the initial or “first costs” of items which fulfill the same performance requirements but may not have the same performance features. LCCA allows high-performance, but higher first cost alternative system components to be compared similar lower first cost but weaker performance system elements to select the one that maximizes net saving (Fuller, 2016).

Cost-benefit analysis (CBA) is a way to incorporate LCCA and human costs to assess the net benefit of implementing specific operational improvement initiatives. These often highly detailed and longitudinal analysis when used for complex organizational improvement and capital planning development include not only initial construction and actual procurement costs but also the following (Abernathy, 2012): Project-people costs, which are estimates of associated work hours required by full-time employees or subcontractors required to implement the improvement initiative; Target-people costs refer to the cost of time needed to train people interacting with the new system to use and upkeep it so that it maintains operational effectivity. The estimate also includes any downtime or lost production time due to project implementation; Technical-support costs refer to fees paid to consultants or product providers who needed for quality control, installation, debugging, and other required support for strategy implementation; Resource costs are the cost of materials or equipment required to support or mobilize the improvement initiative; Maintenance costs are annualized expenses required for maintaining the solution after its implementation.

Using CM provides a viable way of evaluating the advantage of integrating specific HAI resilience strategies. As a means of demonstrating the value of CBA applied to HAI resilience strategies using data tom case studies related to cost savings and patient safety accessed for this research can be employed for demonstrating the value of this approach.

There have been several studies that have evaluated the antimicrobial performance of copper as a healthcare environment finish. Measurable CDI HAI reductions have been observed in health systems that have used copper as an experimental contact surface in comparison to patient rooms that did not have this type of surface treatment instated (Sifri et al., 2016; von Dessauer et al., 2016). There have also been studies related to the surplus cost of inpatient care that can be directly attributed to hospital-onset CGI (Zhang et at 2018; McGlone et at 2012). Not only do HAIs impact hospital reimbursement from CMS (CMS, 2018), they also can impact patient perceptions of care quality and experience which may further impact both CMS reimbursement based on patient experience as wet as current and future hospital revenue (Burnett et al, 2010). This scenario presents an opportunity to evaluate how a health system may amortize higher initial costs that may be incurred in the procurement of copper Finishes, Fixtures, and Equipment (FFE) over time through recouped patient care savings. A cost-benefit model for the use of copper fixtures was developed by The University of York, in the U.K. that facilitates the input of first cost pricing for patient room furnishings with copper and “regular” finishes. Some of these furnishings included copper finished: Bedrails; Overbed tray table; Call button; IV Pole. The York cost-benefit model template is based on a 20-bed ICU and predicts payback for investing in antimicrobial copper furnishings for patient treatment areas in less than one year (Taylor et al. 2013).

Using resilient systems inference for estimating hospital-acquired infection prevention infrastructure provides a valuable tool for forecasting performance safety outcomes for infection control strategies based on health system risk capacity and resilience capability. Furthermore, this approach assists in informing health resource planning with valid evidence, and as a result, improve adaptive capacity in medically underserved urban and rural geographic locations where vulnerable patients are most at risk of contracting HAI. Another benefit for using resilient systems inference model is to enable diverse healthcare teams to achieve infection prevention and control standards along with regional health resources better suited to safe, effective, and sustainable care delivery for their own regional patient population needs.

Hospital-onset infections present a significant risk in undermining the health and well-being of many people tying in the U.S. and worldwide. The rise of community-based infections and growing level of antimicrobial resistance due to factors outside the scope of control of health systems exacerbates this risk. Poor quality in preventable hospital-onset infections can create a vicious cycle of increased incidence rates in acute care inpatient environments that can be extremely costly to health systems. A system's performance HAI resilience inference model permits analysis of underexplored regional, environmental, and social risk infectivity factors. Concurrent adaptive response efficacy of both available and needed health resources are invaluable to infection control planning in health systems. This approach provides an ability to engineer health systems that were truly resilient to HAI increasing incidence rates and challenging to predict potentials for health system's environment of care infectivity.

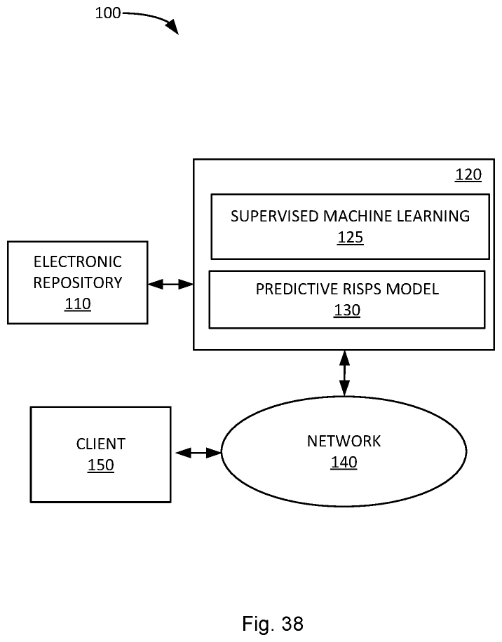

The present disclosure presents exemplary systems and methods for forecasting potential outcomes of operationally based infection control interventions in acute care settings that can enable healthcare quality and safety teams to gain meaningful and accurate insight into the possible performance safety level of environment of care infection prevention strategies through the application of a series of System Science derived mathematical models. Referring to FIG. 38, an exemplary fuzzy inference system 100 contains an electronic repository 110, a computer server 120, supervised machine learning 125, a predictive RISPS model 130, a network 140, and a client computer 150. The electronic repository 110 stores information relevant to fuzzy membership set rules and risk analysis of hospital acquired infections. The electronic repository 110 can store both structured and unstructured data. Structured data includes data stored in defined data fields, for example, in a data table. Unstructured data includes raw information, including, for example, computer readable text documents, document images, audio files, video files, and other forms of raw data.

The computer server 120 includes one or more computer processors, a memory storing the predictive model 130, supervised machine learning 125, and other hardware and software for executing the respective model. More specifically, the software may be computer readable instructions, stored on a computer readable media, such as a magnetic, optical, magneto-optical, holographic, integrated circuit, or other form of non-volatile memory. The instructions may be coded, for example, using C, C++, JAVA, SAS or other programming or scripting language. To be executed, the respective computer readable instructions are loaded into RAM associated with the computer server 120.

The predictive model 130 may be a linear regression model, a neural network, a decision tree model, or a collection of decision trees, for example, and combinations thereof. The predictive model 130 may be stored in the memory of the computer server 120, or may be stored in the memory of another computer connected to the network 140 and accessed by the computer server 120 via a network 140. The predictive model 104 preferably takes into account a large number of parameters, such as, for example, characteristics of electronic records (e.g., performance characteristics in addition with other design characteristics). The predictive model 130 may then be used by the computer server 120 to estimate the likelihood that a particular building infrastructure material will reduce the risk of infection of one or more bacterial and/or viral agents. Additionally or in the alternative, an exemplary predictive model may estimate the likelihood that a design of a building (having various infrastructure materials) will act in reducing the risk of infection of one more biological agents.

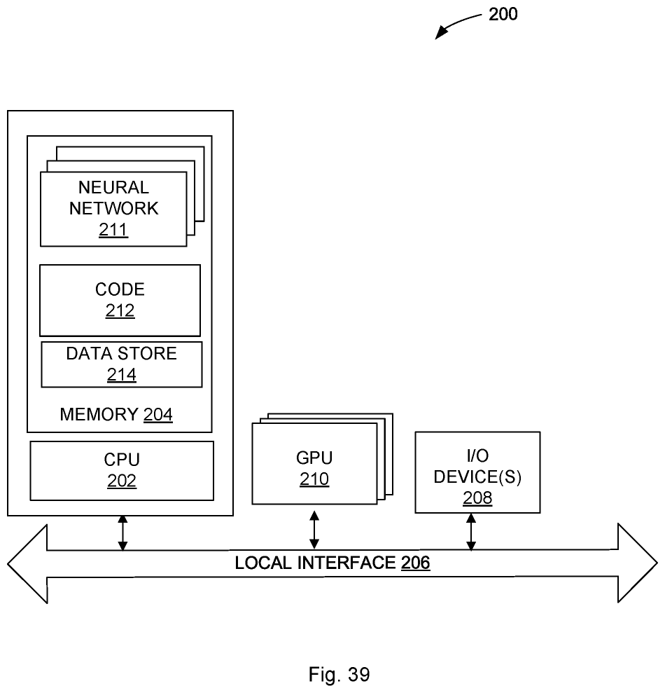

Next, FIG. 39 depicts a schematic block diagram of a computing device 200 that can be used to implement various embodiments of the present disclosure. An exemplary computing device 200 includes at least one processor circuit, for example, having a processor (CPU) 202 and a memory 204, both of which are coupled to a local interface 206, and one or more input and output (I/O) devices 208. The local interface 206 may comprise, for example, a data bus with an accompanying address/control bus or other bus structure as can be appreciated. The computing device 200 further includes Graphical Processing Unit(s) (GPU) 210 that are coupled to the local interface 206 and may utilize memory 204 and/or may have its own dedicated memory. The CPU and/or GPU(s) can perform various operations such as image enhancement, graphics rendering, image/video processing, recognition (e.g., text recognition, object recognition, feature recognition, etc.), image stabilization, machine learning, filtering, image classification, and any of the various operations described herein.

Stored in the memory 204 are both data and several components that are executable by the processor 202. In particular, stored in the memory 204 and executable by the processor 202 are code for implementing one or more neural networks 211 (e.g., artificial and/or convolutional neural network models) and code 212 for using machine learning, such as neural network models 211, for hospital acquired infection prevention data analysis. Also stored in the memory 204 may be a data store 214 and other data. The data store 214 can include an electronic repository or database relevant to computable records of hospital acquired infection prevention data analysis. In addition, an operating system may be stored in the memory 204 and executable by the processor 202. The I/O devices 208 may include input devices, for example but not limited to, a keyboard, mouse, etc. Furthermore, the I/O devices 208 may also include output devices, for example but not limited to, a printer, display, etc.

The feature or features of one embodiment may be applied to other embodiments, even though not described or illustrated, unless expressly prohibited by this disclosure or the nature of the embodiments. The phrase “configured to” means a specification or clarification of the structure or composition of an element defining what the element is, by way of a specific description of its configuration and interface with other elements or an external constraint. Functional language within such a specification is taken to be an affirmative limitation, and not a mere intended use. The disclosure has been described with reference to various specific embodiments and techniques. However, many variations and modifications are possible while remaining within the scope of the disclosure. The claims hereinbelow are to be construed as excluding abstract subject matter as judicially excluded from patent protection, and the scope of all terms and phrases is to be constrained to only include that which is properly encompassed. By way of example, if a claim phrase is amenable of construction to encompass either patent eligible subject matter and patent ineligible subject matter, then the claim shall be interpreted to cover only the patent eligible subject matter, consistent with any presumption of validity to be applied. This rule of construction overrides other claim construction predicts and linguistic presumptions. The various disclosure expressly provided herein, in conjunction with the incorporated references, are to be considered to encompass any combinations, permutations, and subcombinations of the respective disclosures or portions thereof, and shall not be limited by the various exemplary combinations specifically described herein.

### REFERENCES (EACH INCORPORATED HEREIN BY REFERENCE)

Abernathy, W. (2012) Human Performance Diagnostics: A Multidisciplinary Approach for Analysis & Improvement in Organizations. Atlanta, Ga. Performance Management Pub.
Acampora, Giovanni; Di Stefano, Bruno N.; Vitiello, Autilia (2016). “IEEE 1855™: The First IEEE Standard Sponsored by IEEE Computational Intelligence Society [Society Briefs]”. IEEE Computational Intelligence Magazine. 11 (4): 4-6. doi:10.1109/MCI.2016.2602068.
Acampora, Giovanni; Loia, Vincenzo; Lee, Chang-Shing; Wang, Mei-Hui (2013). On the Power of Fuzzy Markup Language. Studies in Fuzziness. Studies in Fuzziness and Soft Computing. 296. doi:10.1007/978-3-642-35488-5. ISBN 978-3-642-35487-8. ISSN 1434-9922.
AHRQ National Scorecard on Hospital-Acquired Conditions Updated Baseline Rates and Preliminary Results 2014-2017. (2019) www.ahrq.gov
Allen, C. R., Angeler, D. G., Garmestani, A. S., Gunderson, L. H., & Holling, C. S., Panarchy: Theory and Application. ECOSYSTEMS. Springer, New York, N.Y., 17(4):578-589, (2014).
Alvarenga, Frutuoso E Melo, and Fonseca. (2014). A critical review of methods and models for evaluating organizational factors in Human Reliability Analysis. Progress in Nuclear Energy, 75, 25-41.
Ammiche, Mustapha, Abdelmalek Kouadri, and Abderazak Bensmail. “A Modified Moving Window dynamic PCA with Fuzzy Logic Filter and application to fault detection.” Chemometrics and Intelligent Laboratory Systems 177 (2018): 100-113.
Annarelli, Alessandro, & Nonino, Fabio. (2016). Strategic and operational management of organizational resilience: Current state of research and future directions. Omega, 62, 1-18.
Anooj, P. K. (2012). Clinical decision support system: Risk level prediction of heart disease using weighted fuzzy rules. Journal of King Saud University-Computer and Information Sciences, 24(1), 27-40.
Arji, Goli, Hossein Ahmadi, Mehrbakhsh Nilashi, Tank A. Rashid, Omed Hassan Ahmed, Nahla Aljojo, and Azida Zainol. “Fuzzy logic approach for infectious disease diagnosis: A methodical evaluation, literature and classification.” Biocybernetics and biomedical engineering 39, no. 4 (2019): 937-955.
Asli, Kaveh Hariri; Aliyev, Soltan Ali Ogli; Thomas, Sabu; Gopakumar, Deepu A. (2017 Nov. 23). Handbook of Research for Fluid and Solid Mechanics: Theory, Simulation, and Experiment. CRC Press. ISBN 9781315341507.
Avci, Ozgenc, Coskuner, and Olut. (2011). Hospital acquired infections (HAI) in the elderly: Comparison with the younger patients. Archives of Gerontology and Geriatrics, 54(1), 247-250.
Axelrod, R. and Cohen, M. D., (2000). Harnessing complexity: organizational implications of a scientific frontier. New York, N.Y.: Simon and Schuster.
Azadeh, A., and Zarrin, M. (2016). An intelligent framework for productivity assessment and analysis of human resource from resilience engineering, motivational factors, HSE and ergonomics perspectives. Safety science, 89, 55-71.
Balas, Can Elmar, M. Levent Koc, and Rifat Tur. “Artificial neural networks based on principal component analysis, fuzzy systems and fuzzy neural networks for preliminary design of rubble mound breakwaters.” Applied Ocean Research 32, no. 4 (2010): 425-433.
Bansod, Nitin A; Kulkarni, Marshall; Patil, S. H. (2005). “Soft Computing-A Fuzzy Logic Approach”. In Bharati Vidyapeeth College of Engineering (ed.). Soft Computing. Allied Publishers. p. 73. ISBN 978-81-7764-632-0. Retrieved 9 Nov. 2018.
Basu, Tirthankar, and Arijit Das. “Identification of backward district in India by applying the principal component analysis and fuzzy approach: A census based study.” Socio-Economic Planning Sciences 72 (2020): 100915.
Bergstrom, J., van Winsen, R., and Henriqson, E. (2015). On the rationale of resilience in the domain of safety: A literature review. Reliability Engineering and System Safety, 141, 131-141.
Bhamra, R., Dani, S., and Burnard, K. (2011). Resilience: The concept, a literature review, and future directions. International Journal of Production Research, 49(18), 5375-5393.
Ng, Bhuvaneswari Amma. “An intelligent approach based on Principal Component Analysis and Adaptive Neuro Fuzzy Inference System for predicting the risk of cardiovascular diseases.” In 2013 Fifth International Conference on Advanced Computing (ICoAC), pp. 241-245. IEEE, 2013.
Blake, K. S., Kellerson, R. L., & Simic, A. (2007). Measuring overcrowding in housing. Washington, D.C.: Department of Housing and Urban Development, Office of Policy Development and Research.
Bondarczuk, K., & Piotrowska-Seget, Z. (2013). Molecular basis of active copper resistance mechanisms in Gram-negative bacteria. Cell biology and toxicology, 29(6), 397-405.
Boring, R. L. (2009, October). Reconciling resilience with reliability: The complementary nature of resilience engineering and human reliability analysis. In Proceedings of the Human Factors and Ergonomics Society Annual Meeting (Vol. 53, No. 20, pp. 1589-1593). Sage Calif.: Los Angeles, Calif.: Sage Publications.
Boyce, J. M. (2016). Modern technologies for improving cleaning and disinfection of environmental surfaces in hospitals. Antimicrobial Resistance and Infection Control, 5(1), 10.
Brown, A. W., & Wilson, R. B. (2018). Clostridium difficile colitis and zoonotic origins—a narrative review. Gastroenterology report, 6(3), 157-166.
Burman, M., & Fahrenwald, N., (2018). Academic nursing leadership in a rural setting: Different game, same standards. Journal of Professional Nursing, 34(2), 128-133.
Burnett, E., Lee, K., Rushmer, R., Ellis, M., Noble, M., & Davey, P. (2010). Healthcare-associated infection and the patient experience: a qualitative study using patient interviews. Journal of Hospital Infection, 74(1), 42-47.
Burton, J. (2018) The US States With The Oldest Populations. WorldAtlas. www.worldatlas.com/articles/the-us-states-with-the-oldest-population.html
Bysshe, T.; Gao, Y.; Heaney-Huls, K.; Hockenberry, J.; Hovey, L.; Laffan, A.; Lee, S.; Murphy, D.; Watts, E. (2017) Estimating the Additional Hospital Inpatient Cost and Mortality Associated with Selected Hospital-Acquired Conditions. AHRQ Publication No. 18-0011-EF. Chicago, Ill.: NORC at the University of Chicago
Campos, M. D., Zucchi, P. C., Phung, A., Leonard, S. N., & Hirsch, E. B. (2016). The activity of antimicrobial surfaces varies by testing protocol utilized. PloS one, 11(8), e0160728.
Carayon, P., and Wood, K. (2010). Patient safety—the role of human factors and systems engineering. Studies in Health Technology and Informatics, 153, 23-46.
Carayon, Pascale, Ben-Tzion Karsh, Ayse P. Gurses, Richard J. Holden, Peter Hoonakker, Ann Schoofs Hundt, Enid Montague, A. Joy Rodriguez, and Tosha B. Wetterneck. “Macroergonomics in health care quality and patient safety.” Reviews of human factors and ergonomics 8, no. 1 (2013): 4-54.
Carling, P. C., Parry, M. M., Rupp, M. E., Po, J. L., Dick, B., Von Beheren, S., and Healthcare Environmental Hygiene Study Group. (2008). Improving cleaning of the environment surrounding patients in 36 acute care hospitals. Infection Control and Hospital Epidemiology, 29(11), 1035-1041.
Cassone, M., Armbruster, C., Snitkin, E. S., Gibson, K., Manley, J., Reyes, K. C., Altamimi, S., Perri, M. B., Zervos, M. J., . . . Mody, L (2017). Relatedness of MRSA and VRE Strains Isolated from Post-acute Care Patients and Their Environment: a Longitudinal Assessment. Open Forum Infectious Diseases, 4(Suppl 1), 5639. doi:10.1093/ofid/ofx163.1696
Castlight/The Leapfrog Group. Healthcare-Associated Infections. (2018): 1-5. www.leapfroggroup.org Castro, C. E. F., and Munoz-Price, L. S. (2019). Advances in Infection Control for Clostridioides (Formerly Clostridium) difficile Infection. Current Treatment Options in Infectious Diseases, 11(1), 12-22,
Cejas, Jesús (2011). “Compensatory Fuzzy Logic”. Revista de Ingenieria Industrial. ISSN 1815-5936.
Centers for Disease Control and Prevention. (2014) Antibiotic Resistance (AR) Data Portal. Patient Safety Atlas, gis.cdc.gov/grasp/PSA/
Centers for Disease Control and Prevention. (2015) Press Release: Nearly half a million Americans suffered from Clostridium difficile infections in a single year. www.cdc.gov/media/releases/2015/p0225-clostridium-difficile.html
Centers for Disease Control and Prevention. (2016). The NHSN guide to the standardized infection ratio: a guide to the SIR. Atlanta, Ga.: CDC:
Centers for Disease Control and Prevention. [Article title], MMWR 2011; 60(Suppl):[inclusive page numbers: 58-63].
Centers for Disease Control and Prevention. Antibiotic Resistance Threats in the United States. (2013): 5-111. Claro, Tânia, Stephen Daniels, and Hilary Humphreys. “Detecting Clostridium difficile spores from inanimate surfaces of the hospital environment. Which method is best?” Journal of clinical microbiology (2014): JCM-01011.
Centers for Disease Control and Prevention. Data Summary of HA is in the US: Assessing Progress 2006-2016, (2018) www.cdc.gov/hai
Centers for Medicare and Medicaid Services (2018). Hospital-Acquired Condition Reduction Program. www.cms.gov
Cerè, G., Rezgui, Y., and Zhao, W. (2017). Critical review of existing built environment resilience frameworks: directions for future research. International journal of disaster risk reduction, 25, 173-189.
Chang, K., & Cheng, C. (2010). A risk assessment methodology using intuitionistic fuzzy set in FMEA. International Journal of System Science, 41(12), 1457-1471.
Chassin, M., and Loeb, J. (2011). The ongoing quality improvement journey: Next stop, high reliability. Health Affairs (Project Hope), 30(4), 559-568,
Chaudhuri, Arindam; Mandaviya, Krupa; Badelia, Pratixa; Ghosh, Soumya K. (2016-12-23). Optical Character Recognition Systems for Different Languages with Soft Computing: Springer, ISBN 9783319502526,
Chen, Fu-Chen, Yih-Fong Tzeng, Meng-Hui Hsu, and Wei-Ren Chen. “Combining Taguchi method, principal component analysis and fuzzy logic to the tolerance design of a dual-purpose six-bar mechanism.” Transactions of the Canadian Society for Mechanical Engineering 34, no. 2 (2010): 277-293.
Chen, S. M., & Chen, J. H. (2009). Fuzzy risk analysis based on ranking generalized fuzzy numbers with different heights and different spreads. Expert systems with applications, 36(3), 6833-6842.
Cheung, Y. B. (2007). A modified least-squares regression approach to the estimation of risk difference. American journal of epidemiology, 166(11), 1337-1344.
Chernova, I. V., S. A. Sumin, M. V. Bobyr, and S. P. Seregin. “Forecasting and diagnosing cardiovascular disease based on inverse fuzzy models.” Biomedical Engineering 49, no. 5 (2016): 263-267.
Chyderiotis, S., Legeay, C., Verjat-Trannoy, D., Le Gallou, F., Astagneau, P., and Lepelletier, D. (2018). New insights on antimicrobial efficacy of copper surfaces in the healthcare environment: a systematic review. Clinical Microbiology and Infection, 24(11), 1130-1133.
Cinner, Joshua E., Tim R. McClanahan, Nicholas A J Graham, Tim M. Daw, Joseph Maina, Selina M. Stead, Andrew Wamukota, Katrina Brown, and Ōrjan Bodin. “Vulnerability of coastal communities to key impacts of climate change on coral reef fisheries.” Global Environmental Change 22, no. 1 (2012):12-20.
Claro, T., Daniels, S., & Humphreys, H. (2014). Detecting Clostridium difficile spores from inanimate surfaces of the hospital environment: which method is best?. Journal of clinical microbiology, 52(9), 3426-3428.
Clauss-Ehiers, Caroline C. C. “Promoting Ecological Health Resilience for Minority Youth: Enhancing Health Care Access through the School Health Center.” Psychology in the Schools 40.3 (2003): 265-78. Web.
Cohen, G., Hilario, M., Sax, H., Hugonnet, S., & Geissbuhler, A. (2006). Learning from imbalanced data in surveillance of nosocomial infection. Artificial intelligence in medicine, 37(1), 7-18.
Connor, K. M., and Davidson, J. R. (2003). Development of a new resilience scale: The Connor-Davidson resilience scale (CD-RISC). Depression and anxiety, 18(2), 76-82.
Cooke, A., Smith, D., and Booth, A. (2012). Beyond PICO: the SPIDER tool for qualitative, evidence synthesis. Qualitative health research, 22(10), 1435-1443.
Dancer, S. J. (2014). Controlling hospital-acquired infection: focus on the role of the environment and new technologies for decontamination. Clinical microbiology reviews, 27(4), 665-690.
Das S, Guha D, and Dutta B (2016). “Medical diagnosis with the aid of using fuzzy logic and intuitionistic fuzzy logic”. Applied Intelligence. 45 (3): 850-867. doi:10.1007/s10489-016-0792-0. S2CID 14590409.
Dasgupta, S., Das, S., Chawan, N. S., and Hazra, A. (2015). Nosocomial infections in the intensive care unit: Incidence, risk factors, outcome and associated pathogens in a public tertiary teaching hospital of Eastern India. Indian journal of critical care medicine: peer-reviewed, official publication of Indian Soc. of Critical Care Medi., 19(1), 14-20.
David, M. Z., & Daum, R. S. (2010). Community-associated methicillin-resistant Staphylococcus aureus: epidemiology and clinical consequences of an emerging epidemic. Clinical microbiology reviews, 23(3), 616-687.
Day, S. R., Moore, C. M., Kundzins, J. R., & Sifri, C. D. (2012). Community-associated and healthcare-associated methicillin-resistant Staphylococcus aureus virulence toward Caenorhabditis elegans compared. Virulence, 3(7), 576.-582. doi:10.4161/viru.22120
de Bruin, Jeroen S., Klaus-Peter Adiassnig, Alexander Blacky, and Walter Koller. “Detecting borderline infection in an automated monitoring system for healthcare-associated infection using fuzzy logic,” Artificial intelligence in medicine 69 (2016): 33-41.
de Macedo, R. D. C. R., Jacob, E. M. O., da Silva, V. P., Santana, E. A., de Souza, A. F., Gonçalves, P., . . . and Edmond, M. B. (2012). Positive deviance: Using a nurse cat system to evaluate hand hygiene practices. American journal of infection control, 40(10), 946-950.
Deng H, Deng W, Sun X, Ye C, and Zhou X (2016). “Adaptive intuitionistic fuzzy enhancement of brain tumor MR images”. Sci. Rep. 6: 35760. Bibcode:2016NatSR . . . 635760D. doi:10.1038/srep35760. PMC 5082372. PMID 27786240.
Denoeux, Thierry, and M-H. Masson. “Principal component analysis of fuzzy data using autoassociative neural networks.” IEEE Transactions on Fuzzy Systems 12, no. 3 (2004): 336-349:
Depestel, D. D., & Aronoff, D. M. (2013). Epidemiology of Clostridium difficile infection. Journal of pharmacy practice, 26(5), 464-475. doi:10.1177/0897190013499521
Destercke, S., Guillaume, S., & Charnomordic, B. (2007). Building an interpretable fuzzy rule base from data using orthogonal least squares—application to a depollution problem. Fuzzy Sets and Systems, 158(18), 2078-2094,
Dhiman, Nitesh, and M. Sharma. “Fuzzy logic inference system for identification and prevention of Coronavirus (COVID-19).” international Journal of Innovative Technology and Exploring Engineering 9, no. 6 (2020).
Di Stefano, Bruno N. (2013). “On the Need of a Standard Language for Designing Fuzzy Systems”. On the Power of Fuzzy Markup Language. Studies in Fuzziness and Soft Computing. 296. pp. 3-15. doi:10.1007/978-3-642-35488-5_1. ISBN 978-3-642-35487-8. ISSN 1434-9922.
DiDiodato, G., and Fruchter, L. (2019). Antibiotic exposure and risk of community-associated Clostridium difficile infection: A self-controlled case series analysis. American journal of infection control, 47(1), 9-12.
Donskey, C. J. (2013). Does improving surface cleaning and disinfection reduce health care-associated infections? American journal of infection control, 41(5), S12-S19,
Dubois, D. J. (1980) Fuzzy Sets and Systems: Theory and Applications. Mathematics in Science and Engineering. V. 144 pp. 270-272. Elsevier Science.
Dubois, D., and Prade, H. (1980). Systems of linear fuzzy constraints. Fuzzy sets and systems, 3(1), 378.
Dulac, Leveson, Zipkin, Friedenthal, Cutcher-Gershenfeld, Carroll, and Barrett. (2005) “Using System Dynamics for Safety and Risk Management in Complex Engineering Systems.” Simulation Conference, 2005 Proceedings of the Winter: 10 Pp. Web.
Ehrentraut, C., Tanushi, H., Dalianis, H., & Tiedemann, J. (2012). Detection of Hospital Acquired Infections in sparse and noisy Swedish patient records. A machine learning approach using Naïve Bayes, Support Vector Machines and C, 4.
Eisawah, Guillaume, Filatova, Rook, and Jakeman. (2015). A methodology for eliciting, representing, and analysing stakeholder knowledge for decision making on complex socio-ecological systems: From cognitive maps to agent-based models. Journal of Environmental Management, 151, 500-516.
Epstein, I. (2002). Using Available Clinical Information in Practice-Based Research. Social Work in Health Care, 333-4), 15-32.
European Society of Clinical Microbiology and Infectious Diseases. (2019, April 14). Ecological study identifies potential association between antimicrobial resistance and climate change. ScienceDaily, www.sciencedaily.com/releases/2019/04/190414111454.htm
Fernandez, Rafael, Ana-Isabel Tizon, Javier Gonzalez, Pablo, Monedero, Manuela Garcia-Sanchez, Ma-Victoria de-la-Torre, Pedro Ibanez et al. “Intensive care unit discharge to the ward with a tracheostomy cannula as a risk factor for mortality: a prospective, multicenter propensity analysis,” Critical care medicine 39(10) (2011): 2240-2245,
Forrester, M. L., Pettitt, A. N., & Gibson, G. J. (2006). Bayesian inference of hospital-acquired infectious diseases and control measures given imperfect surveillance data Biostatistics, 8(2), 383-401.
Freeman, J., M. P. Bauer, Simon D. Baines, J. Corver, W. N. Fawley, B, Goorhuis, E. J. Kuijper, and M. H. Wilcox. “The changing epidemiology of Clostridium difficile infections.” Clinical microbiology reviews 23(3) (2010): 529-549,
Fronczek, A. E., Rouhana, N. A., and Kitchin, J. M. (2017). Enhancing telehealth education in nursing: Applying King's conceptual framework and theory of goal attainment. Nursing science quarterly, 30(3), 209-213.
Fuller, R. L., Averill, R. F., Muldoon, J. H., & Hughes, J. S. (2016). Comparison of the Properties of Regression and Categorical Risk-Adjustment Models. The Journal of ambulatory care management, 39(2), 157-165. doi:10.1097/JAC.0000000000000135
Furniss, Back, Blandford, Hildebrandt, and Broberg. (2011). A resilience markers framework for small teams. Reliability Engineering and System Safety, 96(1), 2-10.
Gerla, G. (2016). “Comments on some theories of fuzzy computation”. International Journal of General Systems. 45 (4): 372-392. Bibcode:2016IJGS . . . 45 . . . 372G. doi:10.1080/03081079.2015.1076403. S2CID 22577357,
Ghadimi, Ali, Mohtada Sadrzadeh, and Toraj Mohammadi. “Prediction of ternary gas permeation through synthesized PDMS membranes by using Principal Component Analysis (PCA) and fuzzy logic (FL).” journal of Membrane Science 360, no: 1-2 (2010): 509-521,
Ghaedi, M., A. M. Ghaedi, F. Abdi, M. Roosta, A, Vafaei, and A. Asghari, “Principal component analysis-adaptive neuro-fuzzy inference system modeling and genetic algorithm optimization of adsorption of methylene blue by activated carbon derived from Pistacia khinjuk.” Ecotoxicology and environmental safety 96 (2013):110-117.
Gilbert, T. (2007). Human competence: Engineering worthy performance: San Francisco, Calif.: John Wiley & Sons.
Gonzalez-Hidalgo, Manuel; Munar, Marc; Bibiloni, Pedro; Moya-Alcover, Gabriel; Craus-Miguel, Andrea; Segura-Sampedro, Juan Jose (October 2019). “Detection of infected wounds in abdominal surgery images using fuzzy logic and fuzzy sets”. 2019 International Conference on Wireless and Mobile Computing, Networking and Comm. (WiMob). Barcelona, Spain: IEEE: 99-106. doi:10.1109/WiMOB.2019.8923289. ISBN 978-1-7281-33164. S2CID 208880793.
Gould, D. J., Navaïe, D., Purssell, E., Dray, N. S.; and Creedon, S. (2018). Changing the paradigm: messages for hand hygiene education and audit from duster analysis. Journal of Hospital Infection, 98(4); 345-351.
Grass, G., Rensing, C., & Solioz, M. (2011). Metallic copper as an antimicrobial surface. Appl. Environ. Microbiol., 77(5), 1541-1547,
Grecco, C. H. D. S., Santos, I., & Carvalho, P. V. R. D. (2013). A fuzzy logic-based method to monitor organizational resilience: application in a brazilian radioactive facility.
Guillaume, S. (2001). Designing fuzzy inference systems from data: An interpretability-oriented review. IEEE transactions on fuzzy systems, 9(3), 426-443,
Habeck, C., & Brickman, A. (2018). A common statistical misunderstanding in Psychology and Neuroscience: Do we need normally distributed independent or dependent variables for linear regression to work? BioRxiv, Apr. 24, 2018.
Hague, M., Sartelli, M., McKimm, J., & Abu Bakar, M. (2018). Health care-associated infections—an overview. Infection and drug resistance, 11, 2321-2333. doi:10.2147/IDR.S177247
Health Resources & Services Administration (2016) Shortage Designation: Medically Underserved Areas and Populations (MUA/Ps). bhw.hrsa.gov
Heron M. (2018) Deaths: Leading causes for 2016. National Vital Statistics Reports; vol 67 no 6.
Higuchi, Shoji, and Yutaka Hata. “Fuzzy logic approach to health checkup data analysis.” In 2014 World Automation Congress (WAC), pp. 388-393, IEEE, 2014.
Holling, C. (1973). Resilience and Stability of Ecological Systems. Ann. Rev, of Ecology and Systematics, 4; 1-23.
Hollnagel, E. (1998). Cognitive reliability and error analysis method (CREAM). Elsevier.
Hollnagel, E. (2013). Resilience engineering and the built environment. Building Research and Information, 1-8.
Hollnagel, E., Braithwaite, J., and Wears, R. L. (Eds.). (2013). Resilient health care. Ashgate Publishing, Ltd.
Hollnagel, E.; Woods, D; and Leveson, N. Resilience Engineering: Concepts and Precepts.
Husseini, S., Barker; K., and Ramirez-Marquez, J. E. (2016). A review of definitions and measures of system resilience. Reliability Engineering and System Safety, 145, 47-61.
Hou, Kang, Wendong Tao, Liming Wang, and Xuxiang Li. “Study on hierarchical transformation mechanisms of regional ecological vulnerability and its applicability.” Ecological indicators 114 (2020): 106343.
Hsieh; H. F., and Shannon, S. E. (2005). Three approaches to qualitative content analysis. Qualitative health research, 15(9), 1277-1288.
Huang, S. S., Singh, R., McKinnell, J. A., Park, S., Gombosev, A., Eells, S. J., . . . & Bolaris, M. A, (2019). Decolonization to reduce post discharge infection risk among MRSA carriers. New England Journal of Medicine, 380(7), 638-650.
Hugonnet, S., Chevrolet, J. and Pittet, D. (2007). The effect of workload on infection risk in critically it patients. Critical Care Medicine, 35(1), 76-81, doi: 10.1097/01.CCM.0000251125.08629.3F.
Imhoff, M. L., Zhang, P., Wolfe, R. E., & Bounoua, L (2010). Remote sensing of the urban heat island effect across biomes in the continental USA. Remote sensing of environment, 114(3), 504-513.
Immergluck, L. C., Leong, T., Matthews, K., Malhotra, K., Parker, T. C., At, F., . . . and Rust, G. S. (2019). Geographic surveillance of community associated MRSA infections in children using electronic health record data BMC infectious diseases, 19(1), 170,
Institute of Medicine (US) Committee on Assuring the Health of the Public in the 21st Century. The Future of the Public's Health in the 21st Century. Washington (D.C.): National Academies Press (US); 2002. 3, The Governmental Public Health Infrastructure. Available from: www.ncbi.nlm.nih.gov/books/NBK221231/
Jaffe, S., (2015). Aging in rural America. Health Affairs: Entry Point. www.healthaffairs.org
James, G., Witten, Daniela, author, Hastie, Trevor author, & Tibshirani, Robert author. (2013). An introduction to statistical learning: With applications in R (Uncorrected ed., Springer texts in statistics. 103).
Kamadi, VSRP Varma, Appa Rao Allam, and Sita Mahalakshmi Thummala. “A computational intelligence technique for the effective diagnosis of diabetic patients using principal component analysis (PCA) and modified fuzzy SLIQ decision tree approach.” Applied Soft Computing 49 (2016): 137-145.
Karwowski W., Grobelny J., Lee W., Yang Y N. (1999) Fuzzy Sets in Human Factors and Ergonomics. In: Zimmermann H J. (eds) Practical Applications of Fuzzy Technologies. The Handbooks of Fuzzy Sets Series, vol 6. Springer, Boston, Mass.
Kaur, D. C., and Chate, S. S. (2015). Study of antibiotic resistance pattern in methicillin resistant Staphylococcus aureus with special reference to newer antibiotic. Journal of global infectious diseases, 7(2), 78.
Kavanagh, K., Abusalem, S., and Calderon, L. (2017). The incidence of MRSA infections in the United States: is a more comprehensive tracking system needed? Antimicrobial Resistance and Infection Control, 6(1), 1-6.
Khademi, F., Akbari, M., Jamal, S. M., & Nilo, M. (2017). Multiple linear regression, artificial neural network, and fuzzy logic prediction of 28 days compressive strength of concrete. Frontiers of Structural and Civil Eng., 11(1), 90-99.
Khasawneh, M. T., Bowling, S. R., Jiang, X., Gramopadhye, A. K., and Melloy, B. J. (2003). A model for predicting human trust in automated systems. Origins, 5,
Kim, G., & Zhu, N. A., (2017). Community-acquired Clostridium difficile infection. Canadian family physician Medecin de famille canadien, 63(2), 131-132.
King, I. M. (2007). King's conceptual system, theory of goal attainment, and transaction process in the 21st century. Nursing Science Quarterly, 20(2), 109-111.
Klir, G. and Yuan, B. (1995). Fuzzy sets and fuzzy logic (Vol. 4). New Jersey: Prentice Hall.
Kochan, C. G., and Nowicki, D. R. (2018). Supply chain resilience: a systematic literature review and typological framework. International Journal of Physical Distribution and Logistics Management, 48(8), 842-865.
Kok, O'sullivan, & Gilbert. (2011). Feedback to clinicians on preventable factors can reduce hospital onset Staphylococcus aureus bacteraemia rates. Journal of Hospital infection, 79(2), 108-114.
Konstandinidou, Nivolianitou, Kiranoudis, and Markatos. (2006). A fuzzy modeling application of CREAM methodology for human reliability analysis. Reliability Engineering and System Safety, 91(6), 706716.
Kosko, Bad. “Fuzziness vs. Probability” (FDF). University of South California. Retrieval 9 Nov. 2018.
Kovach, R, (2015) Using Planning, Checklists, Other Tools to it Resilience in Existing Buildings: Facilitiesnet. www.facilitiesnet.com/emergencypreparedness/article/-16051
Kramer, Axel, Ingeborg Schwebke, and Günter Kampf. “How long do nosocomial pathogens persist on inanimate surfaces? A systematic review.” BMC infectious diseases 6.1 (2006):130,
Kurashige, E., Oie, S., and Furukawa, H. (2016). Contamination of environmental surfaces by methicillin-resistant Staphylococcus aureus (MRSA) in rooms of inpatients with MRSA-positive body sites. brazilian journal of microbiology, 47(3), 703-705.
Lessa, Fernanda C., Yi Mu, Wendy M. Bamberg, Zintars G. Beldavs, Ghinwa K. Dumyati, John R. Dunn, Monica M. Farley et al. “Burden of Clostridium difficile infection in the United States.” New England Journal of Medicine 372, no. 9 (2015): 825-834.
Latham, G. P., & Locke, E. A. (2007). New developments in and directions for goal-setting research. European Psychologist, 12(4), pp. 290-300.
Leite, C. R., Sizilio, G. R., Neto, A. D., Valentim, R. A., and Guerreiro, A. M. (2011). A fuzzy model for processing and monitoring vital signs in ICU patients. Biomedical engineering online, 10(1), 68.
Lessa, F. C., Mu, Y., Bamberg, W. M., Beldavs, Z. G., Dumyati, G. K., Dunn, J. and Wilson,
Lewis, H. W. (2013). The foundations of fuzzy control (Vol. 10). Springer Science and Business Media.
Li, M., Shi, X., Li, X., Ma, W., He, J., & Liu, T. (2019). Sensitivity of disease cluster detection to spatial scales: an analysis with the spatial scan statistic method. International Journal of Geographical Information Science, 1-28.
Lin K P, Chang H F, Chen T L, Lu Y M, and Wang C H (2016). “Intuitionistic fuzzy C-regression by using least squares support vector regression”. Expert Systems with Applications. 64: 296-304. doi:10.1016/j.eswa.2016.07.040.
Linkov, I., Anklam, E., Collier, Z., DiMase, A., & Renn, D. (2014). Risk-based standards: Integrating top-down and bottom-up approaches. Environment Systems and Decisions, 34(1), 134-137.
MacFadden, D. R., McGough, S. F., Fisman, D., Santillana, M., & Brownstein, J. S. (2018). Antibiotic resistance increases with local temperature. Nature climate change, 8(6), 510.
MacIntyre, C. R., & Bui, C. M. (2017). Pandemics, public health emergencies, and antimicrobial resistance—putting the threat in an epidemiologic and risk analysis context. Archives of public health=Archives beiges de sante publique, 75, 54. doi:10.1186/s13690-017-0223-7
Macintyre, S., Ellaway, A., & Cummins, S. (2002). Place effects on health: how can we conceptualise, operationalise and measure them? Social Science & Medicine, 55(1), 125-139. doi.org/10.1016/S0277-9536(01)00214-3
Mackun, P. J., Wilson, S., Fischetti, T. R., & Goworowska, J. (2011). Population distribution and change: 2000 to 2010. US Department of Commerce, Economics and Statistics Administration, US Census Bureau.
Magill, Shelley S., Jonathan R, Edwards, Wendy Bamberg, Zintars G. Beldavs, Ghinwa Dumyati, Marion A. Kainer, Ruth Lynfield et al. “Multistate point-prevalence survey of health care-associated infections.” New England Journal of Medicine 370, no. 13 (2014):1198-1208.
Maina, Joseph, Valentijn Venus, Timothy R. McClanahan, and Mebrahtu Ateweberhan. “Modelling susceptibility of coral reefs to environmental stress using remote sensing data and GIS models.” Ecological modelling 212(3-4) (2008): 180-199.
Makary M A; Daniel M. (May 2016) Medical error—the third leading cause of death in the US. BMJ. 2016; 353:i2139
Mamdani, E. H. and S. Assilian, “An experiment in linguistic synthesis with a fuzzy logic controller,” international Journal of Man-Machine Studies, Vol. 7, No. 1, pp. 1-13, 1975
Marengo, Emilio, Elisa Robotti, Pier Giorgio Righetti, and Francesca Antonucci, “New approach based on fuzzy logic and Principal Component Analysis for the classification of two-dimensional maps in health and disease: application to lymphomas.” Journal of Chromatography A 1004, no. 1-2 (2003):13-28.
Mares, Milan (2006). “Fuzzy Sets”. Scholarpedia. 1 (10): 2031. Bibcode:2006SchpJ . . . 1.2031M. doi:10.4249/scholarpedia.2031.
Martens, E., and Demain, A. L. (2017). The antibiotic resistance crisis, with a focus on the United States. The Journal of antibiotics, 70(5), 520.
Maseleno, Andino, Md Mahmud Hasan, Norjaidi Tuah, and Muhammad Muslihudin. “Fuzzy Logic and Dempster-Shafer belief theory to detect the risk of disease spreading of African Trypanosomiasis,” In 2015 Fit International Conference on Digital Information Processing and Communications (ICDIPC), pp, 153-158. IEEE, 2015.
Matzenberger, J. (2013). A novel approach to exploring the concept of resilience and principal drivers in a learning environment. Multicultural Education and Technology Journal, 7(2), 192-206. doi:dx.doi.org/10.1108/17504971311328071Macintosh, R., MacLean, D, and Burns, H. (2007). Health in Organization: Towards a Process-Based View. Journal of Management Studies, 44(2), 206-221.
McDonald, L. C., Owings, M., & Jernigan, D. B. (2006): Clostridium difficile infection in patients discharged from US short-stay hospitals, 1996-2003. Emerging infectious diseases, 12(3), 409.
McGlone, S. M., Bailey, R. R., Zimmer, S. M., Popovich, M. J., Tian, Y., Ufberg, P., . . . & Lee, B. Y. (2012). The economic burden of Clostridium difficile. Clinical Microbiology and Infection, 18(3), 282-289.
McGuckin, M., and Govednik, J. (2015). A review of electronic hand hygiene monitoring: considerations for hospital management in data collection, healthcare worker supervision, and patient perception. Journal of Healthcare Management, 60(5), 348-361.
McClanahan, T. R., J. E. Ginner, J. Maina, N. A. J. Graham, I. M. Daw, S. M. Stead, A. Wamukota et at “Conservation action in a changing climate.” Conservation letters 1, no. 2 (2008): 53-59.
Mendel, J. M. (1995): Fuzzy logic systems for engineering: a tutorial. Proceedings of the IEEE, 83(3), 345-377.
Merlo, Ohlsson, Chaix, Lichtenstein, Kawachi, & Subramanian. (2013). Revisiting causal neighborhood effects on individual ischemic heart disease risk: A quasi-experimental multilevel analysis among Swedish siblings. Social Science & Medicine, 76(1), 39-46.
Muller, G. Fuzzy architecture assessment for critical infrastructure resilience. Procedia Comp: Sci., 12, 367-372 (2012)
Musulin, E., I. Yélamos, and L. Puigjaner. “Integration of principal component analysis and fuzzy logic systems for comprehensive process fault detection and diagnosis.” Ind. & eng. chemistry research 45, no. 5 (2006): 1739-1750.
Naggie, S., et al. “Community-associated Clostridium difficile infection: experience of a veteran affairs medical center in southeastern USA,” Infection 38.4 (2010): 297-300.
Nasirzadeh, F., Afshar, A., Khanzadi, M., and Howick, S. (2008). Integrating system dynamics and fuzzy logic modeling for construction risk management. Construction Management and Economics, 26(11), 1197-1212.
NCHS Data Brief, no 328. Hyattsville, Md.: National Center for Health Statistics. 2018.
Ngam, C., Hundt, A. S., Haun, N., Carayon, P., Stevens, L., and Safdar, N. (2017). Barriers and facilitators to Clostridium difficile infection prevention: A nursing perspective. Am. J of infection control, 45(12), 1363-1363.
Nivolianitou, Z., and Konstantinidou, M. (2018). A Fuzzy Modeling Application for Human Reliability Analysis in the Process Industry. In Human Factors and Reliability Engineering for Safety and Security in Critical Infrastructures (pp. 109-154). Springer, Cham.
Norris, Fran H., Stevens, Susan P., Pfefferbaum, Betty, Wyche, Karen F., and Pfefferbaum, Rose L. (2008). Community Resilience as a Metaphor, Theory, Set of Capacities, and Strategy for Disaster Readiness. American Journal of Community Psychology, 41(1-2), 127-150.
Novák, V (2005). “Are fuzzy sets a reasonable tool for modeling vague phenomena?”. Fuzzy Sets and Systems. 156 (3): 341-348. doi:10.1016/j.fss.2005.05.029.
Novák, V.; Pertlieva, I.; Močkoř, J. (1999). Mathematical principles of fuzzy logic. Dordrecht: Kluwer Academic. ISBN 978-0-7923-8595-0.
O'Neill, L., Park, S. H., and Rosinia, F. (2018). The role of the built environment and private rooms for reducing central line-associated bloodstream infections. PloS one, 13(7), e0201002.
Obenshain, M. K. (2004). Application of data mining techniques to healthcare data. Infection Control & Hospital Epidemiology, 25(8), 690-695.
Ouyang, M. (2014). Review on modeling and simulation of interdependent critical infrastructure systems. Reliability engineering and System safety, 121, 43-60.
Panarchy: theory and application. Ecosystems, 17(4), 578-589.
Patriarca, R., Bergstrom, J., Di Gravio, G., and Costantino, F. (2018). Resilience engineering: Current status of the research and future challenges. Safety Science, 102, 79-100.
Pei, Duan, Shifeng Fang, Lu Lin, Zhihao Qin, and Xiaoyan Wang. “Methods and applications for ecological vulnerability evaluation in a hyper-arid oasis: a case study of the Turpan Oasis, China.” Environmental Earth Sciences 74, no. 2 (2015): 1449-1461.
Pelletier, Francis Jeffry (2000). “Review of Metamathematics of fuzzy logics” (PDF). The Bulletin of Symbolic Logic. 6 (3): 342-346. doi:10.2307/421060. JSTOR 421060. Archived (PDF) from the original on 2016-03-03.
Petti, S., De Giusti, M., Moroni, C., and Polimeni, A. Long-term survival curve of methicillin-resistant Staphylococcus aureus on clinical contact surfaces in natural-like conditions. Am. J of infection control, 40(10), 1010-1012 (2012).
Pu, Yuanyuan, Derek Apel, and Huawei Xu. “A principal component analysis/fuzzy comprehensive evaluation for rockburst potential in kimberlite.” Pure and Applied Geophysics 175, no. 6 (2018): 2141-2151.
Ramanujam, R., K. Venkatesan, Vimal Saxena, Rachit Pandey, T. Harsha, and Gurusharan Kumar. “Optimization of machining parameters using fuzzy based principal component analysis during dry turning operation of inconel 625—A hybrid approach.” Procedia Engineering 97 (2014): 668-676.
Ratcliffe, M., Burd, C., Holder, K., & Fields, A. (2016). Defining rural at the US Census Bureau. American community survey and geography brief, 1-8.
Razin, Mir Reza Ghaffari, and Behzad Voosoghi. “Ionosphere time series modeling using adaptive neuro-fuzzy inference system and principal component analysis.” GPS Solutions 24, no. 2 (2020):1-13.
Reason, J. (2016). Managing the risks of organizational accidents. Routledge.
Righi, Saurin, and Wachs. (2015). A systematic literature review of resilience engineering: Research areas and a research agenda proposal. Reliability Engineering and System Safety, 141(C), 142-152.
Roberts, R. R., Hota, B., Ahmad, I., Scott, R. D., Foster, S. D., Abbasi, F., . . . & Naples, J. (2009). Hospital and societal costs of antimicrobial-resistant infections in a Chicago teaching hospital: implications for antibiotic stewardship. Clinical infectious diseases, 49(8), 1175-1184.
Ross, T. J. (2009). Fuzzy logic with engineering applications. John Wiley and Sons.
Rothblum, A. M. (2000, October). Human error and marine safety. In National Safety Council Congress and Expo, Orlando, Fla. (p. 7).
Rutala, W. A., & Weber, D. J. (2013). Disinfectants used for environmental disinfection and new room decontamination technology. American journal of infection control, 41(5), S36-S41.
Sabokbar, Hassanali Faraji, Majid Shadman Roodposhti, and Esmaeil Tazik. “Landslide susceptibility mapping using geographically-weighted principal component analysis.” Geomorphology 226 (2014): 15-24.
Sahoo, K. C., Sahoo, S., Marrone, G., Pathak, A., Lundhorg, C. S., & Tamhankar, A. J. (2014). Climatic factors and community-associated methicillin-resistant Staphylococcus aureus skin and soft-tissue infections—a time-series analysis study. International journal of environmental research and public health, 11(9), 8996-9007. doi:10.3390/ijerph110908996
Schaier, M., Wendt, C., Zeier, M., and Ritz, E. (2004). Clostridium difficile diarrhea in the immunosuppressed patient—update on prevention and management. Nephrology Dialysis Transplantation, 19(10), 2432-2436.
Schoch-Spana, M., Waldhorn, R. Shearer, M. Inglesby, T (2018) “A Framework for Healthcare Disaster Resilience: A View to the Future,” John Hopkins Center for Health Security. www.centerforhealthsecurity.org
Schultz, Katherine, Emily Sickbert-Bennett, Ashley Marx, David J. Weber, Lauren M. DiBiase, Stacy Campbell-Bright, Lauren E. Bale et al. “Preventable patient harm: a multidisciplinary, bundled approach to reducing Clostridium difficile infections while using a glutamate dehydrogenase/toxin immunochromatographic assay/nucleic acid amplification test diagnostic algorithm.” Journal of clinical microbiology 56(9), e00625-18 (2018).
Seager, Thomas P., Susan Spierre Clark, Daniel A. Eisenberg, John E. Thomas, Margaret M. Hinrichs, Ryan Kofron, Camilla Nørgaard Jensen, Lauren R. McBurnett, Marcus Snell, and David L. Alderson. “Redesigning resilient infrastructure research.” In Resilience and risk: Methods and Application in Environment, Cyber and Social Domains, Chapter: 3, pp. 81-119. Springer, Dordrecht, 2017.
Shah, N., Castro-Sanchez, E., Charani, E., Drumright, L. N., and Holmes, A. H. (2015). Towards changing healthcare workers' behaviour: a qualitative study exploring non-compliance through appraisals of infection prevention and control practices. Journal of Hospital infection, 90(2), 126-134.
Shandoosti, Hamid Reza, and Hassan Ghassemian. “Combining the spectral PCA and spatial PCA fusion methods by an optimal filter.” Information Fusion 27 (2016): 150-160.
Shao, Huaiyong, Meng Liu, Qiufang Shao, Xiaofei Sun, Jinhui Wu, Zhiying Xiang, and Wunian Yang. “Research on eco-environmental vulnerability evaluation of the Arming River Basin in the upper reaches of the Yangtze River.” Environmental earth sciences 72, no. 5 (2014):1555-1566.
Shaughnessy, M., Micielli, R., Depestel, D., Arndt, J., Strachan, C., Welch, K., and Chenoweth, C. (2011). Evaluation of Hospital Room Assignment and Acquisition of Clostridium difficile Infection. Infection Control and Hospital Epidemiology, 32(3), 201-206.
Shekelle, Paul G., Robert M. Wachter, Peter J. Pronovost, K. Schoelles, K. M. McDonald, S. M. Dy, K. Shojania et al. “Making health care safer II: an updated critical analysis of the evidence for patient safety practices.” Evidence report/technology assessment 211 (2013):1-045.
Sifri, Burke, and Enfield, (2016). Reduced health care-associated infections in an acute care community hospital using a combination of self-disinfecting copper-impregnated composite hard surfaces and linens. AJIC: American Journal of Infection Control, 44(12), 1565-1571
Sikula, N., Mancillas, R., Linkov, J., and McDonagh, W. (2015). Risk management is not enough: A conceptual model for resilience and adaptation-based vulnerability assessments. Env. Systems and Decisions, 35(2), 219-228.
Smith, E., and Hoff, J. (2000). Cognitive fuzzy modeling for enhanced risk assessment in a health care institution. IEEE Intelligent Systems and Their Applications, 15(2), 6975.
Smith, S. J., Young, V., Robertson, C., and Dancer, S. J. (2012). Where do hands go? An audit of sequential hand-touch events on a hospital ward. Journal of hospital infection, 80(3), 206-211.
Solis-Hernandez, P., Vidales-Reyes, M., Garza-Gonzalez, E., Guajardo-Alvarez, G., Chavez-Moreno, S., and Camacho-Ortiz, A. (2015). Hospital-Acquired infections in Elderly Versus Younger Patients in an Acute Care Hospital. International Journal of Infection, 3(1), international Journal of Infection, Nov. 15, 2015, Vol. 3(1).
Sousa, A. F. L. D., Queiroz, A. A. F. L. N., Oliveira, L. B. D., Moura, L. K. B., Andrade, D. D., Watanabe, E., and Moura, M. E. B. (2017). Deaths among the elderly with ICU infections. Rev. brasileira de enfermagem, 70(4), 733-739.
Spigaglia, Patrizia. “Recent advances in the understanding of antibiotic resistance in Clostridium difficile infection,” Therapeutic advances in infectious disease 3.1 (2016): 23-2,
Stanford Encyclopedia of Philosophy, “Fuzzy Logic”, Bryant University. 2006-07-23. Retrieved 2008-09-30.
Stone, P. W., Clarke, S., Cimiotti, J., and Correa-de-Araujo, R. (2004). Nurses' Working Conditions: implications for Infectious Disease, Emerging Infectious Diseases, 10(11), 1984-1989. dx.doi.org/10.3201/eid1011.040253,
Sugeno, M., Industrial applications of fuzzy control, Elsevier Science Pub. Co., 1985.
Suleyman, G., Alangaden, G., and Bardossy, A. The Role of Environmental Contamination in the Transmission of Nosocomial Pathogens and Healthcare-Associated Infections. Curr Infectious Disease Rep, 20(6), 1-11 (2018).
Sutcliffe, A. (2006, June). Trust: From cognition to conceptual models and design. In International Conference on Advanced Information Systems Engineering (pp. 3-17). Springer, Berlin, Heidelberg.
Sydnor, E. R., & Perl, T. M. (2011). Hospital epidemiology and infection control in acute-care settings. Clin. microbiology reviews, 24(1), 141-173. doi:10.1128/CMR.00027-10
Tadić, D., Aleksić, A., Stefanović, M., & Arsovski, S. (2014). Evaluation and ranking of organizational resilience factors by using a two-step fuzzy AHP and fuzzy TOPSIS. Mathematical Problems in Engineering, 2014.
Taylor, M., & Chaplin, S. (2013). The economic assessment of an environmental intervention: Discrete deployment of copper for infection control in ICUs. Value in Health, 16(7), A353.
The American National Standards Institute (ANSI) and The American Society of Safety Engineers (ASSE) (2016) Prevention through Design: Guidelines for Addressing Occupational Hazards and Risks in Design and Redesign Processes. ANSI/ASSE Z590.3-2011 (R2016). Park Ridge, Ill.: American Society of Safety Engineers.
The American National Standards Institute (ANSI) and The American Society of Safety Engineers (ASSE) (2017) Occupational Health and Safety Management Systems. ANSI/ASSE Z10-2012 (R2017). Park Ridge, Ill.: American Society of Safety Engineers.
The Joint Commission, (2018). Facts about Hospital Accreditation, Topic Details. www.jointcommission.org/facts_about_hospital_accreditation/
The National Healthcare Safety Network (2019). Long Term Care Facility Component Tracking Infections in Long-term Care Facilities. Division of Healthcare Quality Promotion National Center for Emerging and Zoonotic Infectious Diseases Atlanta, Ga., USA.
Tran, Balchanos, Domerçant, and Mavris. “A Framework for the Quantitative Assessment of Performance-based System Resilience.” Reliability Engineering and System Safety 158 (2017): 73-84. Web.
Trochim, W. M. (2006) The Research Methods Knowledge Base, 2nd Edition. Internet WWW page, at URL: www.socialresearchmethods.net/kb/
Ubale, A., & Sananse, S. (2016). A comparative study of fuzzy multiple regression model and least square method. International Journal of Applied Research, 2(7), 11-15.
United States Nuclear Regulatory Commission (2019). NRC Library, Basic References: Glossary-Design-basis accident. www.nrc.gov.
UT Southwestern Medical Center, (2018, May 15). Superbug MRSA infections less costly, but still deadly. ScienceDaily www.sciencedaily.com/releases/2018/05/180515131543.htm
Valiant, Leslie (2013). Probably Approximately Correct: Nature's Algorithms for Learning and Prospering in a Complex World. New York: Basic Books. ISBN 978-0465032716.
Ventola, C. L. The antibiotic resistance crisis: part 1: causes and threats. Pharm. and therapeutics, 40(4), 277 (2015)
Veri, Francesco (2017), “Fuzzy Multiple Attribute Conditions in fsQCA: Problems and Solutions”. Sociological Methods & Research, 49 (2): 312-355. doi:10.1177/0049124117729693. S2CID 125146607.
Vlachos I K, Sergiadis G D (2007). “Intuitionistic fuzzy information—applications to pattern recognition”. Pattern Recognition Letters. 28 (2): 197-206. doi:10.1016/j.patrec.2006.07.004.
von Dessauer, B., Navarrete, M. S., Benadof, D., Benavente, C., & Schmidt, M. G, (2016). Potential effectiveness of copper surfaces in reducing health care—associated infection rates in a pediatric intensive and intermediate care unit: A nonrandomized controlled trial. American journal of infection control, 44(8), e133-e139.
Wang, Wen-Ting, and Hsin-Cheng Huang. “Regularized principal component analysis for spatial data,” Journal of Computational and Graphical Statistics 26, no. 1 (2017):14-25,
Weber, D., Rutala, W., Miller, M., Huslage, K., & Sickbert-Bennett, E. (2010). Role of hospital surfaces in the transmission of emerging health care-associated pathogens: Norovirus, Clostridium difficile, and Acinetobacter species. AJIC: American Journal of Infection Control, 38(5), S25-S33. doi.org/10.1016/j.ajic.2010.04.196
Weber, David J., Deverick Anderson, and William A. Rutala: “The role of the surface environment in healthcare-associated infections.” Current opinion in infectious diseases 26.4 (2013): 338-344.
Weng, M. K., Adkins, S. H., Bamberg, W., Farley, M. M., Espinosa, C. C., Wilson, L., & Hancock, E. B. (2019). Risk factors for community-associated Clostridioides difficile infection in young children. Epidemiology & Infection, 147.
Wesson, I., P., Gualandi, N., Dumyati, G., Harrison, L., Lesher, L., . . . Ahern, J. (2017). Socioeconomic Factors Explain Racial Disparities in Invasive Community-Associated Methicillin-Resistant Staphylococcus aureus Disease Rates. Clinical Infectious Diseases: A Infectious Diseases Society of America, 64(5), 597-604.
Weston, J., Mukherjee, S., Chapelle, O., Pontil, M., Poggio, T., & Vapnik, V. (2001). Feature selection for SVMs. In Adv. in neural information processing systems (pp. 668-674).
Wierman, Mark J. “An introduction to the Mathematics of Uncertainty: including Set Theory, Logic, Probability, Fuzzy Sets, Rough Sets, and Evidence Theory” (PDF). Creighton University. (2012).
Woods, D. D. (2015). Four concepts for resilience and the implications for the future of resilience engineering. Reliability Engineering and System Safety, 141, 5-9.
Woods, D. D.; Dekker, S., Cook, R., Johannesen, L., & Saler, N. (2017). Behind human error. CRC Press.
World Health Organization, (2011). Report on the burden of endemic health care-associated infection worldwide.
Yakob, L., Riley, I. V., Paterson, D. L., and Clements, A. C. (2013). Clostridium difficile exposure as an insidious source of infection in healthcare settings: an epidemiological model. BMC infectious diseases, 13, 376. doi:10.1186/1471-2334-13-376
Yanase, Juri; Triantaphyllou, Evangelos (2019). “A Systematic Survey of Computer-Aided Diagnosis in Medicine: Past and Present Developments”. Expert Systems with Applications. 138: 112821. doi:10.10.1016/j.eswa.2019.112821.
Yanase, Juri; Triantaphyilou, Evangelos (2019). “The Seven Key Challenges for the Future of Computer-Aided Diagnosis in Medicine”. International Journal of Medical Informatics. 129: 413-422. doi:10.1016/j.ijmedinf.2019.06.017. PMID 31445285.
Yanke, Eric, et al. “Understanding the current state of infection prevention to prevent Clostridium difficile infection: a human factors and systems engineering approach.” American journal of infection control 43.3 (2015): 241-247.
Yeh, Yun-Chi. “An Analysis of ECG for Determining Heartbeat Case by Using the Principal Component Analysis and Fuzzy Logic.” International Journal of Fuzzy Systems 14, no. 2 (2012).
Yen, J., & Wang, L. (1999). Simplifying fuzzy rule-based models using orthogonal transformation methods. IEEE Transactions on Systems, Man, and Cybernetics, Part B (Cybernetics), 29(1), 13-24.
York Health Economic Consortium (2013) “An Economic Evaluation of the Use of Copper in Reducing the Rate of Healthcare Associated Infections in the UK.” www.antimicrobialcopper.org
Zadeh, L. A. “Fuzzy Logic=Computing with words”. IEEE Transactions on Fuzzy Systems, 4, pp. 103-111 (1996).
Zadeh, L. A.; et al. (1996). Fuzzy Sets, Fuzzy Logic, Fuzzy Systems. World Sci. Pr. ISBN 978-981-02-2421-9.
Zadeh, L. A. (1965). “Fuzzy sets”. Information and Control. 8 (3): 338-353. doi:10.1016/s0019-9958(65)90241-x.
Zaitsev, D. A.; Sarbei, V. G.; Sleptsov, A. I. (1998). “Synthesis of continuous-valued logic functions defined in tabular form”. Cybernetics and Systems Analysis. 34 (2):190-195. doi:10.1007/BF02742068. S2CID 120220846.
Zhang, & Chu. (2011). Risk prioritization in failure mode and effects analysis under uncertainty. Expert Systems with Applications, 38(1), 206-214.
Zhang, D., Prabhu, V. S., & Marcella, S. W. (2018). Attributable healthcare resource utilization and costs for patients with primary and recurrent Clostridium difficile infection in the United States, Clinical Infectious Diseases, 66(9), 1326-1332.

The following patents provide disclosure of technologies useful in the implementation of the invention. Each of these is expressly incorporated herein by reference in its entirety: U.S. Pat. Nos. 4,860,213; 4,875,184; 4,930,084; 4,939,648; 5,058,033; 5,068,664; 5,089,978; 5,130,936; 5,136,685; 5,189,619; 5,222,155; 5,228,111; 5,243,687; 5,245,695; 5,247,472; 5,280,565; 5,321,639; 5,347,615; 5,351,200; 5,377,308; 5,401,949; 5,412,752; 5,425,131; 5,459,816; 5,479,580; 5,481,700; 5,485,550; 5,499,319; 5,524,176; 5,544,256; 5,544,281; 5,572,597; 5,579,439; 5,602,966; 5,606,646; 5,614,116; 5,642,467; 5,664,066; 5,666,481; 5,673,365; 5,687,290; 5,694,590; 5,706,497; 5,710,868; 5,737,519; 5,754,738; 5,768,137; 5,774,357; 5,802,204; 5,828,812; 5,830,135; 5,835,901; 5,841,651; 5,867,386; 5,875,108; 5,880,830; 5,901,246; 5,903,454; 5,914,721; 5,920,477; 5,991,709; 6,073,262; 6,078,911; 6,081,750; 6,141,553; 6,172,679; 6,205,438; 6,205,439; 6,216,086; 6,236,365; 6,252,544; 6,272,476; 6,295,514; 6,390,979; 6,400,996; 6,418,424; 6,421,612; 6,429,812; 6,430,544; 6,434,490; 6,453,246; 6,532,454; 6,571,227; 6,618,647; 6,629,086; 6,640,145; 6,671,627; 6,678,619; 6,701,312; 6,721,453; 6,754,647; 6,791,472; 6,834,239; 6,850,252; 6,870,956; 6,882,992; 6,952,181; 6,971,383; 6,976,016; 7,006,881; 7,039,621; 7,043,293; 7,054,757; 7,113,640; 7,116,716; 7,117,187; 7,127,120; 7,136,710; 7,139,739; 7,158,970; 7,164,798; 7,188,055; 7,242,988; 7,257,586; 7,260,261; 7,268,700; 7,271,737; 7,274,741; 7,295,608; 7,298,289; 7,346,473; 7,865,508; 7,389,208; 7,400,761; 7,416,524; 7,444,018; 7,451,005; 7,471,827; 7,512,571; 7,529,722; 7,548,936; 7,587,280; 7,590,589; 7,599,918; 7,610,251; 7,620,672; 7,650,319; 7,668,797; 7,676,445; 7,711,670; 7,743,078; 7,812,766; 7,813,822; 7,872,104; 7,873,479; 7,904,187; 7,908,091; 7,933,612; 7,933,855; 7,937,402; 7,943,328; 7,966,078; 7,974,714; 7,983,732; 7,986,372; 7,987,003; 8,018,792; 8,027,349; 8,031,060; 8,032,477; 8,036,265; 8,046,313; 8,069,185; 8,073,804; 8,102,869; 8,103,085; 8,103,600; 8,114,616; 8,155,949; 8,165,916; 8,180,826; 8,250,022; 8,278,057; 8,280,827; 8,285,728; 8,296,247; 8,311,973; 8,315,818; 8,345,940; 8,352,400; 8,364,136; 8,369,967; 8,373,582; 8,392,352; 8,402,490; 8,420,338; 8,457,795; 8,463,553; 8,463,735; 8,484,215; 8,515,884; 8,515,890; 8,516,266; 8,583,263; 8,600,830; 8,605,981; 8,629,789; 8,655,799; 8,682,726; 8,685,741; 8,694,459; 8,709,733; 8,715,943; 8,718,340; 8,768,869; 8,775,441; 8,780,195; 8,873,813; 8,874,477; 8,892,495; 8,949,170; 8,977,614; 8,994,591; 9,009,150; 9,037,589; 9,053,754; 9,063,930; 9,110,991; 9,129,019; 9,151,633; 9,152,877; 9,171,261; 9,173,590; 9,239,951; 9,239,989; 9,262,688; 9,268,330; 9,275,333; 9,311,670; 9,312,973; 9,355,684; 9,361,355; 9,424,533; 9,449,280; 9,465,997; 9,482,672; 9,494,602; 9,503,472; 9,535,563; 9,551,582; 9,563,721; 9,607,103; 9,639,902; 9,646,079; 9,679,251; 9,727,042; 9,732,385; 9,784,748; 9,807,109; 9,818,136; 9,843,488; 9,916,538; 9,918,686; 9,923,860; 9,997,260; RE36926; RE46310; 10,036,757; 10,083,231; 10,086,072; 10,095,741; 10,127,816; 10,147,017; 10,163,137; 10,181,102; 10,242,109; 10,275,906; 10,282,444; 10,324,088; 10,360,905; 10,361,802; 10,380,493; 10,394,871; 10,394,876; 10,409,909; 10,409,910; 10,482,074; 10,482,088; 10,503,755; 10,528,881; 10,542,024; 10,546,001; 20020151992; 20030014191; 20030050923; 20030065661; 20030191682; 20030222819; 20040045030; 20040059754; 20040101914; 2004012384; 20040191804; 20040193789; 20040254902; 20050149459; 20050262179; 20060018548; 20060025158; 20060063156; 200060085736; 20060154276; 20060155398; 26060165012; 20060167784; 2006017874; 26060191534; 20060200253; 20060200258; 20060200259; 20060200260; 26060206344; 20070005545; 20070016476; 26070053513; 20070061022; 20070061023; 20070061735; 20070063875; 26070070038; 20070087756; 20070168056; 26070239314; 20070254295; 20380040749; 20080085524; 20080131439; 28080166719; 20080182280; 20080221910; 28080253645; 20080270332; 20880292194; 20090018984; 20090037235; 28090069088; 20090081711; 20090132441; 28090171871; 23090203588; 20090276391; 20100022408; 20100061185; 20100076642; 20100094560; 20100129838; 20100191369; 20100235285; 20100312798; 20100317420; 20110084513; 20110029922; 20110045476; 20110071969; 20110156896; 20110159521; 20110167110; 20110218956; 20110243417; 20110244465; 20110244887; 20120017232; 20120036016; 20120115170; 20120150651; 20120171672; 20120182294; 20120244558; 20120315630; 20130005596; 20130080369; 20130080370; 20130094750; 20130147598; 20130165070; 20130166387; 20130201316; 20130203053; 20130216118; 20130225439; 20130267431; 20130384675; 20130323255; 20130330008; 20130344621; 20140051594; 20140079297; 20140081793; 20140089241; 20140141983; 20140141990; 20140173452; 20140179549; 20140188094; 20140188462; 20140199709; 20140201126; 20140273006; 20150072879; 20150081444; 20150148997; 20150149155; 20150163242; 20150204559; 20150229661; 20150310301; 20150330985; 20150355195; 20160025500; 20160125302; 20160224951; 20160334401; 20160356790; 20170055130; 20170078400; 20170085588; 20170132931; 20170219805; 20170238879; 20170243126; 20170254806; 20170300804; 20170315117; 20170331899; 20170342493; 20170356032; 20180034912; 20180068358; 20180096307; 20180107734; 20180124181; 20180204111; 20180278693; 20180278694; 20180321254; 20180326581; 20180337836; 20180343304; 20180374210; 20180375940; 20190060449; 20190065961; 20190098090; 20190109904; 20190138907; 20190189236; 20190200888; 20190201691; 20190224441; 20190247662; 20190332623; and 20190378397.

1. A method for assessing hospital acquired infection reduction strategies, comprising:
analyzing, by a computing device, a risk of hospital acquired infections, using supervised learning to generate fuzzy set membership rules;
assessing, by the computing device, resilience based on observed hospital acquired infection risk moderation performance level across a continuum of fuzzy membership sets; and
inferring, by the computing device, a performance of a hospital in hospital acquired infection risk factor prevention employing the fuzzy membership set rules.

2. The method according to claim 1, wherein said analyzing risk comprises selecting risk features according to a principal component analysis.

3. The method according to claim 1, wherein said analyzing risk comprises determining a likelihood of exposure.

4. The method according to claim 1, wherein said analyzing risk comprises determining a likelihood of event reversibility.

5. The method according to claim 1, wherein the hospital acquired infection is methicillin-resistant Staphylococcus aureus.

6. The method according to claim 1, wherein the hospital acquired infection is Clostridioides difficile.

7. The method according to claim 1, wherein the hospital acquired infection risk factor prevention is dependent on at least a cost-effectiveness analysis.

8. The method according to claim 1, wherein the hospital acquired infection risk factor prevention is dependent on at least a patient safety analysis.

9. The method according to claim 1, wherein the risk of hospital acquired infections is analyzed with respect to at least risk event identification, risk mitigation, and risk prevention.

10. The method according to claim 1, wherein the resilience is assessed with respect to an ability of a hospital to anticipate, avoid, and manage hospital acquired infections.

11. The method according to claim 1, wherein said analyzing risk of hospital acquired infections comprises use of fuzzy cognitive mapping to increase reliability and validity of risk mitigation strategies.

12. The method according to claim 1, wherein said analyzing risk of hospital acquired infections comprises use of fuzzy cognitive mapping to assess a stability of a risk event.

13. The method according to claim 1, wherein said analyzing risk of hospital acquired infections comprises use of fuzzy cognitive mapping to assess a reversibility of a risk event.

14. A method for assessing hospital acquired infection risk, comprising:
determining, by a computing system, fuzzy inference system rules based on resilience inference fuzzy membership categories dependent on at least fuzzy risk capacity, resilience capacity, and performance safety;
receiving, by the computing system, information about a hospital acquired infection risk and hospital acquired infection resilience membership function parameters; and
predicting, by the computing system, specific hospital acquired performance safety outcomes by employing a fuzzy inference model dependent on the fuzzy inference system rules and the receiving information.

15. The method according to claim 14, wherein said hospital acquired infection risk is derived though machine learning and fuzzy cognitive mapping.

16. The method according to claim 14, further comprising using fuzzy cognitive mapping to assess a stability of a hospital acquired infection risk event.

17. The method according to claim 14, further comprising using fuzzy cognitive mapping to assess a reversibility of a hospital acquired infection risk event.

18. A system for assessing hospital acquired infection risk, comprising:
at least one server computing device; and
at least one application executable in the at least one server computing device, wherein when executed the at least one application causes the at least one server computing device to at least:
determining, by a computing system, fuzzy inference system rules based on resilience inference fuzzy membership categories dependent on at least fuzzy risk capacity, resilience capacity, and performance safety;
receiving, by the computing system, information about a hospital acquired infection risk and hospital acquired infection resilience membership function parameters; and
predicting, by the computing system, specific hospital acquired performance safety outcomes by employing a fuzzy inference model dependent on the fuzzy inference system rules and the receiving information.

19. The system of claim 18, wherein said hospital acquired infection risk is derived though machine learning and fuzzy cognitive mapping.

20. The system of claim 18, further comprising using fuzzy cognitive mapping to assess a stability of a hospital acquired infection risk event and to assess a reversibility of a hospital acquired infection risk event.
